(19) United States
(12) Patent Application Publication
(10) Pub. No.: US 2025/0073184 A1
(43) Pub. Date: Mar. 6, 2025

(54) CANNABIGEROQUINONE COMPOUNDS, COMPOSITIONS INCLUDING SUCH COMPOUNDS, AND USES OF SUCH COMPOUNDS AND COMPOSITIONS

(71) Applicant: Juva Life, Inc., Redwood City, CA (US)

(72) Inventors: Sanjeev Gangwar, Foster City, CA (US); Guy Miller, Monte Sereno, CA (US)

(21) Appl. No.: 18/694,378

(22) PCT Filed: Oct. 4, 2022
(86) PCT No.: PCT/US2022/045677

§ 371 Date: Mar. 21, 2024

Related U.S. Application Data
(60) Provisional application No. 63/252,921, filed on Oct. 6, 2021.

(51) Int. Cl. A61K 31/122; A61K 31/09; A61K 31/235; C07C 43/315; C07C 49/753; C07C 50/28; C07C 69/24

(52) U.S. Cl. CPC A61K 31/122; A61K 31/09; A61K 31/235; C07C 43/315; C07C 49/753; C07C 50/28; C07C 69/24

(57) ABSTRACT

The present disclosure provides compounds, compositions containing such compounds, and methods of designing, developing, producing and preparing compounds represented by general Formula (I), including pharmaceutically acceptable salts thereof or a synthetic intermediate thereof: The compounds may act as medicaments and may be capable of displaying one or more beneficial therapeutic effects, including treating inflammation and targeting IFNg, IL-6, TNF, IL-1B, Lox-5, IL10, CB2, and/or Lox-15.

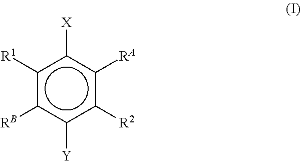

### INCORPORATION BY REFERENCE TO PRIORITY APPLICATION

Any and all applications for which a foreign or domestic priority claim is identified in the Application Data Sheet as filed with the present application are hereby incorporated by reference under 37 C.F.R. § 1.57, and Rules 4.18 and 20.6. This application claims priority to U.S. Provisional Application Ser. No. 63/252,921, filed Oct. 6, 2021, which application is hereby incorporated herein by reference in its entirety.

### BACKGROUND

### Field

The present invention relates to the fields of chemistry and medicine. More particularly, the present invention relates to general small molecule therapeutics, techniques for designing and synthesizing such small molecule therapeutics, compositions comprising small molecule therapeutics, and methods of treating disease comprising administering small molecule therapeutics. In some embodiments, the small molecule therapeutics acts to treat, ameliorate, or prevent the recurrence of an inflammatory condition.

### Description of the Related Technology

Inflammation is part of the biological response of a subject's body to harmful stimuli, such as an injury, illness, allergy, or pathogen, and is a protective response involving immune cells, blood vessels, and inflammatory chemicals. The function of inflammation is to eliminate the initial cause of cell injury, clear out necrotic cells and tissues damaged from the original insult and the inflammatory process, and initiate tissue repair.

Inflammation has long been a well-known symptom of many infectious diseases. However, within the last two decades, molecular and epidemiological research increasingly suggests that it is also intimately linked with a large number of non-infectious diseases, perhaps even all of them. Indeed, inflammation is associated with some of the most significant causes of death in the world today, such as ischemic heart disease, stroke, cancer, diabetes mellitus, chronic kidney disease, non-alcoholic fatty liver disease (NAFLD) and autoimmune and neurodegenerative conditions. The crucial role of inflammatory processes means that the treatment of inflammation may also treat a wide variety of conditions including cancers, autoimmune disorders, and infectious diseases. As such, the development of new compounds for the treatment of inflammation is of interest.

### SUMMARY

Some embodiments provide for a compound having the structure of Formula (I):

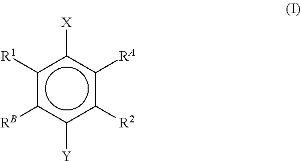

including pharmaceutically acceptable salts thereof. The compound includes:
wherein each of R^{A} and R^{B} is independently selected from the group consisting of: —H, —F, —Cl, —Br, —I, —CH₃, —CH₂CH₃, —CF₃, —OH, —OCH₃, —OCH₂CH₃, —O(CO)NH₂, —O(CO)CH₃, —O(CO)CH₂CH₃, —CN, —NH₂, —NHCH₂CH₂CH₃, —NHCH₂CH₃, —N(CH₃)₂ and —NHCH(CH₃)₂;
wherein each of X and Y is independently selected from the group consisting of: —OH, ═O, —OCH₃, —O(CO)CH₃ and —O(CO)CH₂CH₃;
wherein R¹ is a moiety of formula (II):

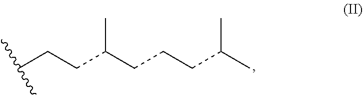

wherein each dashed bond is individually selected from representing a carbon-carbon single bond and representing a carbon-carbon double bond;
wherein R² is either a moiety of the formula (III-A), —(CH₂)ₙCH₃, wherein n in an integer greater than one and less than seven, or a moiety of the formula (III-B), —(CH₂)ₘ(CH═CH)(CH₂)ₚCH₃, wherein m is an integer greater than zero and less than four, and wherein p is an integer greater than or equal to zero and less than four, and wherein the sum of m and p is not greater than four; and
with the caveat that the compound is not any of the following compounds (IV-VIII):

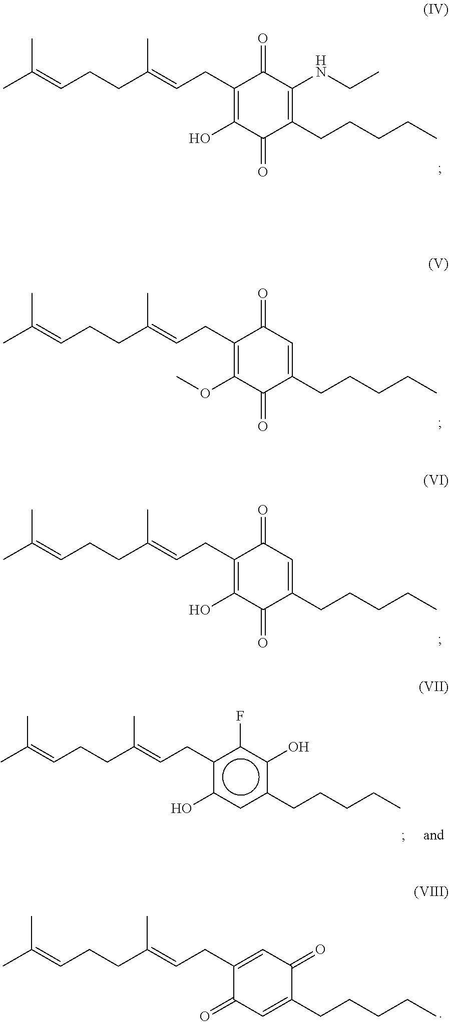

In certain embodiments of Formula (I), R^{B} is —OH. In some embodiments, R^{B} is selected from the group consisting of —OCH₃, —OCH₂CH₃, —O(CO)CH₃, —O(CO)CH₂CH₃, and —NH₂. In some embodiments, R^{A} is —H. In some embodiments, R^{A} is selected from the group consisting of —F, —Cl, and —Br. In some embodiments, each of X and Y is ═O. In some embodiments, each of X and Y is independently selected from the group consisting of —OCOCH₃ and —O(CO)CH₂CH₃. In some embodiments, Y is ═O. In some embodiments, R² is a five-carbon moiety. In some embodiments, Y is selected from the group consisting of —OCOCH₃ and —O(CO)CH₂CH₃. In some embodiments, the compound inhibits an activity selected from the group consisting of TNF, IL-6, IL-1β, IFNg, Lox5, Lox15, IL-10, CB2, and combinations thereof with an EC50 that is at most about 20 μM. In some embodiments, the compound inhibits TNF activity with an EC50 that is at most about 20 μM. In some embodiments, the compound inhibits IL-6 activity with an EC50 that is at most about 20 μM. In some embodiments, the compound inhibits IL-10 activity with an EC50 that is at most about 10 PM. In some embodiments, the compound inhibits IFNg activity with an EC50 that is at most about 1 μM. In some embodiments, the compound inhibits Lox5 activity with an EC50 that is at most about 0.3 μM. In some embodiments, the compound inhibits Lox15 activity with an EC50 that is at most about 1 μM. In some embodiments, the compound has at least about 100-fold decreased toxicity to cells relative to staurosporine. In some embodiments, the compound has at least about 2 fold selectivity for inhibiting the CB2 receptor over the CB1 receptor.

Some embodiments provide a compound of Formula (I) as part of a composition along with at least one of a pharmaceutically effective carrier, a pharmaceutically effective diluent and a pharmaceutically effective excipient. In some embodiments, compound is formulated for intradermal, topical, transdermal, oral, buccal, sublingual, nasal or intravenous application.

Certain embodiments provide for a compound having the structure of Formula (I):

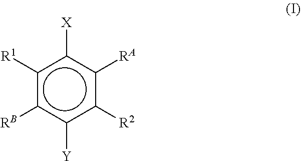

including pharmaceutically acceptable salts thereof. The compound includes:
wherein R^{A} is selected from the group consisting of: —H, —F, —Cl, —Br, —I, —CH₃, —CH₂CH₃, —CF₃, —OH, —OCH₃, —OCH₂CH₃, —O(CO)NH₂, —CN, —NH₂, —NHCH₂CH₂CH₃, —NHCH₂CH₃, —N(CH₃)₂ and —NHCH(CH₃)₂; wherein R^{B} is selected from the group consisting of —H, —Cl, —Br, —I, —CH₃, —CH₂CH₃, —CF₃, —OH, —OCH₂CH₃, —OCONH₂, —O(CO)CH₃, —O(CO)CH₂CH₃—CN, —NH₂, —NHCH₂CH₂CH₃, —NHCH₂CH₃, —N(CH₃)₂ and —NHCH(CH₃)₂; wherein each of X and Y is independently selected from the group consisting of: —OH, ═O, —OCH₃, —OCOCH₃ and —O(CO)CH₂CH₃; wherein R¹ is a moiety of formula (II):

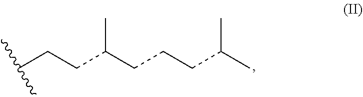

wherein each dashed line is independently selected from representing a carbon-carbon single bond and representing a carbon-carbon double bond; and wherein R² is either a moiety of the formula (III-A), —(CH₂)ₙCH₃, wherein n in an integer greater than one and less than seven, or a moiety of the formula (III-B), —(CH₂)ₘ(CH═CH)(CH₂)ₚCH₃, wherein m is an integer greater than zero and less than four, and wherein p is an integer greater than or equal to zero and less than four, and wherein the sum of m and p is not greater than four; and wherein if R^{B} is —OH, then R^{A} is neither —H nor —NHCH₂CH₃; and wherein R^{A} and R^{B} are not both —H.

In certain embodiments, R^{B} is —OH. In some embodiments, R^{B} is selected from the group consisting of —OCH₃, —OCH₂CH₃, —O(CO)CH₃, —O(CO)CH₂CH₃, and —NH₂. In some embodiments, R^{A} is —H. In some preferred embodiments, R^{A} is selected from the group consisting of —F, —Cl, and —Br. In some embodiments, each of X and Y is ═O. In some embodiments, each of X and Y is independently selected from the group consisting of —OCOCH₃ and —O(CO)CH₂CH₃. In some embodiments, R² is a five-carbon moiety. In some embodiments, Y is ═O. In some embodiments, Y is selected from the group consisting of —OCOCH₃ and —O(CO)CH₂CH₃. In some embodiments, the compound inhibits an activity selected from the group consisting of TNF, IL-6, IL-1β, IFNg, Lox5, Lox15, IL-10, CB2, and combinations thereof with an EC50 that is at most about 20 μM. In some embodiments, the compound inhibits TNF activity with an EC50 that is at most about 20 μM. In some embodiments, the compound inhibits IL-6 activity with an EC50 that is at most about 20 μM. In some embodiments, the compound inhibits IL-1β activity with an EC50 that is at most about 10 μM. In some embodiments, the compound inhibits IFNg activity with an EC50 that is at most about 1 μM. In some embodiments, the compound inhibits Lox5 activity with an EC50 that is at most about 0.3 μM. In some embodiments, the compound inhibits Lox15 activity with an EC50 that is at most about 1 μM. In some embodiments, the compound has at least about 100-fold decreased toxicity to cells relative to staurosporine. In some embodiments, the compound has at least about 2 fold selectivity for inhibiting the CB2 receptor over the CB1 receptor.

Some embodiments provide a compound of Formula (I) as part of a composition along with at least one of a pharmaceutically effective carrier, a pharmaceutically effective diluent and a pharmaceutically effective excipient. In some embodiments, compound is formulated for intradermal, topical, transdermal, oral, buccal, sublingual, nasal or intravenous application.

Also disclosed herein are methods of treating, ameliorating, or preventing the recurrence of an inflammatory condition selected from the group consisting of acute inflammation, chronic inflammation, developmental inflammation, meta-inflammation and inflammaging in a subject having an inflammatory condition. In some embodiments, the method comprises administering an effective dose of a composition comprising (i) at least one of a pharmaceutically effective carrier, a pharmaceutically effective diluent, and a pharmaceutically effective excipient, and (ii) a compound of Formula (I):

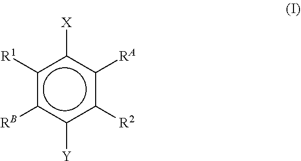

wherein R^{A} and R^{B} are each selected from the group consisting of: —H, —F, —Cl, —Br, —I, —CH₃, —CH₂CH₃, —CF₃, —OH, —OCH₃, —OCH₂CH₃, —OCONH₂, —O(CO)CH₃, —O(CO)CH₂CH₃, —CN, —NH₂, —NHCH₂CH₂CH₃, —NHCH₂CH₃, —N(CH₃)₂ and —NHCH(CH₃)₂; wherein each of X and Y are independently selected from the group consisting of: —OH, ═O, —OCH₃, —OCOCH₃ and —OCOCH₂CH₃; wherein R¹ is a moiety of formula (II):

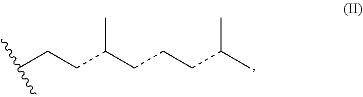

wherein each dashed line is independently selected from representing a carbon-carbon single bond and representing a carbon-carbon double bond; wherein R² is either a moiety of the formula (III-A), —(CH₂)ₙCH₃, wherein n in an integer greater than one and less than seven, or a moiety of the formula (III-B), —(CH₂)ₘ(CH═CH)(CH₂)ₚCH₃, wherein m is an integer greater than zero and less than four, and wherein p is an integer greater than or equal to zero and less than four, and wherein the sum of m and p is not greater than four; and with the caveat that the compound is not any of the following compounds (IV, VI and VIII):

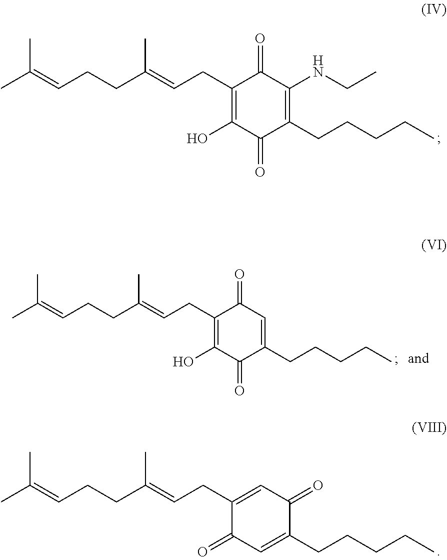

In certain embodiments, the chronic inflammation results from at least one of anemia, arthritis, asthma, autoimmune disease, bone disease, bowel disease, cancer, cardiovascular disease, celiac disease, cerebrovascular disease, Crone's disease, diabetes, dysglycemia, eczema, fibromyalgia, gastrointestinal disorder, gingivitis, granulomatosis, Grave's disease, Hashimoto's disease, hemolytic anemia, inflammatory bowel disease, joint disease, leukemia, lupus, metabolic disease, muscular dystrophy, neuropathy, obesity, ocular disease, periodontitis, psoriasis, pulmonary disease, renal disease, rheumatoid arthritis, scleroderma, sclerosis, skin disease, thyroid disease, thyroiditis, ulceratic colitis, vitiligo, Wegener's disease, Castleman's disease, pulmonary arterial hypentension, atopic dermititus, and sciatica. In some embodiments, the chronic inflammation is associated with at least one of anemia, arthritis, asthma, autoimmune disease, bone disease, bowel disease, cancer, cardiovascular disease, celiac disease, cerebrovascular disease, Crone's disease, diabetes, dysglycemia, eczema, fibromyalgia, gastrointestinal disorder, gingivitis, granulomatosis, Grave's disease, Hashimoto's disease, hemolytic anemia, inflammatory bowel disease, joint disease, leukemia, lupus, metabolic disease, muscular dystrophy, neuropathy, obesity, ocular disease, periodontitis, psoriasis, pulmonary disease, renal disease, rheumatoid arthritis, scleroderma, sclerosis, skin disease, thyroid disease, thyroiditis, ulceratic colitis, vitiligo, Wegener's disease, Castleman's disease, pulmonary arterial hypentension, atopic dermititus, and sciatica. In some embodiments, the inflammation is not neuroinflammation. In some embodiments, the therapeutic effect of the compound is not attributed to its interaction with a one or more PPAR-γ receptor in the subject. In some embodiments, the compound inhibits an activity selected from the group consisting of TNF, IL-6, IL-1β, IFNg, Lox5, Lox15, IL-10, CB2, and combinations thereof with an EC50 that is at most about 20 μM. In some embodiments, the compound has at least about 100-fold decreased toxicity to cells relative to staurosporine. In some embodiments, the compound has at least about 2 fold selectivity for inhibiting the CB2 receptor over the CB1 receptor. In some embodiments, the compound inhibits TNF activity with an EC50 that is at most about 20 μM. In some embodiments, the compound inhibits IL-6 activity with an EC50 that is at most about 20 μM. In some embodiments, the compound inhibits IL-10 activity with an EC50 that is at most about 10 μM. In some embodiments, the compound inhibits IFNg activity with an EC50 that is at most about 1 μM. In some embodiments, the compound inhibits Lox5 activity with an EC50 that is at most about 0.3 μM. In some embodiments, the compound inhibits Lox15 activity with an EC50 that is at most about 1 μM.

Also disclosed herein is a use of a composition in the preparation of a medicament for treating, ameliorating, or preventing the recurrence of an inflammatory condition selected from the group consisting of acute inflammation, chronic inflammation, developmental inflammation, meta-inflammation and inflammaging in a subject having an inflammatory condition, wherein the composition comprises (i) at least one of a pharmaceutically effective carrier, a pharmaceutically effective diluent, and a pharmaceutically effective excipient, and (ii) a compound of formula (I):

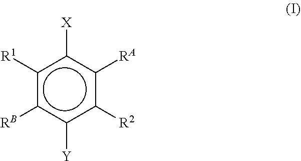

wherein each of R^{A} and R^{B} is independently selected from the group consisting of: —H, —F, —Cl, —Br, —I, —CH₃, —CH₂CH₃, —CF₃, —OH, —OCH₃, —OCH₂CH₃, —O(CO)NH₂, —O(CO)CH₃, —O(CO)CH₂CH₃, —CN, —NH₂, —NHCH₂CH₂CH₃, —NHCH₂CH₃, —N(CH₃)₂ and —NHCH(CH₃)₂;
wherein each of X and Y is independently selected from the group consisting of: —OH, ═O, —OCH₃, —O(CO)CH₃ and —O(CO)CH₂CH₃; wherein R¹ is a moiety of formula (II):

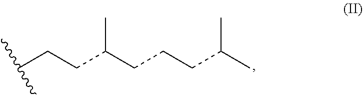

wherein each dashed bond is individually selected from representing a carbon-carbon single bond and representing a carbon-carbon double bond; wherein R² is a moiety of formula (III):

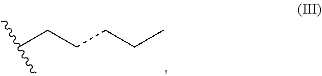

wherein the dashed bond is selected from representing a carbon-carbon single bond and representing a carbon-carbon double bond; and wherein the disease or disorder is associated with the activity of or the expression of one or more of TNF, IL-6, IL-1β, IL-10, IFNg, Lox5, CB2 and/or Lox15. In some embodiments, the compound inhibits an activity selected from the group consisting of TNF, IL-6, IL-1β, IFNg, Lox5, Lox15, IL-10, CB2, and combinations thereof with an EC50 that is at most about 20 μM. In some embodiments, the compound has at least about 100-fold decreased toxicity to cells relative to staurosporine. In some embodiments, the compound has at least about 2 fold selectivity for inhibiting the CB2 receptor over the CB1 receptor.

In some embodiments, R^{B} is —OH. In some embodiments, R^{B} is selected from the group consisting of —OCH₃, —OCH₂CH₃, —O(CO)CH₃, —O(CO)CH₂CH₃, and —NH₂. In some embodiments, R^{A} is —H. In some embodiments, R^{A} is selected from the group consisting of —F, —Cl, and —Br. In some embodiments, each of X and Y is ═O. In some embodiments, each of X and Y is independently selected from the group consisting of —OCOCH₃ and —O(CO)CH₂CH₃. In some embodiments, R² is a five-carbon moiety. In some embodiments, Y is ═O. In some embodiments, Y is selected from the group consisting of —OCOCH₃ and —O(CO)CH₂CH₃. In some embodiments, the compound is selected from compounds IV-VIII. In some embodiments, the compound is compound VI. In some embodiments, the subject is mammalian or human. In some embodiments, the one or more symptom is selected from the group consisting of systemic inflammation, acute inflammation, chronic inflammation, developmental inflammation, meta-inflammation, and inflammaging. In some embodiments, the method of treatment results in the amelioration, reduction, or prevention of inflammation in the subject. In some embodiments, the compound is formulated with at least one of a pharmaceutically effective carrier, a pharmaceutically effective diluent, and a pharmaceutically effective excipient. In some embodiments, the compound is formulated for intradermal, topical, transdermal, oral, buccal, sublingual, nasal, or intravenous application. In some embodiments, the compound inhibits an activity selected from the group consisting of TNF, IL-6, IL-1β, IFNg, Lox5, Lox15, IL-10, CB2, and combinations thereof with an EC50 that is at most about 20 μM. In some embodiments, the compound has at least about 100-fold decreased toxicity to cells relative to staurosporine. In some embodiments, the compound has at least about 2 fold selectivity for inhibiting the CB2 receptor over the CB1 receptor. In some embodiments, the disease or disorder is associated with the activity and/or expression of TNF. In some embodiments, the compound inhibits TNF activity with an EC50 that is at most about 20 μM. In some embodiments, the disease or disorder is selected from the group consisting of acute myeloid leukemia, acute respiratory distress, amyloidosis, ankylosing spondylitis, arthritis, autoimmune disease, axial spondyloarthritis, back pain, Behcet's syndrome, burn-associated inflammation, cancer, cardiovascular disease, cerebral malaria, chronic lymphocytic leukemia, Crohn's disease, diabetes, Dupuytren's disease, fibrosis, fingernail psoriasis, fracture, Grave's disease, hidradenitis suppurativa, HIV infection, immune-mediated inflammatory disease, infectious disease, inflammatory bowel disease, influenza, injury, joint pain, lupus, multiple sclerosis, neck pain, non-Infectious Intermediate, Posterior, and Panuveitis, obesity, onchocerciasis, organ injury, osteoarthritis, pediatric Crohn's disease, pediatric plaque psoriasis, pediatric ulcerative colitis, plaque psoriasis, polyarticular juvenile idiopathic arthritis, post-operative cognitive dysfunction, psoriatic arthritis, rheumatoid arthritis, sepsis, spondyloarthritis, systemic lupus erythematosus nephritis, tissue damage, trypanosomiasis, type I diabetes, ulcerative colitis, uveitis, and ventilator-induced lung injury. In some embodiments, the disease or disorder is associated with the activity and/or expression of IL-6. In some embodiments, the compound inhibits IL-6 activity with an EC50 that is at most about 20 μM. In some embodiments, the disease or disorder is selected from the group consisting of acquired hemophilia A, adult onset Still's disease, amyloid A amyloidosis, ankylosing spondylitis, autoimmune disease, autoimmune hemolytic anemia, atopic dermatitis, Behcet's disease, burn-associated inflammation, cancer, cardiovascular disease, cardiovascular disease in rheumatoid arthritis, Castleman's disease, chronic glomerulonephritis, colorectal cancer, Crohn's disease, cryoglobulinemia, diabetes, giant cell arteritis, acute graft-versus-host disease, graft-versus-host disease, Grave's disease, Grave's ophthalmopathy, hepatitis B infection, HIV infection, HTLV-1 infection, infectious inflammation, injury-associated inflammation, KSHV infection, lupus, lupus erythematosus, myeloperoxidase-antineutrophil cytoplasmic antibody-associated crescentic glomerulonephritis, neuromyelitis optica, non-infectious inflammation, obesity, non-ST-elevation myocardial infarction, organ rejection, organ transplant rejection, polychondritis, polymyalgia rheumatica, polymyositis, pulmonary arterial hypertension, relapsing polychondritis, remitting seronegative symmetrical synovitis with pitting edema, rheumatoid arthritis, rheumatoid vasculitis, sciatica, sclerosis, spondyloarthritis, systemic juvenile idiopathic arthritis, systemic lupus erythematosus, systemic sclerosis, Takayasu arteritis, tumor necrosis factor receptor-associated periodic syndrome, type II diabetes, uveitis, and vasculitis syndrome. In some embodiments, the disease or disorder is associated with the activity and/or expression of IL-1β. In some embodiments, the compound inhibits IL-1β activity with an EC50 that is at most about 10 μM. In some embodiments, the disease or disorder is selected from the group consisting of acne, acute respiratory distress syndrome, amyloidosis, antisynthetase syndrome, arthritis, atherosclerosis, autoinflammation, autoimmune disease, Behcet's disease, Blau syndrome, bone disease, cancer, cardiovascular disease, chondrocalcinosis, chronic inflammatory neurological cutaneous articular syndrome, chronic obstructive pulmonary disease, Crohn's disease, cryopyrin-associated periodic syndrome, deficiency in IL-1 receptor antagonist, diabetes, disseminated intravascular coagulation, Erdheim-Chester syndrome, familial cold-induced autoinflammatory syndrome, familial Mediterranean fever, gout, graft-versus-host disease, headache, heart failure, hyper IgD syndrome, hyperostosis, hypoglycemia, inanition, infectious disease, inflammasome-associated disease, injury, interstitial lung disease, intestine inflammation, inflammasome-associated disease, irritable bowel syndrome, ischemic disease, joint disease, macrophage activation syndrome, macular degeneration, Majeed syndrome, malignancy, metabolic syndrome disorder, mevalonate kinase deficiency syndrome, microbial infection, Muckle-Wells syndrome, multiple sclerosis, myeloma, myocardial infarction, non-cancer inflammatory disease, obesity, ocular disease, osteitis, osteoarthritis, pericarditis, PFAPA syndrome, post myocardial infarction heart failure, psoriasis, pulmonary disease, pustulosis, pyoderma gangrenosum, pyogenic arthritis, pyogenic arthritis-pyoderma gangrenosum-acne syndrome, recurrent idiopathic pericarditis, recurrent pericarditis, redness at injection site, relapsing chondritis, renal dysfunction, retinal degeneration, rheumatoid arthritis, Schnitzler syndrome, sepsis, septic shock syndrome, sclerosis, sinus inflammation, Sjögren syndrome, smoldering multiple myeloma, Still's disease, Sweet syndrome, synovitis, systemic lupus erythematosus, systemic-onset juvenile idiopathic arthritis, TNF receptor-associated periodic syndrome, type I diabetes, type II diabetes, upper respiratory tract inflammation, urticarial vasculitis, and uveitis. In some embodiments, the disease or disorder is associated with the activity and/or expression of IFNg. In some embodiments, the compound inhibits IFNg activity with an EC50 that is at most about 1 μM. In some embodiments, the disease or disorder is selected from the group consisting of anti-TNF-induced lupus, arthritis, autoimmune diabetes, autoimmune disease, autoimmune encephalomyelitis, autoimmune myositis, Behcet's disease, cutaneous autoimmune disorder, dermatomyositis, diabetes, infectious disease, inflammatory bowel disease, injury, juvenile idiopathic arthritis, leukocytoclastic vasculitis, lupus, metabolic immune disorder, multiple sclerosis, obesity, psoriatic arthritis, psoriasis, psoriasiform eruptions, rheumatoid arthritis, sclerosis, Sjogren's syndrome, synovial inflammation, systemic lupus erythematosus, systemic sclerosis, thrombosis, type I diabetes, and vasculitis. In some embodiments, the disease or disorder is associated with the activity and/or expression of Lox5. In some embodiments, the compound inhibits Lox5 activity with an EC50 that is at most about 0.3 μM. In some embodiments, the disease or disorder is selected from the group consisting of an allergic reaction, allergy-associated inflammation, angioedema, arthritis, asthma, atherosclerosis, autoimmune disorder, burn-associated inflammation, cardiovascular disease, cancer, chronic obstructive pulmonary disease, conjunctivitis, diabetes, eczema, H. pylori infection, infectious disease, inflammatory bowel disease, injury, NSAID hypersensitivity reaction, NSAID-induced non-allergic reaction, obesity, psoriasis, rheumatoid arthritis, rhinitis, and urticaria. In some embodiments, the disease or disorder is associated with the activity and/or expression of Lox15. In some embodiments, the compound inhibits Lox15 activity with an EC50 that is at most about 1 μM. In some embodiments, the disease or disorder is selected from the group consisting of an allergic reaction, allergy-associated inflammation, angioedema, arthritis, asthma, atherosclerosis, autoimmune disorder, burn-associated inflammation, cardiovascular disease, cancer, chronic obstructive pulmonary disease, conjunctivitis, diabetes, eczema, H. pylori infection, infectious disease, inflammatory bowel disease, injury, NSAID hypersensitivity reaction, NSAID-induced non-allergic reaction, obesity, psoriasis, rheumatoid arthritis, rhinitis, and urticaria.

Also disclosed herein is a method of treating, ameliorating, reducing, or preventing the recurrence of a one or more symptom of a disease or disorder in a subject in need thereof. In some embodiments, the method comprises administering a compound of formula (I):

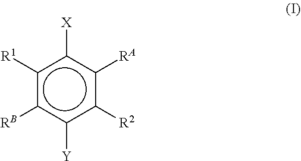

wherein each of R^{A} and R^{B} is independently selected from the group consisting of: —H, —F, —Cl, —Br, —I, —CH₃, —CH₂CH₃, —CF₃, —OH, —OCH₃, —OCH₂CH₃, —O(CO)NH₂, —O(CO)CH₃, —O(CO)CH₂CH₃, —CN, —NH₂, —NHCH₂CH₂CH₃, —NHCH₂CH₃, —N(CH₃)₂ and —NHCH(CH₃)₂;
wherein each of X and Y is independently selected from the group consisting of: —OH, ═O, —OCH₃, —O(CO)CH₃ and —O(CO)CH₂CH₃; wherein R¹ is a moiety of formula (II):

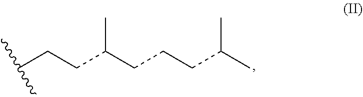

wherein each dashed bond is individually selected from representing a carbon-carbon single bond and representing a carbon-carbon double bond; wherein R² is a moiety of formula (III):

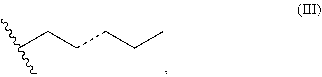

wherein the dashed bond is selected from representing a carbon-carbon single bond and representing a carbon-carbon double bond; and wherein the disease or disorder is associated with the activity of or the expression of one or more of TNF, IL-6, IL-1β, IL-10, IFNg, Lox5, CB2, and/or Lox15.

In some embodiments, R^{B} is —OH. In some embodiments, R^{B} is selected from the group consisting of —OCH₃, —OCH₂CH₃, —O(CO)CH₃, —O(CO)CH₂CH, and —NH₂. In some embodiments, R^{A} is —H. In some embodiments, R^{A} is selected from the group consisting of —F, —Cl, and —Br. In some embodiments, each of X and Y is ═O. In some embodiments, each of X and Y is independently selected from the group consisting of —OCOCH₃ and —O(CO)CH₂CH₃. In some embodiments, R² is a five-carbon moiety. In some embodiments, Y is ═O. In some embodiments, Y is selected from the group consisting of —OCOCH₃ and —O(CO)CH₂CH₃. In some embodiments, the compound is selected from compounds IV-VIII. In some embodiments, the compound is compound VI. In some embodiments, the subject is mammalian or human. In some embodiments, the one or more symptom is selected from the group consisting of systemic inflammation, acute inflammation, chronic inflammation, developmental inflammation, meta-inflammation, and inflammaging. In some embodiments, the method of treatment results in the amelioration, reduction, or prevention of inflammation in the subject. In some embodiments, the compound is formulated with at least one of a pharmaceutically effective carrier, a pharmaceutically effective diluent, and a pharmaceutically effective excipient. In some embodiments, the compound is formulated for intradermal, topical, transdermal, oral, buccal, sublingual, nasal, or intravenous application. In some embodiments, the compound inhibits an activity selected from the group consisting of TNF, IL-6, IL-1β, IFNg, Lox5, Lox15, IL-10, CB2, and combinations thereof with an EC50 that is at most about 20 μM. In some embodiments, the compound has at least about 100-fold decreased toxicity to cells relative to staurosporine. In some embodiments, the compound has at least about 2 fold selectivity for inhibiting the CB2 receptor over the CB1 receptor. In some embodiments, the disease or disorder is associated with the activity and/or expression of TNF. In some embodiments, the compound inhibits TNF activity with an EC50 that is at most about 20 μM. In some embodiments, the disease or disorder is selected from the group consisting of acute myeloid leukemia, acute respiratory distress, amyloidosis, ankylosing spondylitis, arthritis, autoimmune disease, axial spondyloarthritis, back pain, Behcet's syndrome, burn-associated inflammation, cancer, cardiovascular disease, cerebral malaria, chronic lymphocytic leukemia, Crohn's disease, diabetes, Dupuytren's disease, fibrosis, fingernail psoriasis, fracture, Grave's disease, hidradenitis suppurativa, HIV infection, immune-mediated inflammatory disease, infectious disease, inflammatory bowel disease, influenza, injury, joint pain, lupus, multiple sclerosis, neck pain, non-Infectious Intermediate, Posterior, and Panuveitis, obesity, onchocerciasis, organ injury, osteoarthritis, pediatric Crohn's disease, pediatric plaque psoriasis, pediatric ulcerative colitis, plaque psoriasis, polyarticular juvenile idiopathic arthritis, post-operative cognitive dysfunction, psoriatic arthritis, rheumatoid arthritis, sepsis, spondyloarthritis, systemic lupus erythematosus nephritis, tissue damage, trypanosomiasis, type I diabetes, ulcerative colitis, uveitis, and ventilator-induced lung injury. In some embodiments, the disease or disorder is associated with the activity and/or expression of IL-6. In some embodiments, the compound inhibits IL-6 activity with an EC50 that is at most about 20 μM. In some embodiments, the disease or disorder is selected from the group consisting of acquired hemophilia A, adult onset Still's disease, amyloid A amyloidosis, ankylosing spondylitis, autoimmune disease, autoimmune hemolytic anemia, atopic dermatitis, Behcet's disease, burn-associated inflammation, cancer, cardiovascular disease, cardiovascular disease in rheumatoid arthritis, Castleman's disease, chronic glomerulonephritis, colorectal cancer, Crohn's disease, cryoglobulinemia, diabetes, giant cell arteritis, acute graft-versus-host disease, graft-versus-host disease, Grave's disease, Grave's ophthalmopathy, hepatitis B infection, HIV infection, HTLV-1 infection, infectious inflammation, injury-associated inflammation, KSHV infection, lupus, lupus erythematosus, myeloperoxidase-antineutrophil cytoplasmic antibody-associated crescentic glomerulonephritis, neuromyelitis optica, non-infectious inflammation, obesity, non-ST-elevation myocardial infarction, organ rejection, organ transplant rejection, polychondritis, polymyalgia rheumatica, polymyositis, pulmonary arterial hypertension, relapsing polychondritis, remitting seronegative symmetrical synovitis with pitting edema, rheumatoid arthritis, rheumatoid vasculitis, sciatica, sclerosis, spondyloarthritis, systemic juvenile idiopathic arthritis, systemic lupus erythematosus, systemic sclerosis, Takayasu arteritis, tumor necrosis factor receptor-associated periodic syndrome, type II diabetes, uveitis, and vasculitis syndrome. In some embodiments, the disease or disorder is associated with the activity and/or expression of IL-1β. In some embodiments, the compound inhibits IL-1β activity with an EC50 that is at most about 10 μM. In some embodiments, the disease or disorder is selected from the group consisting of acne, acute respiratory distress syndrome, amyloidosis, antisynthetase syndrome, arthritis, atherosclerosis, autoinflammation, autoimmune disease, Behcet's disease, Blau syndrome, bone disease, cancer, cardiovascular disease, chondrocalcinosis, chronic inflammatory neurological cutaneous articular syndrome, chronic obstructive pulmonary disease, Crohn's disease, cryopyrin-associated periodic syndrome, deficiency in IL-1 receptor antagonist, diabetes, disseminated intravascular coagulation, Erdheim-Chester syndrome, familial cold-induced autoinflammatory syndrome, familial Mediterranean fever, gout, graft-versus-host disease, headache, heart failure, hyper IgD syndrome, hyperostosis, hypoglycemia, inanition, infectious disease, inflammasome-associated disease, injury, interstitial lung disease, intestine inflammation, inflammasome-associated disease, irritable bowel syndrome, ischemic disease, joint disease, macrophage activation syndrome, macular degeneration, Majeed syndrome, malignancy, metabolic syndrome disorder, mevalonate kinase deficiency syndrome, microbial infection, Muckle-Wells syndrome, multiple sclerosis, myeloma, myocardial infarction, non-cancer inflammatory disease, obesity, ocular disease, osteitis, osteoarthritis, pericarditis, PFAPA syndrome, post myocardial infarction heart failure, psoriasis, pulmonary disease, pustulosis, pyoderma gangrenosum, pyogenic arthritis, pyogenic arthritis-pyoderma gangrenosum-acne syndrome, recurrent idiopathic pericarditis, recurrent pericarditis, redness at injection site, relapsing chondritis, renal dysfunction, retinal degeneration, rheumatoid arthritis, Schnitzler syndrome, sepsis, septic shock syndrome, sclerosis, sinus inflammation, Sjögren syndrome, smoldering multiple myeloma, Still's disease, Sweet syndrome, synovitis, systemic lupus erythematosus, systemic-onset juvenile idiopathic arthritis, TNF receptor-associated periodic syndrome, type I diabetes, type II diabetes, upper respiratory tract inflammation, urticarial vasculitis, and uveitis. In some embodiments, the disease or disorder is associated with the activity and/or expression of IFNg. In some embodiments, the compound inhibits IFNg activity with an EC50 that is at most about 1 μM. In some embodiments, the disease or disorder is selected from the group consisting of anti-TNF-induced lupus, arthritis, autoimmune diabetes, autoimmune disease, autoimmune encephalomyelitis, autoimmune myositis, Behcet's disease, cutaneous autoimmune disorder, dermatomyositis, diabetes, infectious disease, inflammatory bowel disease, injury, juvenile idiopathic arthritis, leukocytoclastic vasculitis, lupus, metabolic immune disorder, multiple sclerosis, obesity, psoriatic arthritis, psoriasis, psoriasiform eruptions, rheumatoid arthritis, sclerosis, Sjogren's syndrome, synovial inflammation, systemic lupus erythematosus, systemic sclerosis, thrombosis, type I diabetes, and vasculitis. In some embodiments, the disease or disorder is associated with the activity and/or expression of Lox5. In some embodiments, the compound inhibits Lox5 activity with an EC50 that is at most about 0.3 μM. In some embodiments, the disease or disorder is selected from the group consisting of an allergic reaction, allergy-associated inflammation, angioedema, arthritis, asthma, atherosclerosis, autoimmune disorder, burn-associated inflammation, cardiovascular disease, cancer, chronic obstructive pulmonary disease, conjunctivitis, diabetes, eczema, H. pylori infection, infectious disease, inflammatory bowel disease, injury, NSAID hypersensitivity reaction, NSAID-induced non-allergic reaction, obesity, psoriasis, rheumatoid arthritis, rhinitis, and urticaria. In some embodiments, the disease or disorder is associated with the activity and/or expression of Lox15. In some embodiments, the compound inhibits Lox15 activity with an EC50 that is at most about 1 μM. In some embodiments, the disease or disorder is selected from the group consisting of an allergic reaction, allergy-associated inflammation, angioedema, arthritis, asthma, atherosclerosis, autoimmune disorder, burn-associated inflammation, cardiovascular disease, cancer, chronic obstructive pulmonary disease, conjunctivitis, diabetes, eczema, H. pylori infection, infectious disease, inflammatory bowel disease, injury, NSAID hypersensitivity reaction, NSAID-induced non-allergic reaction, obesity, psoriasis, rheumatoid arthritis, rhinitis, and urticaria.

Also disclosed herein is a use of a compound of formula (I) in the preparation of a medicament for treating, ameliorating, reducing, or preventing the recurrence of a one or more symptom of a disease or disorder in a subject in need thereof, wherein the compound of formula (I) is:

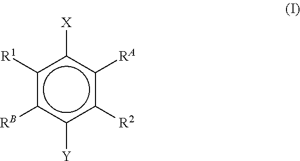

wherein each of R^{A} and R^{B} is independently selected from the group consisting of: —H, —F, —Cl, —Br, —I, —CH₃, —CH₂CH₃, —CF₃, —OH, —OCH₃, —OCH₂CH₃, —O(CO)NH₂, —O(CO)CH₃, —O(CO)CH₂CH₃, —CN, —NH₂, —NHCH₂CH₂CH₃, —NHCH₂CH₃, —N(CH₃)₂ and —NHCH(CH₃)₂;
wherein each of X and Y is independently selected from the group consisting of: —OH, ═O, —OCH₃, —O(CO)CH₃ and —O(CO)CH₂CH₃;
wherein R¹ is a moiety of formula (II):

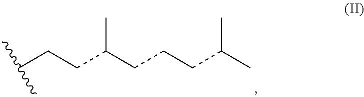

wherein each dashed bond is individually selected from representing a carbon-carbon single bond and representing a carbon-carbon double bond;
wherein R² is a moiety of formula (III):

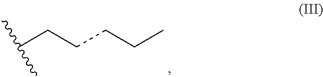

wherein the dashed bond is selected from representing a carbon-carbon single bond and representing a carbon-carbon double bond; and
wherein the disease or disorder is associated with the activity of or the expression of one or more of TNF, IL-6, IL-1β, IL10, IFNg, Lox5, CB2, and/or Lox15.

Also disclosed herein is a compound selected from the group comprising:

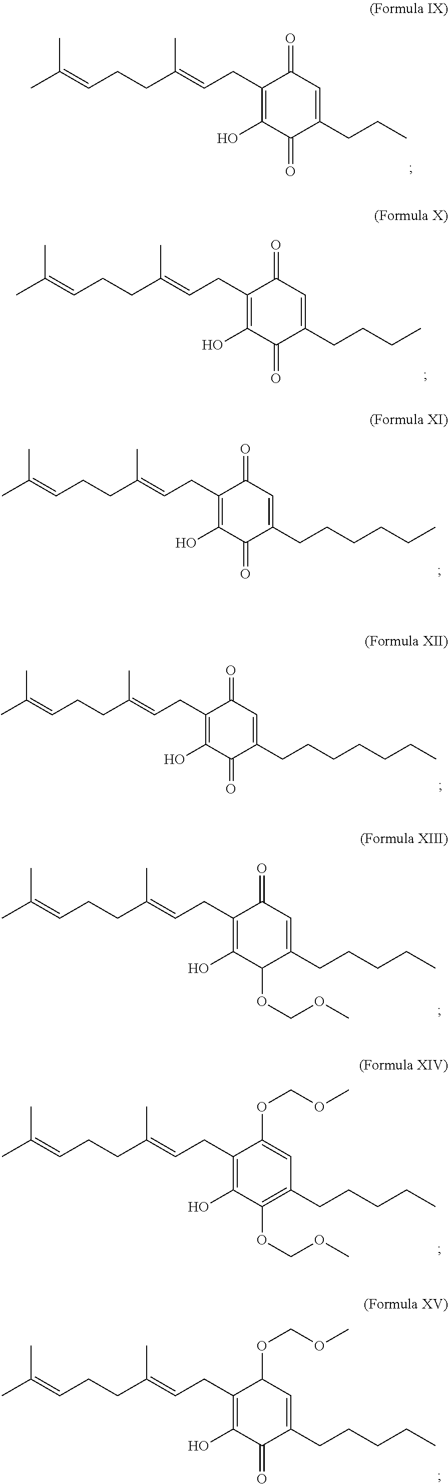

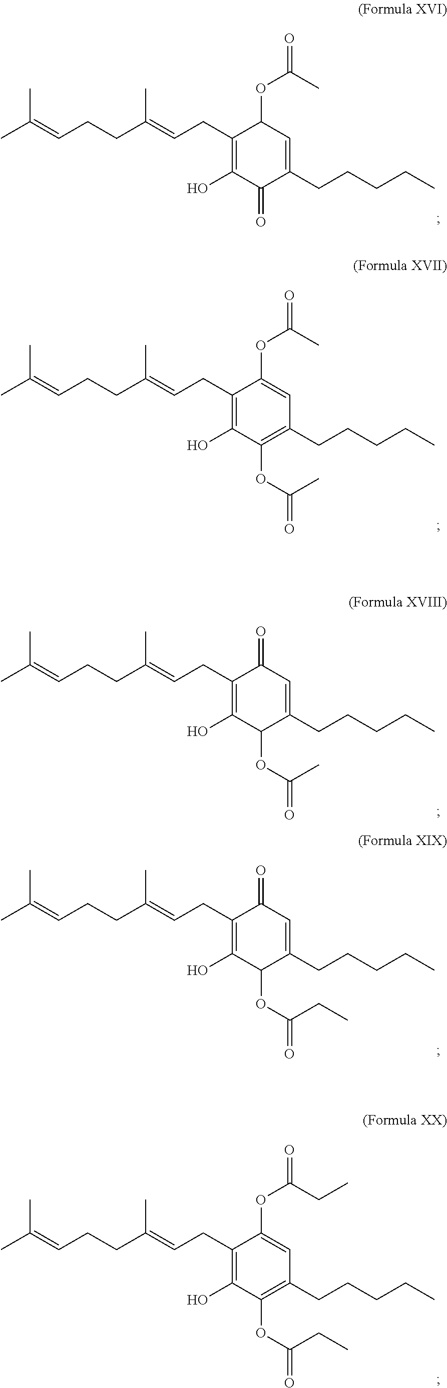

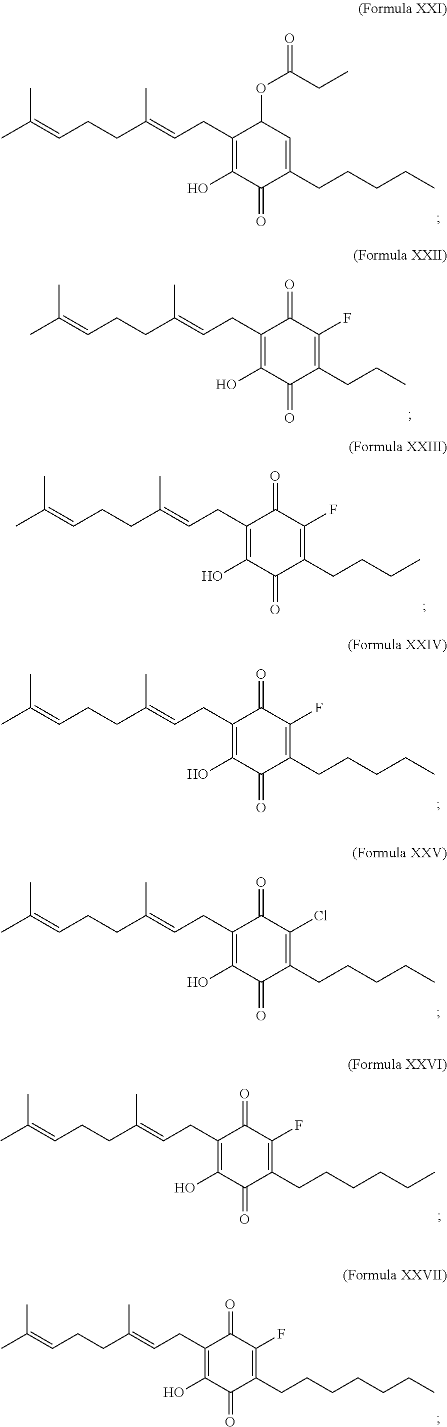

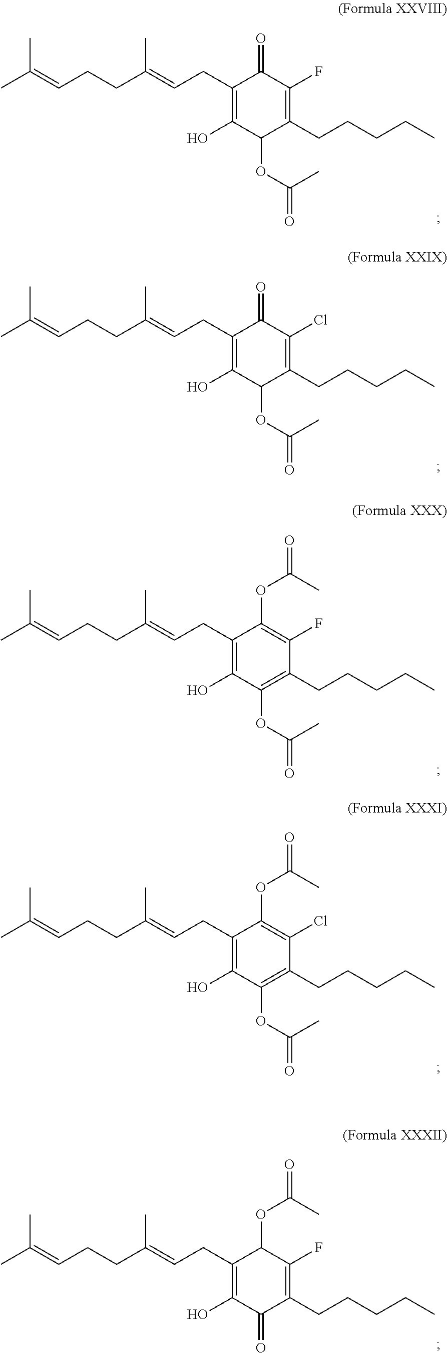

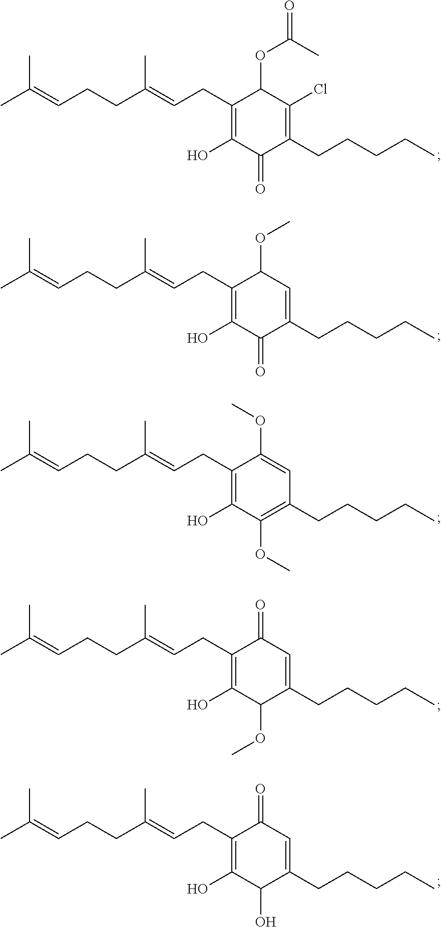

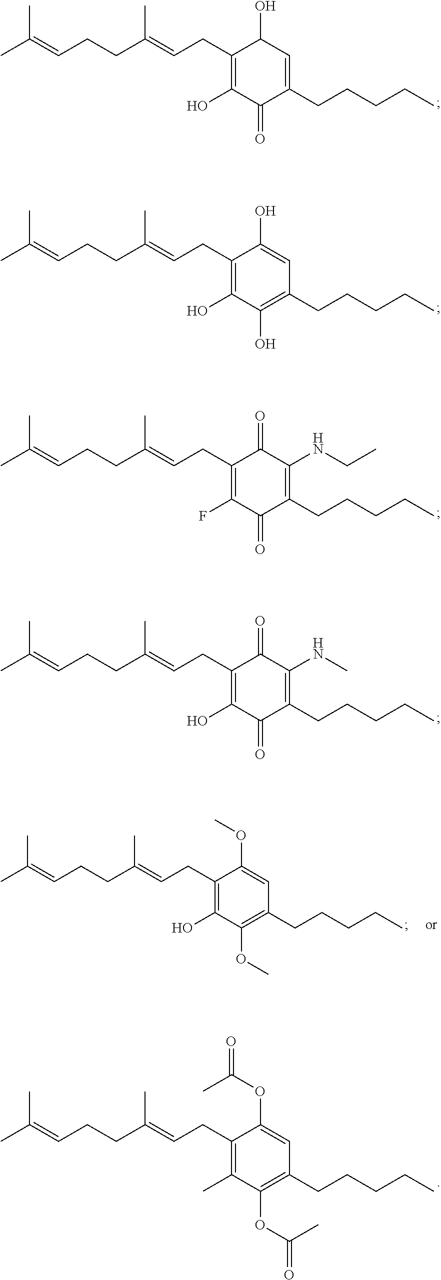

Also disclosed herein is a composition comprising at least one compound of any of the compounds of the present application, and at least one of a pharmaceutically effective carrier, a pharmaceutically effective diluent and a pharmaceutically effective excipient.

### BRIEF DESCRIPTION OF THE DRAWINGS

FIG. 1A shows a gas chromatography mass spectrometry (GC-MS) total ion chromatogram (TIC) spectra for a compound of Formula XXXIX, according to some embodiments.

FIG. 1B shows a gas chromatography mass spectrometry (GC-MS) mass-to-charge ratio (m/z) spectra for a compound of Formula XXXIX, according to some embodiments.

FIG. 1C shows a high-performance liquid chromatography ultraviolet (HPLC-UV) spectra at 254 nm for a compound of Formula XXXIX, according to some embodiments.

FIG. 1D shows a high-performance liquid chromatography evaporative light scattering detector (HPLC-ELSD) spectra for a compound of Formula XXXIX, according to some embodiments.

FIG. 2A shows a gas chromatography mass spectrometry (GC-MS) total ion chromatogram (TIC) spectra for a compound of Formula XLI, according to some embodiments.

FIG. 2B shows a gas chromatography mass spectrometry (GC-MS) mass-to-charge ratio (m/z) spectra for a compound of Formula XLI, according to some embodiments.

FIG. 2C shows a high-performance liquid chromatography ultraviolet (HPLC-UV) spectra at 254 nm for a compound of Formula XLI, according to some embodiments.

FIG. 2D shows a high-performance liquid chromatography evaporative light scattering detector (HPLC-ELSD) spectra for a compound of Formula XLI, according to some embodiments.

FIG. 3A shows a gas chromatography mass spectrometry (GC-MS) total ion chromatogram (TIC) spectra for a compound of Formula XLII, according to some embodiments.

FIG. 3B shows a gas chromatography mass spectrometry (GC-MS) mass-to-charge ratio (m/z) spectra for a compound of Formula XLII, according to some embodiments.

FIG. 3C shows a high-performance liquid chromatography evaporative light scattering detector (HPLC-ELSD) spectra for a compound of Formula XLH, according to some embodiments.

FIG. 3D shows a high-performance liquid chromatography ultraviolet (HPLC-UV) spectra at 220 nm for a compound of Formula XLII, according to some embodiments.

FIG. 4A shows a gas chromatography mass spectrometry (GC-MS) total ion chromatogram (TIC) spectra for a compound of Formula XLIII, according to some embodiments.

FIG. 4B shows a gas chromatography mass spectrometry (GC-MS) mass-to-charge ratio (m/z) spectra for a compound of Formula XLIII, according to some embodiments.

FIG. 4C shows a high-performance liquid chromatography ultraviolet (HPLC-UV) spectra at 220 nm for a compound of Formula XLIII, according to some embodiments.

FIG. 4D shows a high-performance liquid chromatography evaporative light scattering detector (HPLC-ELSD) spectra for a compound of Formula XLIII, according to some embodiments.

### DETAILED DESCRIPTION

In some embodiments, small molecule therapeutics are provided. Various embodiments of these compounds include compounds having the structure of Formula I as described herein or pharmaceutically acceptable salts thereof. In some embodiments, prodrugs, metabolites, stereoisomers, hydrates, solvates, polymorphs, and pharmaceutically acceptable salts of the compounds disclosed herein are provided.

In some embodiments, therapeutic methods or uses are provided for the treatment of inflammation using structures of Formula (I) as shown below and described herein.

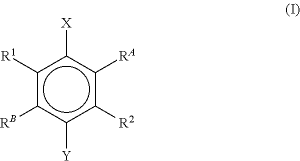

### Definitions

Unless expressly defined otherwise, technical and/or scientific terms used herein have the same meaning as is commonly understood by one of ordinary skill in the art. In the event that there are a plurality of definitions for a term herein, those in this section prevail unless stated otherwise. As used in the specification and the appended claims, the singular forms “a,” “an,” and “the” include plural referents unless the context clearly dictates otherwise. Unless otherwise indicated, conventional methods of mass spectroscopy, NMR, HPLC, protein chemistry, biochemistry, and pharmacology are employed. The use of either the conjunction “or” or “and” means “and/or” unless stated otherwise. Furthermore, use of the term “including” as well as other forms, such as “include”, “includes,” and “included,” is not limiting. As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least.” When used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

While the disclosure has been illustrated and described in detail in the foregoing description, such description is to be considered illustrative or exemplary and not restrictive. The disclosure is not limited to the disclosed embodiments. Variations to the disclosed embodiments can be understood and effected by those skilled in the art in practicing the claimed disclosure, from a study of the disclosure and the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. The mere fact that certain measures are recited in mutually different dependent claims does not indicate that a combination of these measures cannot be used to advantage.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein. It should be noted that the use of particular terminology when describing certain features or aspects of the disclosure should not be taken to imply that the terminology is being re-defined herein to be restricted to include any specific characteristics of the features or aspects of the disclosure with which that terminology is associated.

Where a range of values is provided, it is understood that the upper and lower limit, and each intervening value between the upper and lower limit of the range is encompassed within the embodiments.

Compounds disclosed herein having at least one chiral center they may exist as a racemate or as each enantiomer, and may exist as enantiomeric-enriched mixtures of the enantiomers. It should be noted that all such isomers and mixtures thereof are included in the scope of the present invention. Furthermore, the crystalline forms for the compounds disclosed herein may exist as alternative polymorphs. Such polymorphs are included in one embodiment of the present invention. In addition, some of the compounds of the present invention may form solvates with water (i.e., hydrates) or common organic solvents. Such solvates are included in one embodiment of the present invention.

The term “pharmaceutically acceptable salt,” as used herein, refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), sulfuric acid, nitric acid, phosphoric acid and the like. Pharmaceutical salts can also be obtained by reacting a compound with an organic acid such as aliphatic or aromatic carboxylic or sulfonic acids, for example acetic, succinic, lactic, malic, tartaric, citric, ascorbic, nicotinic, methanesulfonic, ethanesulfonic, p-toluensulfonic, salicylic or naphthalenesulfonic acid. Pharmaceutical salts can also be obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)-methylamine, C₁-C₇ alkylamine, cyclohexylamine, triethanolamine, ethylenediamine, and salts with amino acids such as arginine, lysine, and the like.

If the manufacture of pharmaceutical formulations involves intimate mixing of the pharmaceutical excipients and the active ingredient in its salt form, then it may be desirable to use pharmaceutical excipients which are non-basic, that is, either acidic or neutral excipients.

In various embodiments, the compounds disclosed herein can be used alone, in combination with other compounds disclosed herein, or in combination with one or more other agents active in the therapeutic areas described herein.

The term “halogen atom,” as used herein, means any one of the radio-stable atoms of column 7 of the Periodic Table of the Elements, e.g., fluorine, chlorine, bromine, or iodine, with fluorine, bromine, and chlorine being preferred.

The terms “ester” and “C-carboxy,” as used herein, refer to a “—C(═O)OR” group in which R can be the same as defined with respect to O-carboxy. An ester and C-carboxy may be substituted or unsubstituted.

A “sulfenyl” group, as used herein, refers to an “—SR” group in which R can be hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). A sulfenyl may be substituted or unsubstituted.

A “sulfinyl” group, as used herein, refers to an “—S(═O)—R” group in which R can be the same as defined with respect to sulfenyl. A sulfinyl may be substituted or unsubstituted.

A “sulfonyl” group, as used herein, refers to an “SO₂R” group in which R can be the same as defined with respect to sulfenyl. A sulfonyl may be substituted or unsubstituted.

The term “amide,” as used herein, refers to a chemical moiety with formula —(R)ₙ—C(O)NHR′ or —(R)ₙ—NHC(O)R′, where R and R′ are independently selected from the group consisting of alkyl, cycloalkyl, aryl, heteroaryl (bonded through a ring carbon) and heteroalicyclic (bonded through a ring carbon), and where n is 0 or 1. An amide may be an amino acid or a peptide molecule attached to a molecule of the present invention, thereby forming a prodrug.

Any amine, hydroxyl, or carboxyl side chain on the compounds disclosed herein can be esterified or amidified. The procedures and specific groups to be used to achieve this end are known to those of skill in the art and can readily be found in reference sources such as Greene and Wuts, Protective Groups in Organic Synthesis, 3^{rd} Ed., John Wiley & Sons, New York, NY, 1999, which is incorporated herein in its entirety.

The term “aromatic,” as used herein, refers to an aromatic group which has at least one ring having a conjugated pi electron system and includes both carbocyclic aryl (e.g., phenyl) and heterocyclic aryl groups (e.g., pyridine). The term includes monocyclic or fused-ring polycyclic (i.e., rings which share adjacent pairs of carbon atoms) groups. The term “carbocyclic” refers to a compound which contains one or more covalently closed ring structures, and that the atoms forming the backbone of the ring are all carbon atoms. The term thus distinguishes carbocyclic from heterocyclic rings in which the ring backbone contains at least one atom which is different from carbon. The term “heteroaromatic” refers to an aromatic group which contains at least one heterocyclic ring.

As used herein, “Cₐ to C_{b}” in which “a” and “b” are integers refer to the number of carbon atoms in an alkyl, alkenyl or alkynyl group, or the number of carbon atoms in the ring of a cycloalkyl, aryl, heteroaryl or heterocyclyl group. That is, the alkyl, alkenyl, alkynyl, ring of the cycloalkyl, ring of the aryl, ring of the heteroaryl or ring of the heterocyclyl can contain from “a” to “b”, inclusive, carbon atoms. Thus, for example, a “C₁ to C₄ alkyl” group or a “C₁-C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—. Likewise, for example, cycloalkyl group may contain from “a” to “b”, inclusive, total atoms, such as a C₃-C₈ cycloalkyl group, 3 to 8 carbon atoms in the ring(s). If no “a” and “b” are designated with regard to an alkyl, cycloalkyl, or cycloalkenyl, the broadest range described in these definitions is to be assumed. Similarly, a “4 to 7 membered heterocyclyl” group refers to all heterocyclyl groups with 4 to 7 total ring atoms, for example, azetidine, oxetane, oxazoline, pyrrolidine, piperidine, piperazine, morpholine, and the like. As used herein, the term “C₁-C₆” includes C₁, C₂, C₃, C₄, C₅ and C₆, and a range defined by any of the two preceding numbers. For example, C₁-C₆ alkyl includes C₁, C₂, C₃, C₄, C₅ and C₆ alkyl, C₂-C₆ alkyl, C₁-C₃ alkyl, etc. Similarly, C₃-C₈ carbocyclyl or cycloalkyl each includes hydrocarbon ring containing 3, 4, 5, 6, 7 and 8 carbon atoms, or a range defined by any of the two numbers, such as C₃-C₇ cycloalkyl or C₅-C₆ cycloalkyl. As another example, 3 to 10 membered heterocyclyl includes 3, 4, 5, 6, 7, 8, 9, or 10 ring atoms, or a range defined by any of the two preceding numbers, such as 4 to 6 membered or 5 to 7 membered heterocyclyl.

As used herein, “alkyl” refers to a straight or branched hydrocarbon chain fully saturated (no double or triple bonds) hydrocarbon group. The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 5 carbon atoms. The alkyl group of the compounds may be designated as “C₁-C₄ alkyl” or similar designations. By way of example only, “C₁-C₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, ethenyl, propenyl, butenyl, and the like.

The alkyl group may be substituted or unsubstituted. When substituted, the substituent group(s) is(are) one or more group(s) individually and independently selected from alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, ester, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, and amino, including mono- and di-substituted amino groups, and the protected derivatives thereof. Wherever a substituent is described as being “optionally substituted” that substituent may be substituted with one of the above substituents.

As used herein, “alkenyl” refers to an alkyl group that contains in the straight or branched hydrocarbon chain one or more double bonds. An alkenyl group may be unsubstituted or substituted. When substituted, the substituent(s) may be selected from the same groups disclosed above with regard to alkyl group substitution. The alkenyl group may have 2 to 20 carbon atoms, although the present definition also covers the occurrence of the term “alkenyl” where no numerical range is designated. The alkenyl group may also be a medium size alkenyl having 2 to 9 carbon atoms. The alkenyl group could also be a lower alkenyl having 2 to 4 carbon atoms. The alkenyl group of the compounds may be designated as “C₂₋₄ alkenyl” or similar designations. By way of example only, “C₂₋₄ alkenyl” indicates that there are two to four carbon atoms in the alkenyl chain, i.e., the alkenyl chain is selected from the group consisting of ethenyl, propen-1-yl, propen-2-yl, propen-3-yl, buten-1-yl, buten-2-yl, buten-3-yl, buten-4-yl, 1-methyl-propen-1-yl, 2-methyl-propen-1-yl, 1-ethyl-ethen-1-yl, 2-methyl-propen-3-yl, buta-1,3-dienyl, buta-1,2-dienyl, and buta-1,2-dien-4-yl. Typical alkenyl groups include, but are in no way limited to, ethenyl, propenyl, butenyl, pentenyl, and hexenyl, and the like.

As used herein, “alkynyl” refers to an alkyl group that contains in the straight or branched hydrocarbon chain one or more triple bonds. An alkynyl group may be unsubstituted or substituted. When substituted, the substituent(s) may be selected from the same groups disclosed above with regard to alkyl group substitution. The alkynyl group may have 2 to 20 carbon atoms, although the present definition also covers the occurrence of the term “alkynyl” where no numerical range is designated. The alkynyl group may also be a medium size alkynyl having 2 to 9 carbon atoms. The alkynyl group could also be a lower alkynyl having 2 to 4 carbon atoms. The alkynyl group of the compounds may be designated as “C₂₋₄ alkynyl” or similar designations. By way of example only, “C₂₋₃ alkynyl” indicates that there are two to four carbon atoms in the alkynyl chain, i.e., the alkynyl chain is selected from the group consisting of ethynyl, propyn-1-yl, propyn-2-yl, butyn-1-yl, butyn-3-yl, butyn-4-yl, and 2-butynyl. Typical alkynyl groups include, but are in no way limited to, ethynyl, propynyl, butynyl, pentynyl, and hexynyl, and the like.

As used herein, “heteroalkyl” refers to a straight or branched hydrocarbon chain containing one or more heteroatoms, that is, an element other than carbon, including but not limited to, nitrogen, oxygen and sulfur, in the chain backbone. The heteroalkyl group may have 1 to 20 carbon atoms although the present definition also covers the occurrence of the term “heteroalkyl” where no numerical range is designated. The heteroalkyl group may also be a medium size heteroalkyl having 1 to 9 carbon atoms. The heteroalkyl group could also be a lower heteroalkyl having 1 to 4 carbon atoms. The heteroalkyl group of the compounds may be designated as “Cia heteroalkyl” or similar designations. The heteroalkyl group may contain one or more heteroatoms. By way of example only, “C₁₋₄ heteroalkyl” indicates that there are one to four carbon atoms in the heteroalkyl chain and additionally one or more heteroatoms in the backbone of the chain.

As used herein, “aryl” refers to a carbocyclic (all carbon) ring or two or more fused rings (rings that share two adjacent carbon atoms) that have a fully delocalized pi-electron system. Examples of aryl groups include, but are not limited to, benzene, naphthalene and azulene. An aryl group may be substituted or unsubstituted. When substituted, hydrogen atoms are replaced by substituent group(s) that is(are) one or more group(s) independently selected from alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, ester, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, and amino, including mono- and di-substituted amino groups, and the protected derivatives thereof. When substituted, substituents on an aryl group may form a non-aromatic ring fused to the aryl group, including a cycloalkyl, cycloalkenyl, cycloalkynyl, and heterocyclyl.

As used herein, “heteroaryl” refers to a monocyclic or multicyclic aromatic ring system (a ring system with fully delocalized pi-electron system), one or two or more fused rings that contain(s) one or more heteroatoms, that is, an element other than carbon, including but not limited to, nitrogen, oxygen and sulfur. Examples of heteroaryl rings include, but are not limited to, furan, thiophene, phthalazine, pyrrole, oxazole, thiazole, imidazole, pyrazole, isoxazole, isothiazole, triazole, thiadiazole, pyridine, pyridazine, pyrimidine, pyrazine and triazine. A heteroaryl group may be substituted or unsubstituted. When substituted, hydrogen atoms are replaced by substituent group(s) that is(are) one or more group(s) independently selected from alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, ester, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, and amino, including mono- and di-substituted amino groups, and the protected derivatives thereof. When substituted, substituents on a heteroaryl group may form a non-aromatic ring fused to the aryl group, including a cycloalkyl, cycloalkenyl, cycloalkynyl, and heterocyclyl.

As used herein, an “aralkyl” or “arylalkyl” refers to an aryl group connected, as a substituent, via an alkylene group. The alkylene and aryl group of an aralkyl may be substituted or unsubstituted. Examples include but are not limited to benzyl, substituted benzyl, 2-phenylethyl, 3-phenylpropyl, and naphtylalkyl. In some cases, the alkylene group is a lower alkylene group.

As used herein, a “heteroaralkyl” or “heteroarylalkyl” is heteroaryl group connected, as a substituent, via an alkylene group. The alkylene and heteroaryl group of heteroaralkyl may be substituted or unsubstituted. Examples include but are not limited to 2-thienylmethyl, 3-thienylmethyl, furylmethyl, thienylethyl, pyrrolylalkyl, pyridylalkyl, isoxazollylalkyl, and imidazolylalkyl, and their substituted as well as benzo-fused analogs. In some cases, the alkylene group is a lower alkylene group.

As used herein, a “alkylene” refers to a branched, or straight chain fully saturated di-radical chemical group containing only carbon and hydrogen that is attached to the rest of the molecule via two points of attachment (i.e., an alkanediyl). The alkylene group may have 1 to 20 carbon atoms, although the present definition also covers the occurrence of the term alkylene where no numerical range is designated. The alkylene group may also be a medium size alkylene having 1 to 9 carbon atoms. The alkylene group could also be a lower alkylene having 1 to 4 carbon atoms. The alkylene group may be designated as “Cia alkylene” or similar designations. By way of example only, “C₁₋₄ alkylene” indicates that there are one to four carbon atoms in the alkylene chain, i.e., the alkylene chain is selected from the group consisting of methylene, ethylene, ethan-1,1-diyl, propylene, propan-1,1-diyl, propan-2,2-diyl, 1-methyl-ethylene, butylene, butan-1,1-diyl, butan-2,2-diyl, 2-methyl-propan-1,1-diyl, 1-methyl-propylene, 2-methyl-propylene, 1,1-dimethyl-ethylene, 1,2-dimethyl-ethylene, and 1-ethyl-ethylene.

As used herein, “alkenylene” refers to a straight or branched chain di-radical chemical group containing only carbon and hydrogen and containing at least one carbon-carbon double bond that is attached to the rest of the molecule via two points of attachment. The alkenylene group may have 2 to 20 carbon atoms, although the present definition also covers the occurrence of the term alkenylene where no numerical range is designated. The alkenylene group may also be a medium size alkenylene having 2 to 9 carbon atoms. The alkenylene group could also be a lower alkenylene having 2 to 4 carbon atoms. The alkenylene group may be designated as “C₂₋₄ alkenylene” or similar designations. By way of example only, “C₂₋₄ alkenylene” indicates that there are two to four carbon atoms in the alkenylene chain, i.e., the alkenylene chain is selected from the group consisting of ethenylene, ethen-1,1-diyl, propenylene, propen-1,1-diyl, prop-2-en-1,1-diyl, 1-methyl-ethenylene, but-1-enylene, but-2-enylene, but-1,3-dienylene, buten-1,1-diyl, but-1,3-dien-1,1-diyl, but-2-en-1,1-diyl, but-3-en-1,1-diyl, 1-methyl-prop-2-en-1,1-diyl, 2-methyl-prop-2-en-1,1-diyl, 1-ethyl-ethenylene, 1,2-dimethyl-ethenylene, 1-methyl-propenylene, 2-methyl-propenylene, 3-methyl-propenylene, 2-methyl-propen-1,1-diyl, and 2,2-dimethyl-ethen-1,1-diyl.

As used herein, “alkylidene” refers to a divalent group, such as ═CR′R″, which is attached to one carbon of another group, forming a double bond, alkylidene groups include, but are not limited to, methylidene (═CH₂) and ethylidene (═CHCH₃). As used herein, “arylalkylidene” refers to an alkylidene group in which either R′ and R″ is an aryl group. An alkylidene group may be substituted or unsubstituted.

As used herein, “alkoxy” refers to the formula —OR wherein R is an alkyl is defined as above, e.g. methoxy, ethoxy, n-propoxy, 1-methylethoxy (isopropoxy), n-butoxy, iso-butoxy, sec-butoxy, tert-butoxy, amoxy, tert-amoxy and the like. An alkoxy may be substituted or unsubstituted.

As used herein, “alkylthio” refers to the formula —SR wherein R is an alkyl is defined as above, e.g., methylmercapto, ethylmercapto, n-propylmercapto, 1-methylethylmercapto (isopropylmercapto), n-butylmercapto, iso-butylmercapto, sec-butylmercapto, tert-butylmercapto, and the like. An alkylthio may be substituted or unsubstituted.

As used herein, “aryloxy” and “arylthio” refers to RO— and RS—, respectively, in which R is an aryl, such as but not limited to phenyl. Both an aryloxyl and arylthio may be substituted or unsubstituted.

As used herein, “acyl” refers to —C(═O)R, wherein R is hydrogen, C₁₋₆ alkyl, C₂₋₆ alkenyl, C₂₋₆ alkynyl, C₃₋₇ carbocyclyl, aryl, 5-10 membered heteroaryl, and 5-10 membered heterocyclyl, as defined herein. Non-limiting examples include formyl, acetyl, propanoyl, benzoyl, and acryl.

As used herein, “cycloalkyl,” as used herein, refers to a completely saturated (no double bonds) mono- or multi-cyclic hydrocarbon ring system. When composed of two or more rings, the rings may be joined together in a fused, bridged or spiro-connected fashion. Cycloalkyl groups may range from C₃ to C₁₀, in other embodiments it may range from C₃ to C₆. A cycloalkyl group may be unsubstituted or substituted. Typical cycloalkyl groups include, but are in no way limited to, cyclopropyl, cyclobutyl, cyclopentyl, cyclohexyl, and the like. If substituted, the substituent(s) may be an alkyl or selected from those indicated above with regard to substitution of an alkyl group unless otherwise indicated. When substituted, substituents on a cycloalkyl group may form an aromatic ring fused to the cycloalkyl group, including an aryl and a heteroaryl.

As used herein, “cycloalkenyl” refers to a cycloalkyl group that contains one or more double bonds in the ring although, if there is more than one, they cannot form a fully delocalized pi-electron system in the ring (otherwise the group would be “aryl,” as defined herein). When composed of two or more rings, the rings may be connected together in a fused, bridged or spiro-connected fashion. A cycloalkenyl group may be unsubstituted or substituted. When substituted, the substituent(s) may be an alkyl or selected from the groups disclosed above with regard to alkyl group substitution unless otherwise indicated. When substituted, substituents on a cycloalkenyl group may form an aromatic ring fused to the cycloalkenyl group, including an aryl and a heteroaryl.

As used herein, “cycloalkynyl” refers to a cycloalkyl group that contains one or more triple bonds in the ring. When composed of two or more rings, the rings may be joined together in a fused, bridged or spiro-connected fashion. A cycloalkynyl group may be unsubstituted or substituted. When substituted, the substituent(s) may be an alkyl or selected from the groups disclosed above with regard to alkyl group substitution unless otherwise indicated. When substituted, substituents on a cycloalkynyl group may form an aromatic ring fused to the cycloalkynyl group, including an aryl and a heteroaryl.

As used herein, “heteroalicyclic” or “heteroalicyclyl” refers to a stable 3- to 18 membered ring which consists of carbon atoms and from one to five heteroatoms selected from the group consisting of nitrogen, oxygen and sulfur. The “heteroalicyclic” or “heteroalicyclyl” may be monocyclic, bicyclic, tricyclic, or tetracyclic ring system, which may be joined together in a fused, bridged or spiro-connected fashion; and the nitrogen, carbon and sulfur atoms in the “heteroalicyclic” or “heteroalicyclyl” may be optionally oxidized; the nitrogen may be optionally quaternized; and the rings may also contain one or more double bonds provided that they do not form a fully delocalized pi-electron system throughout all the rings. Heteroalicyclyl groups may be unsubstituted or substituted. When substituted, the substituent(s) may be one or more groups independently selected from the group consisting of alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, ester, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, haloalkyl, haloalkoxy, trihalomethanesulfonyl, trihalomethanesulfonamido, and amino, including mono- and di-substituted amino groups, and the protected derivatives thereof. Examples of such “heteroalicyclic” or “heteroalicyclyl” include but are not limited to, azepinyl, acridinyl, carbazolyl, cinnolinyl, dioxolanyl, imidazolinyl, morpholinyl, oxiranyl, piperidinyl N-oxide, piperidinyl, piperazinyl, pyrrolidinyl, 4-piperidonyl, pyrazolidinyl, 2-oxopyrrolidinyl, thiamorpholinyl, thiamorpholinyl sulfoxide, and thiamorpholinyl sulfone. When substituted, substituents on a heteroalicyclyl group may form an aromatic ring fused to the heteroalicyclyl group, including an aryl and a heteroaryl.

As used herein, the term “hydroxy” refers to a —OH group.

As used herein, “alkoxy” refers to the formula —OR wherein R is an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl) is defined herein. A non-limiting list of alkoxys is methoxy, ethoxy, n-propoxy, 1-methylethoxy (isopropoxy), n-butoxy, iso-butoxy, sec-butoxy, tert-butoxy, phenoxy and benzoxy. An alkoxy may be substituted or unsubstituted.

As used herein, the term “(cycloalkenyl)alkyl” refers to a cycloalkenyl group connected, as a substituent, via an alkylene group. The alkylene and cycloalkenyl of a (cycloalkenyl)alkyl may be substituted or unsubstituted. In some cases, the alkylene group is a lower alkylene group.

As used herein, the term “(cycloalkynyl)alkyl” to a cycloalkynyl group connected, as a substituent, via an alkylene group. The alkylene and cycloalkynyl of a (cycloalkynyl)alkyl may be substituted or unsubstituted. In some cases, the alkylene group is a lower alkylene group.

As used herein, the term “O-carboxy” refers to a “RC(═O)O—” group in which R can be hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl, as defined herein. An O-carboxy may be substituted or unsubstituted.

As used herein, the term “C-carboxy” refers to a “—C(═O)R” group in which R can be the same as defined with respect to O-carboxy. A C-carboxy may be substituted or unsubstituted.

As used herein, the term “cyano” refers to a “—CN” group.

As used herein, the term “cyanato” refers to an “—OCN” group.

As used herein, the term “isocyanato” refers to a “—NCO” group.

As used herein, the term “thiocyanato” refers to a “—SCN” group.

As used herein, the term “isothiocyanato” refers to an “—NCS” group.

As used herein, the term “sulfinyl” refers to a “—S(═O)—R” group in which R can be the same as defined with respect to O-carboxy. A sulfinyl may be substituted or unsubstituted.

As used herein, the term “sulfonyl” refers to an “—SO₂R” group in which R can be the same as defined with respect to O-carboxy. A sulfonyl may be substituted or unsubstituted.

As used herein, the term “O-carbamyl” refers to a “—OC(═O)NR_{A}R_{B}” group in which R_{A} and R_{B} can be the same as defined with respect to O-carboxy. An O-carbamyl may be substituted or unsubstituted.

As used herein, the term “N-carbamyl” refers to an “ROC(═O)NR_{A}—” group in which R and R_{A} can be the same as defined with respect to O-carboxy. An N-carbamyl may be substituted or unsubstituted.

As used herein, the term “C-amido” refers to a “—C(═O)NR_{A}R_{B}” group in which R_{A} and R_{B} can be the same as defined with respect to O-carboxy. A C-amido may be substituted or unsubstituted.

As used herein, the term “N-amido” refers to a “RC(═O)NR_{A}—” group in which R and R_{A} can be the same as defined with respect to O-carboxy. An N-amido may be substituted or unsubstituted.

As used herein, the term “amino” refers to a “—NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

A “mono-substituted amine” group refers to a “—NHR” group in which R can be an alkyl, an alkenyl, an alkynyl, a haloalkyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein. A mono-substituted amino may be substituted or unsubstituted. Examples of mono-substituted amino groups include, but are not limited to, —NH(methyl), —NH(phenyl) and the like.

As used herein, the term “aminoalkyl” refers to an amino group connected via an alkylene group.

A “di-substituted amine” group refers to a “—NR_{A}R_{B}” group in which R_{A} and R_{B} can be independently an alkyl, an alkenyl, an alkynyl, a haloalkyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein. A di-substituted amino may be substituted or unsubstituted. Examples of di-substituted amino groups include, but are not limited to, —N(methyl)₂, —N(phenyl)(methyl), —N(ethyl)(methyl) and the like.

As used herein, the term “amino acid” refers to L-amino acids. Examples of suitable L-amino acids include, but are not limited to, alanine, asparagine, aspartic acid, cysteine, glutamic acid, glutamine, glycine, proline, serine, selenocysteine, tyrosine, arginine, histidine, isoleucine, leucine, lysine, methionine, phenylalanine, threonine, tryptophan and valine. Additional examples of suitable amino acids include, but are not limited to, ornithine, hypusine, 2-aminoisobutyric acid, dehydroalanine, citrulline, alpha-ethyl-glycine, alpha-propyl-glycine and norleucine.

Where the numbers of substituents is not specified (e.g. haloalkyl), there may be one or more substituents present. For example, “haloalkyl” may include one or more of the same or different halogens. As another example, “C₁-C₃ alkoxyphenyl” may include one or more of the same or different alkoxy groups containing one, two or three atoms.

As used herein, the term “ester” refers to a “—C(═O)OR” group in which R can be the same as defined with respect to O-carboxy. An ester may be substituted or unsubstituted.

As used herein, the term “acetyl” refers to a —C(═O)CH₃, group.

As used herein, the term “O-carbamyl” refers to a —OC(═O)—NR, in which R can be hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl, as defined herein. An 0-carbamyl can be substituted or unsubstituted.

As used herein, the term “N-carbamyl” refers to a ROC(═O)NH— group, in which R can be hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl, as defined herein. An N-carbamyl can be substituted or unsubstituted.

As used herein, the term “perhaloalkyl” refers to an alkyl group where all of the hydrogen atoms are replaced by halogen atoms.

As used herein, the term “halogen” or “halo,” refer to any one of the radio-stable atoms of column 7 of the Periodic Table of the Elements, e.g., fluorine, chlorine, bromine, or iodine, with fluorine and chlorine being preferred.

As used herein, “haloalkyl” refers to an alkyl group in which one or more of the hydrogen atoms are replaced by a halogen (e.g., mono-haloalkyl, di-haloalkyl and tri-haloalkyl). Such groups include but are not limited to, chloromethyl, fluoromethyl, difluoromethyl, trifluoromethyl, 1-chloro-2-fluoromethyl and 2-fluoroisobutyl. A haloalkyl may be substituted or unsubstituted.

As used herein, “haloalkoxy” refers to an alkoxy group in which one or more of the hydrogen atoms are replaced by a halogen (e.g., mono-haloalkoxy, di-haloalkoxy and tri-haloalkoxy). Such groups include but are not limited to, chloromethoxy, fluoromethoxy, difluoromethoxy, trifluoromethoxy, 1-chloro-2-fluoromethoxy and 2-fluoroisobutoxy. A haloalkoxy may be substituted or unsubstituted.

As used herein, the term “carbocyclyl” refers to a non-aromatic cyclic ring or ring system containing only carbon atoms in the ring system backbone. When the carbocyclyl is a ring system, two or more rings may be joined together in a fused, bridged or spiro-connected fashion. Carbocyclyls may have any degree of saturation provided that at least one ring in a ring system is not aromatic. Thus, carbocyclyls include cycloalkyls, cycloalkenyls, and cycloalkynyls. The carbocyclyl group may have 3 to 20 carbon atoms, although the present definition also covers the occurrence of the term “carbocyclyl” where no numerical range is designated. The carbocyclyl group may also be a medium size carbocyclyl having 3 to 10 carbon atoms. The carbocyclyl group could also be a carbocyclyl having 3 to 6 carbon atoms. The carbocyclyl group may be designated as “C₃₋₆ carbocyclyl” or similar designations. Examples of carbocyclyl rings include, but are not limited to, cyclopropyl, cyclobutyl, cyclopentyl, cyclohexyl, cyclohexenyl, 2,3-dihydro-indene, bicycle[2.2.2]octanyl, adamantyl, and spiro[4.4]nonanyl.

As used herein, the term “(cycloalkyl)alkyl” refers to a cycloalkyl group connected, as a substituent, via an alkylene group. The alkylene and cycloalkyl of a (cycloalkyl)alkyl may be substituted or unsubstituted. Examples include but are not limited cyclopropylmethyl, cyclobutylmethyl, cyclopropylethyl, cyclopropylbutyl, cyclobutylethyl, cyclopropylisopropyl, cyclopentylmethyl, cyclopentylethyl, cyclohexylmethyl, cyclohexylethyl, cycloheptylmethyl, and the like. In some cases, the alkylene group is a lower alkylene group.

As used herein, the term “cycloalkyl” refers to a fully saturated carbocyclyl ring or ring system. Examples include cyclopropyl, cyclobutyl, cyclopentyl, and cyclohexyl.

As used herein, the term “cycloalkenyl” means a carbocyclyl ring or ring system having at least one double bond, wherein no ring in the ring system is aromatic. An example is cyclohexenyl.

As used herein, the term “heterocyclyl” refers to three-, four-, five-, six-, seven-, and eight- or more membered rings wherein carbon atoms together with from 1 to 3 heteroatoms constitute said ring. A heterocyclyl can optionally contain one or more unsaturated bonds situated in such a way, however, that an aromatic pi-electron system does not arise. The heteroatoms are independently selected from oxygen, sulfur, and nitrogen.

As used herein, the term heterocyclyl can further contain one or more carbonyl or thiocarbonyl functionalities, so as to make the definition include oxo-systems and thio-systems such as lactams, lactones, cyclic imides, cyclic thioimides, cyclic carbamates, and the like.

As used herein, “heterocyclyl” refers to a non-aromatic cyclic ring or ring system containing at least one heteroatom in the ring backbone. Heterocyclyls may be joined together in a fused, bridged or spiro-connected fashion. Heterocyclyls may have any degree of saturation provided that at least one ring in the ring system is not aromatic. The heteroatom(s) may be present in either a non-aromatic or aromatic ring in the ring system. The heterocyclyl group may have 3 to 20 ring members (i.e., the number of atoms making up the ring backbone, including carbon atoms and heteroatoms), although the present definition also covers the occurrence of the term “heterocyclyl” where no numerical range is designated. The heterocyclyl group may also be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations. In preferred six membered monocyclic heterocyclyls, the heteroatom(s) are selected from one up to three of O, N or S, and in preferred five membered monocyclic heterocyclyls, the heteroatom(s) are selected from one or two heteroatoms selected from O, N, or S. Examples of heterocyclyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline.

As used herein, the term “(heterocyclyl)alkyl” refers to a heterocyclyl group connected, as a substituent, via an alkylene group. Examples include, but are not limited to, imidazolinylmethyl and indolinylethyl.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; and use of terms like ‘preferably,’ ‘preferred,’ ‘desired,’ or ‘desirable,’ and words of similar meaning should not be understood as implying that certain features are critical, essential, or even important to the structure or function, but instead as merely intended to highlight alternative or additional features that may or cannot be utilized in a particular embodiment. In addition, the term “comprising” is to be interpreted synonymously with the phrases “having at least” or “including at least”. When used in the context of a process, the term “comprising” means that the process includes at least the recited steps, but may include additional steps. When used in the context of a compound, composition or device, the term “comprising” means that the compound, composition or device includes at least the recited features or components, but may also include additional features or components.

The terms “purified,” “substantially purified,” and “isolated” as used herein, refer to compounds disclosed herein being free of other, dissimilar compounds with which the compounds of the invention are normally associated in their natural state, so that the compounds of the invention comprise at least 0.5%, 1%, 5%, 10%, or 20%, and most preferably at least 50% or 75% of the mass, by weight, of a given sample.

Unless otherwise indicated, when a substituent is deemed to be “optionally substituted,” it is meant that the substituent” is a group that may be substituted with one or more group(s) individually and independently selected from alkyl, alkenyl, alkynyl, cycloalkyl, aryl, heteroaryl, heteroalicyclic, hydroxyl, alkoxy, aryloxy, mercapto, alkylthio, arylthio, cyano, halo, carbonyl, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, trihalomethanesulfonyl, and amino, including mono- and di-substituted amino groups, and the protected derivatives thereof. The protecting groups that may form the protective derivatives of the above substituents are known to those of skill in the art and may be found in references such as Greene and Wuts, above.

The term “agent” or “test agent,” as used herein, includes any substance, molecule, element, compound, entity, or a combination thereof. It includes, but is not limited to, e.g., protein, polypeptide, peptide or mimetic, small organic molecule, polysaccharide, polynucleotide, and the like. It can be a natural product, a synthetic compound, or a chemical compound, or a combination of two or more substances. Unless otherwise specified, the terms “agent”, “substance”, and “compound” are used interchangeably herein.

The term “analog,” as used herein, refers to a molecule that structurally resembles a reference molecule but which has been modified in a targeted and controlled manner, by replacing a specific substituent of the reference molecule with an alternate substituent. Compared to the reference molecule, an analog would be expected, by one skilled in the art, to exhibit the same, similar, or improved utility. Synthesis and screening of analogs, to identify variants of known compounds having improved characteristics (such as higher binding affinity for a target molecule) is an approach that is well known in pharmaceutical chemistry.

The term “mammal,” as used herein, has its usual biological meaning. Thus, it specifically includes, but is not limited to, primates, including simians (chimpanzees, apes, monkeys) and humans, cattle, horses, sheep, goats, swine, rabbits, dogs, cats, rats and mice but also includes many other species.

The term “microbial infection,” as used herein, refers to the invasion of the host organism, whether the organism is a vertebrate, invertebrate, fish, plant, bird, or mammal, by pathogenic microbes. This includes the excessive growth of microbes that are normally present in or on the body of a mammal or other organism. More generally, a microbial infection can be any situation in which the presence of a microbial population(s) is damaging to a host mammal. Thus, a mammal is “suffering” from a microbial infection when excessive numbers of a microbial population are present in or on a mammal's body, or when the effects of the presence of a microbial population(s) is damaging the cells or other tissue of a mammal. Specifically, this description applies to a bacterial infection. Note that the compounds of preferred embodiments are also useful in treating microbial growth or contamination of cell cultures or other media, or inanimate surfaces or objects, and nothing herein should limit the preferred embodiments only to treatment of higher organisms, except when explicitly so specified in the claims.

The term “pharmaceutically acceptable carrier” or “pharmaceutically acceptable excipient,” as used herein, includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

The term “subject,” as used herein, refers to a human or a non-human mammal, e.g., a dog, a cat, a mouse, a rat, a cow, a sheep, a pig, a goat, a non-human primate or a bird, e.g., a chicken, as well as any other vertebrate or invertebrate.

The term “effective amount” or a “therapeutically effective amount,” as used herein, refers to an amount of a therapeutic agent that is effective to relieve, to some extent, or to reduce the likelihood of onset of, one or more of the symptoms of a disease or condition, and includes curing a disease or condition. “Curing” means that the symptoms of a disease or condition are eliminated; however, certain long-term or permanent effects may exist even after a cure is obtained (such as extensive tissue damage).

As used herein, the terms “treat,” “treatment,” “treating,” or “amelioration” refer to therapeutic treatments, wherein the object is to reverse, alleviate, ameliorate, inhibit, slow down or stop the progression or severity of a condition, e.g., a chronic inflammatory condition, associated with a disease or disorder, e.g. rheumatoid arthritis, Crohn's disease, etc. The term “treating” includes reducing or alleviating at least one adverse effect or symptom of a condition, disease or disorder associated with, e.g., rheumatoid arthritis, Crohn's disease, etc. Treatment is generally “effective” if one or more local or systemic conditions, symptoms or clinical biomarkers of disease are reduced. Alternatively, treatment is “effective” if the progression of a disease is reduced or halted. That is, “treatment” includes not just the improvement of symptoms or biomarkers, but also a cessation of, or at least slowing of, progress or worsening of symptoms compared to what would be expected in the absence of treatment. Thus, a treatment is considered effective if one or more of the signs or symptoms of a condition described herein are altered in a beneficial manner, other clinically accepted symptoms are improved, or even ameliorated and/or reversed back to a more normal or normal state, or a desired response is induced e.g., by at least 10% following treatment according to the methods described herein. Beneficial or desired clinical results include, but are not limited to, alleviation of one or more symptom(s), diminishment of extent of disease, e.g., chronic inflammatory disease, stabilized (e.g., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject. Delivery and/or placement options include any suitable medicament delivery systems for intraoral, interproximal, intrasulcular, intra-periodontal pocket, intracanal, and intranasal. In some embodiments, a suitable delivery option includes any suitable mechanical and automated dental and medical syringes, including all calibrated and non-calibrated, all attachments, and all designs of tips including but not limited to blunt ended, and side port; Medicament delivery trays and systems including PerioProtect Trays; Medicament applicator delivery systems; Slow releasing medical preparation for intrasulcular drug delivery; Filler, oral packing, fiber, microparticles, films, gels, injectable gels, vesicular systems, strips compacts, chip, hydrogel, thermal gel, liquid, solid, including Actisite, Arestin, Atridox, Ossix Plus, Periochip, Periostat, Periofil; Injectable systems; Professional irrigation systems including piezoelectric and ultrasonic cavitron units with and without reservoir including Ora-Tec Viajet and Oral irrigation systems including Interplak, Waterpik, Hydrofloss, Viajet, Airfloss and Pro.

It is to be understood that where compounds disclosed herein have unfilled valencies, then the valencies are to be filled with hydrogens and/or deuteriums.

It is understood that the compounds described herein can be labeled isotopically or by another other means, including, but not limited to, the use of chromophores or fluorescent moieties, bioluminescent labels, or chemiluminescent labels. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium), hydrogen-2 (deuterium), and hydrogen-3 (tritium). Thus, reference herein to a compound encompasses all potential isotopic forms unless the context clearly dictates otherwise.

The term “about,” as used herein, refers to a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length. When a value is preceded by the term about, the component is not intended to be limited strictly to that value, but it is intended to include amounts that vary from the value.

The term “IC50,” as used herein, means the half maximal inhibitory concentration. That is, the dose of a particular compound or substance that inhibits a biological process by 50%. Non-limiting examples of inhibition by administration of a compound include the inhibition of protein and/or receptor activity and loss of cell viability. The term “EC50,” as used herein, means the half maximal effective concentration. That is, the dose of a particular compound or substance that causes half-maximum effect. It will be understood that in the case where the effect of a compound or substance is the inhibition of a biological process, the term “EC50” can be used interchangeably with the term “IC50.”

### Compounds

Some embodiments provide a compound of Formula (I), or a pharmaceutically acceptable salt thereof, having the structure:

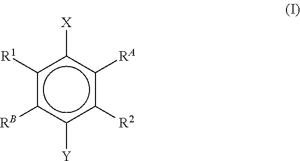

In some embodiments, each of R^{A} and R^{B} is independently selected from the group consisting of: —H, —F, —Cl, —Br, —I, —CH₃, —CH₂CH₃, —CF₃, —OH, —OCH₃, —OCH₂CH₃, —O(CO)NH₂, —O(CO)CH₃, —O(CO)CH₂CH₃, —CN, —NH₂, —NHCH₂CH₂CH₃, —NHCH₂CH₃, —N(CH₃)₂ and —NHCH(CH₃)₂; each of X and Y is independently selected from the group consisting of: —OH, ═O, —OCH₃, —O(CO)CH₃ and —O(CO)CH₂CH₃; R¹ is a moiety of formula (II):

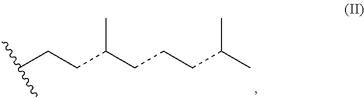

wherein each dashed bond of R1 is individually selected from representing a carbon-carbon single bond and representing a carbon-carbon double bond, and R2 is either a moiety of the formula (III-A) of —(CH2)nCH3, wherein n in an integer greater than one and less than seven, or a moiety of the formula (III-B) or —(CH2)m(CH═CH)(CH2)pCH3, wherein m is an integer greater than zero and less than four, and wherein p is an integer greater than or equal to zero and less than four, and wherein the sum of m and p is not greater than four.

In some embodiments, R^{A} is selected from the group consisting of: —H, —F, —Cl, —Br, —I, —CH₃, —CH₂CH₃, —CF₃, —OH, —OCH₃, —OCH₂CH₃, —O(CO)NH₂, —CN, —NH₂, —NHCH₂CH₂CH₃, —NHCH₂CH₃, —N(CH₃)₂ and —NHCH(CH₃)₂; R^{a} is selected from the group consisting of —H, —Cl, —Br, —I, —CH₃, —CH₂CH₃, —CF₃, —OH, —OCH₂CH₃, —OCONH₂, —O(CO)CH₃, —O(CO)CH₂CH₃—CN, —NH₂, —NHCH₂CH₂CH₃, —NHCH₂CH₃, —N(CH₃)₂ and —NHCH(CH₃)₂; each of X and Y is independently selected from the group consisting of: —OH, ═O, —OCH₃, —OCOCH₃ and —O(CO)CH₂CH₃; R¹ is a moiety of formula (II):

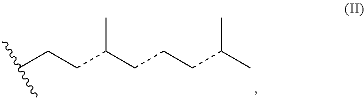

wherein each dashed bond of R¹ is individually selected from representing a carbon-carbon single bond and representing a carbon-carbon double bond, and R² is either a moiety of the formula (III-A) of —(CH₂)ₙCH₃, wherein n in an integer greater than one and less than seven, or a moiety of the formula (III-B) or —(CH₂)ₘ(CH═CH)(CH₂)ₚCH₃, wherein m is an integer greater than zero and less than four, and wherein p is an integer greater than or equal to zero and less than four, and wherein the sum of m and p is not greater than four.

In some embodiments, R^{A} is selected from the group consisting of: —H, —F, —Cl, —Br, —I, —CH₃, —CH₂CH₃, —CF₃, —OH, —OCH₃, —OCH₂CH₃, —O(CO)NH₂, —O(CO)CH₃, —O(CO)CH₂CH₃, —CN, —NH₂, —NHCH₂CH₂CH₃, —NHCH₂CH₃, —N(CH₃)₂ and —NHCH(CH₃)₂. In some further embodiments, R^{A} is —H. In some further embodiments, R^{A} is selected from the group consisting of —F, —Cl, and —Br. In some further embodiments of the compound of formula (I), R^{A} is —OH. In some further embodiments, R^{A} is selected from the group consisting of —OCH₃, —OCH₂CH₃, —O(CO)CH₃, —O(CO)CH₂CH₃, and —NH₂. In some embodiments, R^{A} is selected from the group consisting of —¹H, —²H, —³H, —CH₃, —COOH, CH(OH)₂, —CHOOH, —OCOOH, —OC(OH)₂, —C(OH)₃, —OCO₂, —CH₂O, —CHO, —OCHO, —OCH₂OH, —CH₂OH, —CH₂CHO, —CH₂OCH₃, —CHOCH₃, —CHOCH₂CH₃, —CO(O)CHO, —OCHCHO, —OCOCHO, —CH₂CH₂OH, —C(O)OCH₂OH, —OCH₂CH₂OH, —OC(O)OCH₂OH, —OCH₂COOH, —OC(O)OCOOH, —CH₂CH₂CHO, —CH₂CH₂CH₂OH, —CH₂CH₂COOH, —OCH₂CH₂CHO, —OCH₂CH₂CH₂OH, —OCH₂CH₂COOH, —CH₂C(O)OCHO, —CH₂C(O)OCH₂OH, —CH₂C(O)OCOOH, —C(O)OCH₂CHO, —C(O)OCH₂CH₂OH, —C(O)OCH₂COOH, —OC(O)CH₂CHO, —OC(O)CH₂CH₂OH, —OC(O)CH₂COOH, —OCHOCHCHO, —OCHOCHCH₂OH, —OCHOCHOOH, —NO, —NO₂, —NOH, N(OH)₂, —NH₂, —NHOH, —ONH₂, —ONH, —ONOH, —NHCOOH, —NHCH(OH)₂, —NHC(OH)₃, —NHCHO, —NHCO, —NHCH₂OH, —NHCH₂OH, —NHCH₂CHO, —NHC(O)OCHO, —NHCH₂CHO, —NHCOCHO, —NHCH₂CH₂OH, —NHC(O)OCH₂OH, —NHCH₂COOH, —NHC(O)OCOOH, —NHCH₂CH₂CHO, —NHCH₂CH₂CH₂OH, —NHCH₂CH₂COOH, —NHCH₂C(O)OCHO, —NHCH₂C(O)OCH₂OH, —NHCH₂C(O)OCOOH, —NHC(O)OCH₂CHO, —NHC(O)OCH₂CH₂OH, —NHC(O)OCH₂COOH, —NHCHOCHCHO, —NHCHOCHCH₂OH, and —NHCHOCHOOH.

In some embodiments, R^{B} is selected from the group consisting of: —H, —F, —Cl, —Br, —I, —CH₃, —CH₂CH₃, —CF₃, —OH, —OCH₃, —OCH₂CH₃, —O(CO)NH₂, —O(CO)CH₃, —O(CO)CH₂CH₃, —CN, —NH₂, —NHCH₂CH₂CH₃, —NHCH₂CH₃, —N(CH₃)₂ and —NHCH(CH₃)₂. In some further embodiments of the compound of formula (I), R^{B} is —OH. In some further embodiments, R^{B} is selected from the group consisting of —OCH₃, —OCH₂CH₃, —O(CO)CH₃, —O(CO)CH₂CH₃, and —NH₂. In some further embodiments, R^{B} is —H. In some further embodiments, R^{B} is selected from the group consisting of —F, —Cl, and —Br. In some embodiments, R^{B} is selected from the group consisting of: —¹H, —²H, —³H, —CH₃, —COOH, —CH(OH)₂, —CHOOH, —OCOOH, —OC(OH)₂, —C(OH)₃, —OCO₂, —CH₂O, —CHO, OCHO, —OCH₂OH, —CH₂OH, —CH₂CHO, —CH₂OCH₃, —CHOCH₃, —CHOCH₂CH, —CO(O)CHO, —OCHCHO, —OCOCHO, —CH₂CH₂OH, —C(O)OCH₂OH, —OCH₂CH₂OH, —OC(O)OCH₂OH, —OCH₂COOH, —OC(O)OCOOH, —CH₂CH₂CHO, —CH₂CH₂CH₂OH, —CH₂CH₂COOH, —OCH₂CH₂CHO, —OCH₂CH₂CH₂OH, —OCH₂CH₂COOH, —CH₂C(O)OCHO, —CH₂C(O)OCH₂OH, —CH₂C(O)OCOOH, —C(O)OCH₂CHO, —C(O)OCH₂CH₂OH, —C(O)OCH₂COOH, —OC(O)CH₂CHO, —OC(O)CH₂CH₂OH, —OC(O)CH₂COOH, —OCHOCHCHO, —OCHOCHCH₂OH, —OCHOCHOOH, —NO, —NO₂, —NOH, N(OH)₂, —NH₂, —NHOH, —ONH₂, —ONH, —ONOH, —NHCOOH, —NHCH(OH)₂, —NHC(OH)₃, —NHCHO, —NHCO, —NHCH₂OH, —NHCH₂OH, —NHCH₂CHO, —NHC(O)OCHO, —NHCH₂CHO, —NHCOCHO, —NHCH₂CH₂OH, —NHC(O)OCH₂OH, —NHCH₂COOH, —NHC(O)OCOOH, —NHCH₂CH₂CHO, —NHCH₂CH₂CH₂OH, —NHCH₂CH₂COOH, —NHCH₂C(O)OCHO, —NHCH₂C(O)OCH₂OH, —NHCH₂C(O)OCOOH, —NHC(O)OCH₂CHO, —NHC(O)OCH₂CH₂OH, —NHC(O)OCH₂COOH, —NHCHOCHCHO, —NHCHOCHCH₂OH, and —NHCHOCHOOH.

In some embodiments, each of X and Y is independently selected from the group consisting of: —OH, ═O, —OCH₃, —O(CO)CH₃ and —O(CO)CH₂CH₃. In some further embodiments, each of X and Y is ═O. In some further embodiments, each of X and Y is independently selected from the group consisting of —OCOCH₃ and —O(CO)CH₂CH₃. In some embodiments, X is selected from the group consisting of: —OH, ═O, —OCH₃, —O(CO)CH₃ and —O(CO)CH₂CH₃. In some further embodiments, X is ═O. In some further embodiments, X is selected from the group consisting of —OCOCH₃ and O(CO)CH₂CH₃. In some embodiments, X is selected from the group consisting of —¹H, ²H, —³H, —COOH, —CHO, —CH₂OH, —OCOOH, —OCHO, —OCH₂OH, —CH₃, —CH₂OCH₃, CHOCH₃, —CHOCH₂CH₃, —CH₂O, —CH₂CH₂OH, —OCH₂CH₂OH, —OCH₂COOH, —OCH₂CH₂CHO, —OCH₂CH₂CH₂OH, —NHCHO, —NHCH₂OH, —NHCH₂CHO, —NHCH₂CH₂CH₂OH, NHCH₂CH₂CHO, —CHOOH, —OC(OH)₂, —C(OH)₃, —OCO₂, —CH₂CHO, —CO(O)CHO, —OCHCHO, —OCOCHO, —C(O)OCH₂OH, —OC(O)OCH₂OH, —OC(O)OCOOH, —CH₂CH₂CHO, —CH₂CH₂CH₂OH, —CH₂CH₂COOH, —OCH₂CH₂COOH, —CH₂C(O)OCHO, —CH₂C(O)OCH₂OH, —CH₂C(O)OCOOH, —C(O)OCH₂CHO, —C(O)OCH₂CH₂OH, —C(O)OCH₂COOH, —OC(O)CH₂CHO, —OC(O)CH₂CH₂OH, —OC(O)CH₂COOH, —OCHOCHCHO, —OCHOCHCH₂OH, —OCHOCHOOH, —NO, —N02, —NOH, N(OH)₂. —NH₂, —NHOH, —ONH₂, —ONH, —ONOH, —NHCOOH, —NHCH(OH)₂, —NHC(OH)₃, —NHCO, —NHCH₂OH, —NHC(O)OCHO, —NHCH₂CHO, —NHCOCHO, —NHCH₂CH₂OH, —NHC(O)OCH₂OH, —NHCH₂COOH, —NHC(O)OCOOH, —NHCH₂CH₂COOH, —NHCH₂C(O)OCHO, —NHCH₂C(O)OCH₂OH, —NHCH₂C(O)OCOOH, —NHC(O)OCH₂CHO, —NHC(O)OCH₂CH₂OH, —NHC(O)OCH₂COOH, —NHCHOCHCHO, —NHCHOCHCH₂OH, and —NHCHOCHOOH.

In some embodiments, Y is selected from the group consisting of: —OH, ═O, —OCH₃, —O(CO)CH₃ and —O(CO)CH₂CH₃. In some further embodiments, Y is ═O. In some further embodiments, Y is selected from the group consisting of —OCOCH₃ and —O(CO)CH₂CH₃. In some embodiments, Y is —¹H, —²H, —³H, —COOH, —CHO, —CH₂OH, —OCOOH, —OCHO, —OCH₂OH, —CH₃, —CH₂OCH₃, —CHOCH₃, —CHOCH₂CH₃, —CH₂O, —CH₂CH₂OH, —OCH₂CH₂OH, —OCH₂COOH, —OCH₂CH₂CHO, —OCH₂CH₂CH₂OH, —NHCHO, —NHCH₂OH, —NHCH₂CHO, —NHCH₂CH₂CH₂OH, —NHCH₂CH₂CHO, —CHOOH, —OC(OH)₂, —C(OH)₃, —OCO₂, —CH₂CHO, —CO(O)CHO, —OCHCHO, —OCOCHO, —C(O)OCH₂OH, —OC(O)OCH₂OH, —OC(O)OCOOH, —CH₂CH₂CHO, —CH₂CH₂CH₂OH, —CH₂CH₂COOH, —OCH₂CH₂COOH, —CH₂C(O)OCHO, —CH₂C(O)OCH₂OH, —CH₂C(O)OCOOH, —C(O)OCH₂CHO, —C(O)OCH₂CH₂OH, —C(O)OCH₂COOH, —OC(O)CH₂CHO, —OC(O)CH₂CH₂OH, —OC(O)CH₂COOH, —OCHOCHCHO, —OCHOCHCH₂OH, —OCHOCHOOH, —NO, —NO₂, —NOH, —N(OH)₂. —NH₂, —NHOH, —ONH₂, —ONH, —ONOH, —NHCOOH, —NHCH(OH)₂, —NHC(OH)₃, —NHCO, —NHCH₂OH, —NHC(O)OCHO, —NHCH₂CHO, —NHCOCHO, —NHCH₂CH₂OH, —NHC(O)OCH₂OH, —NHCH₂COOH, —NHC(O)OCOOH, —NHCH₂CH₂COOH, —NHCH₂C(O)OCHO, —NHCH₂C(O)OCH₂OH, —NHCH₂C(O)OCOOH, —NHC(O)OCH₂CHO, —NHC(O)OCH₂CH₂OH, —NHC(O)OCH₂COOH, —NHCHOCHCHO, —NHCHOCHCH₂OH, and —NHCHOCHOOH.

In some embodiments, R¹ is a moiety of formula (II):

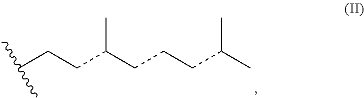

wherein each dashed bond of R¹ is individually selected from representing a carbon-carbon single bond and representing a carbon-carbon double bond. In some embodiments, each dashed bond of R¹ is a single bond. In some embodiments, each dashed bond of R¹ is a double bond. In some embodiments, R¹ is a moiety selected from the group consisting of formulas (IIa-IIf)

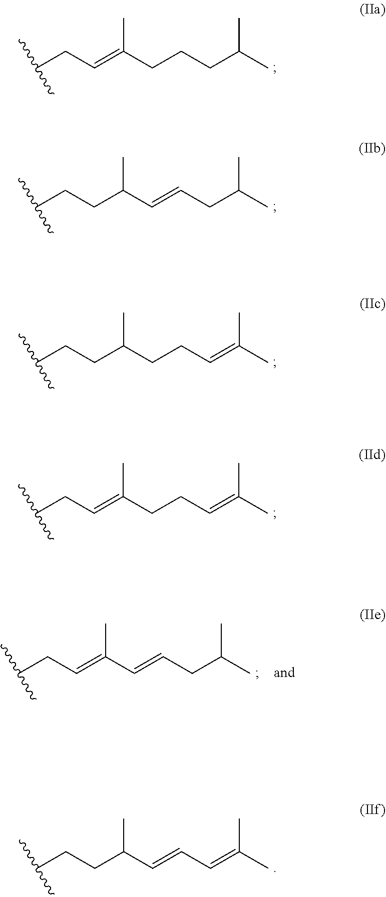 [SMILES: *C/C=C(\C)CCCC(C)C ; *CCC(C)/C=C/CC(C)C ; *CCC(C)CCC=C(C)C ; *C/C=C(\C)CCC=C(C)C ; *C/C=C(C)/C=C/CC(C)C ; *CCC(C)/C=C/C=C(C)C]

In some embodiments, R² is a carbon moiety liked with single covalent bonds. In some embodiments, R² is a carbon moiety with one double bond. In some embodiments, R² is a carbon moiety with two double bonds. In some embodiments, R² is a carbon moiety with three double bonds. In some embodiments, R² is a carbon moiety with four double bonds. In some embodiments, R² is a carbon moiety with five double bonds. In some embodiments, R² is a carbon moiety with six double bonds. In some embodiments, R² is a carbon moiety with seven double bonds. In some embodiments, R² is either a moiety of the formula (III-A) of —(CH₂)ₙCH₃, wherein n in an integer greater than one and less than seven, or a moiety of the formula (III-B) of —(CH₂)ₘ(CH═CH)(CH₂)ₚCH₃, wherein m is an integer greater than zero and less than four, and wherein p is an integer greater than or equal to zero and less than four, and wherein the sum of m and p is not greater than four. In some embodiments, R² is a three-carbon moiety. In some embodiments, R² is a four-carbon moiety. In some embodiments, R² is a five-carbon moiety. In some embodiments, R² is a six-carbon moiety. In some embodiments, R² is a seven-carbon moiety. In some embodiments, R² is a moiety selected from the group consisting of formulas (IIIa-IIIz): —(CH₂)₂CH₃ (IIa); —(CH₂)₃CH₃ (IIIb); —(CH₂)₄CH₃ (IIIc); —(CH₂)₅CH₃ (IIId); —(CH₂)₆CH₃ (IIIe); —(CH₂)₇CH₃ (IIIf); —(CH═CH)CH₃ (IIIg); —(CH═CH)CH₂CH₃ (IIIh); —(CH═CH)(CH₂)₂CH₃ (IIIi); —(CH═CH)(CH₂)₃CH₃ (IIIj); —(CH═CH)(CH₂)₃CH₃ (IIIk); —CH₂CH═CH₂ (IIIl); —CH₂(CH═CH)CH₃ (IIIm); —(CH₂)(CH═CH)(CH₂)CH₃ (IIIn); —(CH₂)(CH═CH)(CH₂)₂CH₃ (IIIo); —(CH₂)(CH═CH)(CH₂)₃CH₃ (IIIp); —(CH₂)₂CH═CH₂ (IIIq); —(CH₂)₂(CH═CH)CH₃ (IIIr); —(CH₂)₂(CH═CH)(CH₂)CH₃ (IIIs); —(CH₂)₂(CH═CH)(CH₂)₂CH₃ (IIIt), —(CH₂)₃CH═CH₂ (IIIu), —(CH₂)₃(CH═CH)CH₃ (IIIv); —(CH₂)₃(CH═CH)CH₂CH₃ (IIIw); —(CH₂)₄CH═CH₂ (IIIx); —(CH₂)₄(CH═CH)CH₃ (IIIy); and —(CH₂)₅CH═CH₂ (IIIz).

In some embodiments, the compound is selected from the group consisting of formula (IV-VIII). In some embodiments, the compound is of formula VI. In some embodiments, the compound is within the scope of an asserted genus of compounds, but with the caveat that it is not one or more or any of the compounds selected from the group consisting of formula (IV-VIII).

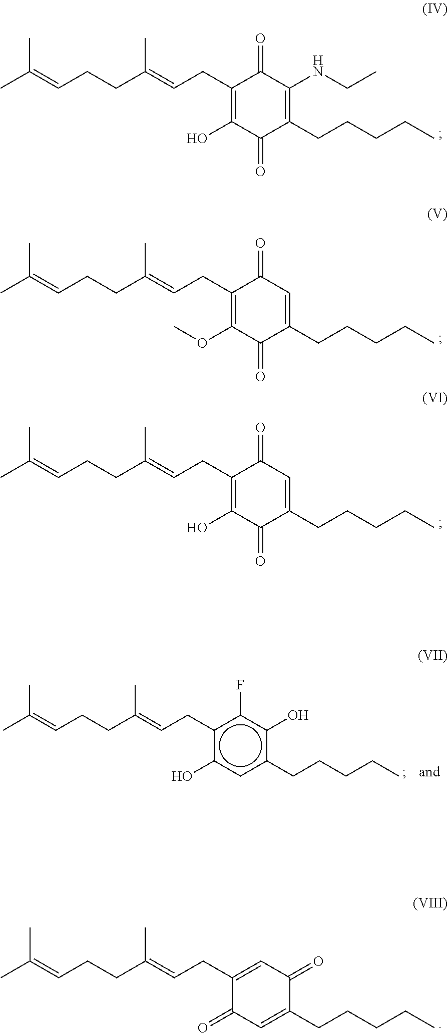

In some embodiments, if R^{B} is —OH, then R^{A} is neither —H nor —NHCH₂CH₃. In some embodiments, R^{A} and R^{B} are not both —H.

In some embodiments, the compound is formulated for intradermal, topical, transdermal, oral, buccal, sublingual, nasal or intravenous application. In some embodiments, the compound is combined with an at least one of a pharmaceutically effective carrier, a pharmaceutically effective diluent and a pharmaceutically effective excipient.

In some embodiments, the compound inhibits the activity and/or expression of one or more of TNF, IL-6, IL-1β, IL-10, IFNg, Lox5, CB2 and/or Lox15. In some embodiments, the compound inhibits an activity selected from the group consisting of TNF, IL-6, IL-1p, IFNg, Lox5, Lox15, IL-10, CB2, and combinations thereof with an EC50 that is at most about 20 μM. In some embodiments, the compound has at least about 100-fold decreased toxicity to cells relative to staurosporine. In some embodiments, the compound has at least about 2 fold selectivity for inhibiting the CB2 receptor over the CB1 receptor. In some embodiments, the compound inhibits TNF activity with an EC50 that is at most about 20 μM. In some embodiments, the compound inhibits IL-6 activity with an EC50 that is at most about 20 μM. In some embodiments, the compound inhibits IL-1β activity with an EC50 that is at most about 10 μM. In some embodiments, the compound inhibits IFNg activity with an EC50 that is at most about 1 μM. In some embodiments, the compound inhibits Lox5 activity with an EC50 that is at most about 0.3 μM. In some embodiments, the compound inhibits Lox15 activity with an EC50 that is at most about 1 μM.

In some embodiments, the compound has therapeutic activity against one or more of systemic inflammation, acute inflammation, chronic inflammation, developmental inflammation, meta-inflammation, and inflammaging.

### Pharmaceutical Compositions

In some embodiments, any of the compounds disclosed herein may be further formulated for intradermal, topical, transdermal, oral, buccal, sublingual, nasal or intravenous application. In some embodiments, any of the compounds disclosed herein may be further combined with an at least one of a pharmaceutically effective carrier, a pharmaceutically effective diluent and a pharmaceutically effective excipient.

In another aspect, pharmaceutical compositions are disclosed that comprise a physiologically acceptable surface active agents, carriers, diluents, excipients, smoothing agents, suspension agents, film forming substances, and coating assistants, or a combination thereof; and a compound disclosed herein. Acceptable carriers or diluents for therapeutic use are well known in the pharmaceutical art, and are described, for example, in Remington's Pharmaceutical Sciences, 18th Ed., Mack Publishing Co., Easton, PA (1990), which is incorporated herein by reference in its entirety. Preservatives, stabilizers, dyes, sweeteners, fragrances, flavoring agents, and the like may be provided in the pharmaceutical composition. For example, sodium benzoate, ascorbic acid and esters of p-hydroxybenzoic acid may be added as preservatives. In addition, antioxidants and suspending agents may be used. In various embodiments, alcohols, esters, sulfated aliphatic alcohols, and the like may be used as surface active agents; sucrose, glucose, lactose, starch, crystallized cellulose, mannitol, light anhydrous silicate, magnesium aluminate, magnesium methasilicate aluminate, synthetic aluminum silicate, calcium carbonate, sodium acid carbonate, calcium hydrogen phosphate, calcium carboxymethyl cellulose, and the like may be used as excipients; magnesium stearate, talc, hardened oil and the like may be used as smoothing agents; coconut oil, olive oil, sesame oil, peanut oil, soya may be used as suspension agents or lubricants; cellulose acetate phthalate as a derivative of a carbohydrate such as cellulose or sugar, or methylacetate-methacrylate copolymer as a derivative of polyvinyl may be used as suspension agents; and plasticizers such as ester phthalates and the like may be used as suspension agents.

The term “pharmaceutical composition,” as used herein, refers to a mixture of a compound disclosed herein with other chemical components, such as diluents or carriers. The pharmaceutical composition facilitates administration of the compound to an organism. Multiple techniques of administering a compound exist in the art including, but not limited to, oral, injection, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

The term “carrier,” as used herein, refers to a chemical compound that facilitates the incorporation of a compound into cells, tissues, organs, or organs systems. For example dimethyl sulfoxide (DMSO) is a commonly utilized carrier as it facilitates the uptake of many organic compounds into the cells or tissues of an organism.

The term “diluent,” as used herein, refers to chemical compounds diluted in water that will dissolve the compound of interest as well as stabilize the biologically active form of the compound. Salts dissolved in buffered solutions are utilized as diluents in the art. One commonly used buffered solution is phosphate buffered saline because it mimics the salt conditions of human blood. Since buffer salts can control the pH of a solution at low concentrations, a buffered diluent rarely modifies the biological activity of a compound.

The term “physiologically acceptable,” as used herein, refers to a carrier or diluent that does not abrogate the biological activity and properties of the compound.

As used herein, an “excipient” refers to an inert substance that is added to a pharmaceutical composition to provide, without limitation, bulk, consistency, stability, binding ability, lubrication, disintegrating ability etc., to the composition. A “diluent” is a type of excipient.

For each of the compounds described herein, and for each genus or sub-genus of compounds described herein, also described are pharmaceutical compositions comprising the compound, alone or in a mixture with other compounds of the genus or sub-genus, or with alternative compounds described herein, or with one or more alternative pharmaceutically active compounds, and one or more pharmaceutically acceptable carrier, diluent, excipient or combination thereof. The pharmaceutical compositions described herein can be administered to a human patient per se, or in pharmaceutical compositions where they are mixed with other active ingredients, as in combination therapy, or carriers, diluents, excipients or combinations thereof. Proper formulation is dependent upon the route of administration chosen. Techniques for formulation and administration of the compounds described herein are known to those skilled in the art.

The pharmaceutical compositions disclosed herein may be manufactured in any manner that is itself known, e.g., by means of conventional mixing, dissolving, granulating, dragee-making, levigating, emulsifying, encapsulating, entrapping or tableting processes. Additionally, the active ingredients are contained in an amount effective to achieve its intended purpose. Many of the compounds used in the pharmaceutical combinations disclosed herein may be provided as salts with pharmaceutically compatible counterions.

The pharmaceutical compositions described herein can be administered to a human patient per se, or in pharmaceutical compositions where they are mixed with other active ingredients, as in combination therapy, or suitable carriers or excipient(s). Techniques for formulation and administration of the compounds of the instant application may be found in “Remington's Pharmaceutical Sciences,” Mack Publishing Co., Easton, PA, 18th edition, 1990.

Suitable routes of administration may, for example, include oral, rectal, transmucosal, topical, or intestinal administration; parenteral delivery, including intramuscular, subcutaneous, intravenous, intramedullary injections, as well as intrathecal, direct intraventricular, intraperitoneal, intranasal, or intraocular injections. The compounds can also be administered in sustained or controlled release dosage forms, including depot injections, osmotic pumps, pills, transdermal (including electrotransport) patches, and the like, for prolonged and/or timed, pulsed administration at a predetermined rate.

The pharmaceutical compositions of the present invention may be manufactured in a manner that is itself known, e.g., by means of conventional mixing, dissolving, granulating, dragee-making, levigating, emulsifying, encapsulating, entrapping or tabletting processes.

Pharmaceutical compositions for use in accordance with the present invention thus may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art; e.g., in Remington's Pharmaceutical Sciences, above.

Injectables can be prepared in conventional forms, either as liquid solutions or suspensions, solid forms suitable for solution or suspension in liquid prior to injection, or as emulsions. Suitable excipients are, for example, water, saline, dextrose, mannitol, lactose, lecithin, albumin, sodium glutamate, cysteine hydrochloride, and the like. In addition, if desired, the injectable pharmaceutical compositions may contain minor amounts of nontoxic auxiliary substances, such as wetting agents, pH buffering agents, and the like. Physiologically compatible buffers include, but are not limited to, Hanks's solution, Ringer's solution, or physiological saline buffer. If desired, absorption enhancing preparations (for example, liposomes), may be utilized.

For transmucosal administration, penetrants appropriate to the barrier to be permeated may be used in the formulation.

Pharmaceutical formulations for parenteral administration, e.g., by bolus injection or continuous infusion, include aqueous solutions of the active compounds in water-soluble form. Additionally, suspensions of the active compounds may be prepared as appropriate oily injection suspensions. Suitable lipophilic solvents or vehicles include fatty oils such as sesame oil, or other organic oils such as soybean, grapefruit or almond oils, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

Further disclosed herein are various pharmaceutical compositions well known in the pharmaceutical art for uses that include intraocular, intranasal, and intraauricular delivery. Suitable penetrants for these uses are generally known in the art. Pharmaceutical compositions for intraocular delivery include aqueous ophthalmic solutions of the active compounds in water-soluble form, such as eyedrops, or in gellan gum (Shedden et al., Clin. Ther., 23(3):440-50 (2001)) or hydrogels (Mayer et al., Ophthalmologica, 210(2):101-3 (1996)); ophthalmic ointments; ophthalmic suspensions, such as microparticulates, drug-containing small polymeric particles that are suspended in a liquid carrier medium (Joshi, A., J. Ocul. Pharmacol., 10(1):29-45 (1994)), lipid-soluble formulations (Alm et al., Prog. Clin. Biol. Res., 312:447-58 (1989)), and microspheres (Mordenti, Toxicol. Sci., 52(1):101-6 (1999)); and ocular inserts. All of the above-mentioned references, are incorporated herein by reference in their entireties. Such suitable pharmaceutical formulations are most often and preferably formulated to be sterile, isotonic and buffered for stability and comfort. Pharmaceutical compositions for intranasal delivery may also include drops and sprays often prepared to simulate in many respects nasal secretions to ensure maintenance of normal ciliary action. As disclosed in Remington's Pharmaceutical Sciences, 18th Ed., Mack Publishing Co., Easton, PA (1990), which is incorporated herein by reference in its entirety, and well-known to those skilled in the art, suitable formulations are most often and preferably isotonic, slightly buffered to maintain a pH of 5.5 to 6.5, and most often and preferably include antimicrobial preservatives and appropriate drug stabilizers. Pharmaceutical formulations for intraauricular delivery include suspensions and ointments for topical application in the ear. Common solvents for such aural formulations include glycerin and water.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For hydrophobic compounds, a suitable pharmaceutical carrier may be a cosolvent system comprising benzyl alcohol, a nonpolar surfactant, a water-miscible organic polymer, and an aqueous phase. A common cosolvent system used is the VPD co-solvent system, which is a solution of 3% w/v benzyl alcohol, 8% w/v of the nonpolar surfactant Polysorbate 80™, and 65% w/v polyethylene glycol 300, made up to volume in absolute ethanol. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of POLYSORBATE 80™; the fraction size of polyethylene glycol may be varied; other biocompatible polymers may replace polyethylene glycol, e.g., polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

Agents intended to be administered intracellularly may be administered using techniques well known to those of ordinary skill in the art. For example, such agents may be encapsulated into liposomes. All molecules present in an aqueous solution at the time of liposome formation are incorporated into the aqueous interior. The liposomal contents are both protected from the external micro-environment and, because liposomes fuse with cell membranes, are efficiently delivered into the cell cytoplasm. The liposome may be coated with a tissue-specific antibody. The liposomes will be targeted to and taken up selectively by the desired organ. Alternatively, small hydrophobic organic molecules may be directly administered intracellularly.

Additional therapeutic or diagnostic agents may be incorporated into the pharmaceutical compositions. Alternatively or additionally, pharmaceutical compositions may be combined with other compositions that contain other therapeutic or diagnostic agents.

### Methods of Administration

The compounds or pharmaceutical compositions may be administered to the patient by any suitable means. Non-limiting examples of methods of administration include, among others, (a) administration though oral pathways, which administration includes administration in capsule, tablet, granule, spray, syrup, or other such forms; (b) administration through non-oral pathways such as rectal, vaginal, intraurethral, intraocular, intranasal, or intraauricular, which administration includes administration as an aqueous suspension, an oily preparation or the like or as a drip, spray, suppository, salve, ointment or the like; (c) administration via injection, subcutaneously, intraperitoneally, intravenously, intramuscularly, intradermally, intraorbitally, intracapsularly, intraspinally, intrasternally, or the like, including infusion pump delivery; (d) administration locally such as by injection directly in the renal or cardiac area, e.g., by depot implantation: as well as (e) administration topically; as deemed appropriate by those of skill in the art for bringing the compound of the invention into contact with living tissue.

Pharmaceutical compositions suitable for administration include compositions where the active ingredients are contained in an amount effective to achieve its intended purpose. The therapeutically effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

As will be readily apparent to one skilled in the art, the useful in vivo dosage to be administered and the particular mode of administration will vary depending upon the age, weight and mammalian species treated, the particular compounds employed, and the specific use for which these compounds are employed. The determination of effective dosage levels, that is the dosage levels necessary to achieve the desired result, can be accomplished by one skilled in the art using routine pharmacological methods. Typically, human clinical applications of products are commenced at lower dosage levels, with dosage level being increased until the desired effect is achieved. Alternatively, acceptable in vitro studies can be used to establish useful doses and routes of administration of the compositions identified by the present methods using established pharmacological methods.

The exact formulation, route of administration and dosage for the pharmaceutical compositions of the present invention can be chosen by the individual physician in view of the patient's condition. (See e.g., Fingl et al. 1975, in “The Pharmacological Basis of Therapeutics”, which is hereby incorporated herein by reference in its entirety, with particular reference to Ch. 1, p. 1). Typically, the dose range of the composition administered to the patient can be from about 0.5 to 1000 mg/kg of the patient's body weight. The dosage may be a single one or a series of two or more given in the course of one or more days, as is needed by the patient. In instances where human dosages for compounds have been established for at least some condition, the present invention will use those same dosages, or dosages that are between about 0.1% and 500%, more preferably between about 25% and 250% of the established human dosage. Where no human dosage is established, as will be the case for newly-discovered pharmaceutical compounds, a suitable human dosage can be inferred from ED₅₀ or ID₅₀ values, or other appropriate values derived from in vitro or in vivo studies, as qualified by toxicity studies and efficacy studies in animals.

A person of skill in the art would appreciate that an attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

The amount of composition administered may be dependent on the subject being treated, on the subject's weight, the severity of the affliction, the manner of administration and the judgment of the prescribing physician.

Compounds disclosed herein can be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, or monkeys, may be determined using known methods. The efficacy of a particular compound may be established using several recognized methods, such as hi vitro methods, animal models, or human clinical trials. Recognized in vitro models exist for nearly every class of condition, including but not limited to cancer, cardiovascular disease, and various immune dysfunction. Similarly, acceptable animal models may be used to establish efficacy of chemicals to treat such conditions. When selecting a model to determine efficacy, the skilled artisan can be guided by the state of the art to choose an appropriate model, dose, and route of administration, and regime. Of course, human clinical trials can also be used to determine the efficacy of a compound in humans.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound of the invention formulated in a compatible pharmaceutical carrier may also be prepared, placed in an appropriate container, and labeled for treatment of an indicated condition.

### Inflammation

There are many potential causes of inflammation, including physical (burns, frostbite, injury, trauma, radiation, foreign bodies, etc.), biological (infection by pathogens, stress, immune reaction, allergy, associated disease, etc.), chemical (irritants, toxins, alcohols, peels, burns), or psychological stimuli. The hallmark signs of inflammation include heat, pain, redness, swelling, and loss of function. Inflammation is a generic response, and therefore it is considered as a mechanism of innate immunity, as compared to adaptive immunity, which is specific for each pathogen. Too little inflammation could lead to progressive tissue destruction by the harmful stimulus and compromise the survival of the organism. In contrast, chronic inflammation is associated with various diseases, such as hay fever, periodontal disease, atherosclerosis, and osteoarthritis.

Inflammation can be classified as either acute or chronic. Inflammation can also be classified as developmental, meta-inflammation, or inflammaging. Acute inflammation is the initial response of the body to harmful stimuli and is achieved by the increased movement of plasma and leukocytes (especially granulocytes) from the blood into the injured tissues. Acute inflammation occurs immediately upon injury, usually appearing within a few minutes or hours and begins to cease upon the removal of the injurious stimulus. A series of biochemical events propagates and matures the inflammatory response, involving the local vascular system, the immune system, and various cells within the injured tissue. It involves a coordinated and systemic mobilization response locally of various immune, endocrine and neurological mediators of acute inflammation. In a normal healthy response, inflammation becomes activated, pathogen is clear, and the effected cell, tissue, or organ then begins a repair process and inflammation ceases. The process of acute inflammation is initiated by resident immune cells already present in the involved tissue, mainly resident macrophages, dendritic cells, histiocytes, Kupffer cells and mast cells. These cells possess surface receptors known as pattern recognition receptors (PRRs), which recognize (i.e., bind) two subclasses of molecules: pathogen-associated molecular patterns (PAMPs) and damage-associated molecular patterns (DAMPs). PAMPs are compounds that are associated with various pathogens, but which are distinguishable from host molecules. DAMPs are compounds that are associated with host-related injury and cell damage. Cytokines and chemokines promote the migration of neutrophils and macrophages to the site of inflammation. At the onset of an infection, burn, or other injuries, these cells undergo activation (one of the PRRs recognize a PAMP or DAMP) and release inflammatory mediators responsible for the clinical signs of inflammation. Vasodilation and its resulting increased blood flow causes the redness (rubor) and increased heat (calor). Increased permeability of the blood vessels results in an exudation (leakage) of plasma proteins and fluid into the tissue (edema), which manifests itself as swelling (tumor). Some of the released mediators such as bradykinin increase the sensitivity to pain (hyperalgesia, dolor). The mediator molecules also alter the blood vessels to permit the migration of leukocytes, mainly neutrophils and macrophages, to flow out of the blood vessels (extravasation) and into the tissue. The neutrophils migrate along a chemotactic gradient created by the local cells to reach the site of injury. The loss of function (functio laesa) is probably the result of a neurological reflex in response to pain. In addition to cell-derived mediators, several acellular biochemical cascade systems consisting of preformed plasma proteins act in parallel to initiate and propagate the inflammatory response. These include the complement system activated by bacteria and the coagulation and fibrinolysis systems activated by necrosis, e.g. a burn or a trauma. Acute inflammation may be regarded as the first line of defense against injury. Acute inflammatory response requires constant stimulation to be sustained. Inflammatory mediators are short-lived and are quickly degraded in the tissue. Hence, acute inflammation begins to cease once the stimulus has been removed. Pathogens, allergens, toxins, burns, and frostbite are some of the causes of acute inflammation. Toll-like receptors (TLRs) recognize microbial pathogens. Acute inflammation can be a way tissues are protected from injury. Inflammation lasting 2-6 weeks is designated subacute inflammation.

The vascular component of acute inflammation involves the movement of plasma fluid, containing important proteins such as fibrin and immunoglobulins (antibodies), into inflamed tissue. Upon contact with PAMPs, tissue macrophages and mastocytes release vasoactive amines such as histamine and serotonin, as well as eicosanoids such as prostaglandin E2 and leukotriene B4 to remodel the local vasculature. Macrophages and endothelial cells release nitric oxide. These mediators vasodilate and permeabilize the blood vessels, which results in the net distribution of blood plasma from the vessel into the tissue space. The increased collection of fluid into the tissue causes it to swell (edema). This exuded tissue fluid contains various antimicrobial mediators from the plasma such as complement, lysozyme, antibodies, which can immediately deal damage to microbes, and opsonise the microbes in preparation for the cellular phase. If the inflammatory stimulus is a lacerating wound, exuded platelets, coagulants, plasmin and kinins can clot the wounded area and provide haemostasis in the first instance. These clotting mediators also provide a structural staging framework at the inflammatory tissue site in the form of a fibrin lattice—as would construction scaffolding at a construction site—for the purpose of aiding phagocytic debridement and wound repair later on. Some of the exuded tissue fluid is also funnelled by lymphatics to the regional lymph nodes, flushing bacteria along to start the recognition and attack phase of the adaptive immune system. Acute inflammation may be characterized by marked vascular changes, including vasodilation, increased permeability and increased blood flow, which are induced by the actions of various inflammatory mediators. Vasodilation occurs first at the arteriole level, progressing to the capillary level, and brings about a net increase in the amount of blood present, causing the redness and heat of inflammation. Increased permeability of the vessels results in the movement of plasma into the tissues, with resultant stasis due to the increase in the concentration of the cells within blood—a condition characterized by enlarged vessels packed with cells. Stasis allows leukocytes to marginate (move) along the endothelium, a process critical to their recruitment into the tissues. Normal flowing blood prevents this, as the shearing force along the periphery of the vessels moves cells in the blood into the middle of the vessel.

Part of inflammation signaling is the plasma cascade system. This comprises the complement system, the kinin system, the coagulation system, and the fibrinolysis system. The complement system, when activated, creates a cascade of chemical reactions that promotes opsonization, chemotaxis, and agglutination, and produces the MAC. The kinin system generates proteins capable of sustaining vasodilation and other physical inflammatory effects. The coagulation system, or clotting cascade, which forms a protective protein mesh over sites of injury. The fibrinolysis system acts in opposition to the coagulation system to counterbalance clotting and generate several other inflammatory mediators. Also in the plasma cascade system are plasma-driven mediators. Non-limiting examples of plasma-driven mediators include bradykinin, C3, C5a, Factor XII, the membrane attack complex, plasmin, and thrombin.

Prolonged inflammation, known as chronic inflammation, leads to a progressive shift in the type of cells present at the site of inflammation, such as mononuclear cells, and is characterized by simultaneous destruction and healing of the tissue from the inflammatory process. Chronic inflammation may last for months or even years. Macrophages, lymphocytes, and plasma cells predominate in chronic inflammation, in contrast to the neutrophils that predominate in acute inflammation. Diabetes, cardiovascular disease, allergies, and chronic obstructive pulmonary disease (COPD) are examples of diseases mediated by chronic inflammation. Obesity, smoking, stress, and poor diet are some of the factors that promote chronic inflammation. A 2014 study reported that 60% of Americans had at least one chronic inflammatory condition, whereas 42% had more than one.

When inflammation overwhelms the host, systemic inflammatory response syndrome is diagnosed. When it is due to infection, the term sepsis is applied, with the terms bacteremia being applied specifically for bacterial sepsis and viremia specifically to viral sepsis. Vasodilation and organ dysfunction are serious problems associated with widespread infection that may lead to septic shock and death. An infectious organism can escape the confines of the immediate tissue via the circulatory system or lymphatic system, where it may spread to other parts of the body. If an organism is not contained by the actions of acute inflammation it may gain access to the lymphatic system via nearby lymph vessels. An infection of the lymph vessels is known as lymphangitis, and infection of a lymph node is known as lymphadenitis. When lymph nodes cannot destroy all pathogens, the infection spreads further. A pathogen can gain access to the bloodstream through lymphatic drainage into the circulatory system.

Inflammation also induces high systemic levels of acute-phase proteins. In acute inflammation, these proteins prove beneficial; however, in chronic inflammation they can contribute to amyloidosis. These proteins include C-reactive protein, serum amyloid A, and serum amyloid P, which cause a range of systemic effects including fever, increased blood pressure, decreased sweating, malaise, loss of appetite, and somnolence.

The cellular component of inflammation involves leukocytes, which normally reside in blood and must move into the inflamed tissue via extravasation to aid in inflammation. Some act as phagocytes, ingesting bacteria, viruses, and cellular debris. Others release enzymatic granules that damage pathogenic invaders. Leukocytes also release inflammatory mediators that develop and maintain the inflammatory response. In general, acute inflammation is mediated by granulocytes, whereas chronic inflammation is mediated by mononuclear cells such as monocytes and lymphocytes. Various leukocytes, particularly neutrophils, are critically involved in the initiation and maintenance of inflammation. These cells must be able to move to the site of injury from their usual location in the blood, therefore mechanisms exist to recruit and direct leukocytes to the appropriate place. The process of leukocyte movement from the blood to the tissues through the blood vessels is known as extravasation and can be broadly divided up into the steps: (1) leukocyte margination and endothelial adhesion, (2) migration across the endothelium, known as transmigration, via the process of diapedesis, and (3) movement of leukocytes within the tissue via chemotaxis.

Extravasated neutrophils in the cellular phase come into contact with microbes at the inflamed tissue. Phagocytes express cell-surface endocytic pattern recognition receptors (PRRs) that have affinity and efficacy against non-specific microbe-associated molecular patterns (PAMPs). Most PAMPs that bind to endocytic PRRs and initiate phagocytosis are cell wall components, including complex carbohydrates such as mannans and β-glucans, lipopolysaccharides (LPS), peptidoglycans, and surface proteins. Endocytic PRRs on phagocytes reflect these molecular patterns, with C-type lectin receptors binding to mannans and β-glucans, and scavenger receptors binding to LPS. Upon endocytic PRR binding, actin-myosin cytoskeletal rearrangement adjacent to the plasma membrane occurs in a way that endocytoses the plasma membrane containing the PRR-PAMP complex, and the microbe. Phosphatidylinositol and Vps34-Vps15-Beclin1 signalling pathways have been implicated to traffic the endocytosed phagosome to intracellular lysosomes, where fusion of the phagosome and the lysosome produces a phagolysosome. The reactive oxygen species, superoxides and hypochlorite bleach within the phagolysosomes then kill microbes inside the phagocyte. Phagocytic efficacy can be enhanced by opsonization. Plasma derived complement C3b and antibodies that exude into the inflamed tissue during the vascular phase bind to and coat the microbial antigens. As well as endocytic PRRs, phagocytes also express opsonin receptors Fc receptor and complement receptor 1 (CR1), which bind to antibodies and C3b, respectively. The co-stimulation of endocytic PRR and opsonin receptor increases the efficacy of the phagocytic process, enhancing the lysosomal elimination of the infective agent.

There are many important cell-derived mediators for inflammation. While this present application presents data for the compounds of interest inhibiting Lox5, Lox15, Cox2, IFNg, TNFa, IL-6, and IL-1B, it will be understood to those skilled in the art that the compounds may also provide significant direct or indirect inhibition of other key cell-derived mediators. Non-limiting examples of cell-derived mediators include lysosome granules, GM-CSF, histamine, IFNg, IL-6, IL-8, leukotriene B4, LTC4, LTD4, 5-oxo-eicosatetraenoic acid, 5-HETE, prostaglandins, nitric oxide, IL-1, TNFa, and tryptase.

The following description of IL-6 is an excerpt taken from Tanaka and Kishimoto (2012) “Targeting Interleukin-6: All the way to treat autoimmune and inflammatory diseases” Int J Bio Sci. 8(9):1227-1236: “Interleukin-6 (IL-6), initially designated as a B cell differentiation factor, is a representative cytokine featuring redundancy and pleiotropic activity. IL-6 also contributes to host defense against acute environmental stress, while dysregulated persistent IL-6 production has been demonstrated to play a pathological role in various autoimmune and chronic inflammatory diseases. In the early phase of infectious inflammation, IL-6 is produced by monocytes and macrophages immediately after the stimulation of Toll-like receptors (TLRs) with distinct pathogen-associated molecular patterns (PAMPs). In noninfectious inflammations, such as burn or traumatic injury, damage-associated molecular patterns (DAMPs) from damaged or dying cells stimulate TLRs to produce IL-6. This acute IL-6 expression plays a central role in host defense by stimulating various cell populations. When acting on hapatocytes, IL-6 strongly induces a broad spectrum of acute-phase proteins such as C-reactive protein (CRP), serum amyloid A (SAA), fibrinogen, hepcidin, haptoglobin, and antichymotrypsin, whereas it reduces albumin, cytochrome P450, fibronectin, and transferrin. CRP is a good biomarker of inflammation and is used as such in clinical laboratory tests. Its expression mainly depends on IL-6. If the free concentration of the anti-interleukin 6 receptor antibody, tocilizumab is maintained in serum at more than 1 μg/ml, CRP remains negative, so that the serum CRP level is a hallmark for checking whether IL-6 activity is completely blocked in vivo. Continuously high levels of hepcidin induced by IL-6 block iron transporter ferroportin 1 in macrophages, hepatocytes, and gut epithelial cells and lead to hypoferremia and anemia of chronic inflammation, whereas long-term high levels of SAA result in amyloid A amyloidosis. In lymphocytes, IL-6 induces B cell differentiation into immunoglobulin-producing cells. When CD4-positive naïve T cells are primed, a specific cytokine prompts their differentiation into an effector T cell subset. IL-6 together with TGF-β preferentially promotes differentiation of IL-17-producing T helper cells (Th17) that play a crucial role in the induction of autoimmune tissue injury, whereas IL-6 inhibits TGF-β-induced regulatory T cell (Treg) differentiation. The resultant Th17/Treg imbalance leads to breakage of immunological tolerance and is of pathological importance for the development of various autoimmune and chronic inflammatory diseases. IL-6 also induces CD8-positive T cells to generate cytotoxic T cells. The function of IL-6 in hematopoiesis is to induce maturation of megakaryocyte into platelets as well as activation of hematopoietic stem cells. IL-6 production in bone marrow stromal cells generates the receptor activator of NF-kappaB ligand (RANKL), which is an essential factor for the differentiation and activation of osteoclasts and bone resorption, thus leading to osteoporosis. Enhanced angiogenesis and increased vascular permeability are pathological features of inflammation, and these characteristics are due to the excess production of vascular endothelial growth factor (VEGF), which is induced by IL-6 in inflamed lesions such as seen in synovium tissue of rheumatoid arthritis. The promotional activities of IL-6, such as the proliferation of keratinocytes or collagen production in dermal fibroblasts, may contribute to autoimmune skin diseases including psoriasis and systemic sclerosis. Furthermore, IL-6 stimulates the growth of cells such as myeloma/plasmacytoma cells and mesangial cells. IL-6 triggers signal transduction after binding to the IL-6 receptor (IL-6R). There are two forms of IL-6R, a transmembrane 80-kDa form with a short cytoplasmic domain and a soluble form (sIL-6R). After binding of IL-6 to transmembrane IL-6R, the resultant IL-6/IL-6R complex associates with gp130, and the activated IL-6 receptor complex is formed as a hexameric structure consisting of two molecules each of IL-6, IL-6R and gp130 (so-called a classical signaling). The expression of transmembrane IL-6R is limited to a few cell types but the IL-6/sIL-6R complex can also transduce the IL-6 signal to various cells which do not express transmembrane IL-6R but express gp130 (known as a trans-signaling mechanism), so that IL-6 affects a wide variety of cells. When IL-6 is synthesized transiently, it promptly participates in the host defense against environmental stress such as infection and injury and at the same time provides an SOS (warning) signal by triggering a broad spectrum of biological events. Once the source of stress is removed from the host, IL-6-mediated activation of the signal transduction cascade is terminated by negatively regulatory systems in conjunction with the normalization of serum IL-6 and CRP levels. However, dysregulated persistent IL-6 production has been implicated in the development of various autoimmune, chronic inflammatory diseases and even cancers. The reason(s) why such dysregulated continuous IL-6 production is induced remains to be clarified and elucidation of the mechanism(s) underlying persistent IL-6 synthesis in diseases is of particular importance to make their pathogenesis clear. It was found that in human immunodeficiency virus (HIV)-positive cases of multicentric Castleman's diseases all patients were infected with the Kaposi sarcoma-associated herpes virus (KSHV) and that sustained synthesis of both virus-derived IL-6, which directly binds to and stimulates human gp130, and of host-derived human IL-6 contribute to the development of the disease. Moreover, numerous animal models of diseases have also disclosed the pathologic role of IL-6 in disease development and that IL-6 blockade by means of gene-knockout or administration of anti-IL-6 or anti-IL-6R antibody can suppress such disease development either preventively or therapeutically. For example, IL-6 blockade strategy demonstrably limited susceptibility to Castleman's disease-like symptoms in IL-6 transgenic mice, as well as in various mouse models of rheumatoid arthritis, systemic lupus erythematosus, scleroderma, C-peptide-induced myositis, experimental autoimmune uveoretinitis, experimental autoimmune encephalomyelitis, and many other diseases.

Clinical trials of tocilizumab, a humanized anti-IL-6 receptor antibody have verified its efficacy and tolerable safety for patients with rheumatoid arthritis, Castleman's disease and systemic juvenile idiopathic arthritis, resulting in approval of this innovative biologic for treatment of these diseases. Moreover, a considerable number of case reports and pilot studies of off-label use of tocilizumab point to the beneficial effects of tocilizumab for a variety of other phenotypically different autoimmune and chronic inflammatory diseases. Elucidation of the source of IL-6 and of mechanisms through which IL-6 production is dysregulated can thus be expected to lead to clarification of the pathogenesis of various diseases.”

The following description of TNF-α is an excerpt taken from Zhang et al. (2021) “Therapeutic potential of TNFa inhibitors in chronic inflammatory disorders: Past and future” Genes & Dis. 8, 38-47: “Tumor Necrosis Factor a (TNFa) is a small 157-amino-acid cytokine that has a number of functions. Initially TNF was described to have anti-tumor activity, as suggested by its name, and was determined to be in the sera of mice infected with bacillus Calmette-Guerin (BCG) and exposed to endotoxin. Serum with TNF activity in fact demonstrated ability to kill tumor cells in vitro and in vivo. In fact, physician William B. Coley was one of the first to recognize a regression of tumors once a cancer patient contracted a bacterial infection. In 1984, human TNFa cDNA was first cloned after TNFa was purified from HL-60 leukemia cells. The TNFa protein has about 30% homology with human lymphotoxin (also known as TNFb). After that, TNFa was found to be a major regulator of immune function and inflammatory response. In vivo experiments showed that purified mouse TNFa could inhibit the growth or even completely shrink implanted tumors, mostly by inducing a host immune response. Initially, activated macrophages were identified as the major source of TNFa. Later on, many other cell types were also reported to secrete TNFa, including T cells, NK cells, neutrophils, mast cells, and non-immune cells. TNFa plays critical functions in the regulation of the immune system and is fundamental in host protection against microbial infection. An excess of TNFa signaling activity can lead to a number of autoimmune diseases such as rheumatoid arthritis and inflammatory bowel diseases. Inappropriate TNFa activity is also correlated with diseases such as sepsis and cerebral malaria, although it is unclear whether TNFa blockade could lead to effective therapies for these two diseases. There are two forms of TNFa: the soluble ligand form (sTNFa) and the membrane-bound form (tmTNFa). TNFa is expressed as tmTNFa with a transmembrane domain. Trimeric tmTNFa proteins are attached to the cell surface via their transmembrane domains. TNFa releasing enzymes, such as tumor necrosis factor-a converting enzyme (TACE/ADAMI7/CD156q), can cleave tmTNFa and release soluble TNFa into the circulation. TNFa can thus travel to, and affect, target cells that are distant from TNFa-producing cells including macrophages. Structurally TNFa exists as a homotrimer. Accordingly, TNFa receptors (TNFRs) also exist in the form of homotrimers, and are of two types: TNFR1 (p55) and TNFR2 (p75). Mouse TNFa cross-reacts with both human TNFR1 and TNFR2. On the other hand, human TNFa primarily binds mouse TNFR1, and only minimally activates mouse TNFR2. The human sTNFa binds to both TNFR1 and TNFR2 with sub-nanomolar affinity, although its affinity for TNFR1 is slightly higher due to slower dissociation (KD for TNFR1: 0.019 nM, KD for TNFR2: 0.42 nM, at 37_C). While sTNFa activates both TNFR1 and TNFR2, tmTNFa mainly signals through TNFR2 in various systems, such as T cell activation, thymocyte proliferation, and granulocyte/macrophagecolony-stimulating factor (gm-CSF) production. TNFR1 is expressed in most cell types, but is more abundant on white blood cells, including cells of myeloid lineage (e.g., monocytes). TNFR2 expression is limited to hematopoietic cells, as well as some oligodendrocytes and endothelial cells. In a chronic model of proliferative nephritis, expression of TNFR2 in renal endothelial cells was essential for sustained macrophage accumulation. Regulatory T cells, also known as Treg cells, have low TNFR1 but high TNFR2 expression. Treg cells in general function as a negative regulator for the immune system, maintaining tolerance to self-antigens and preventing autoimmune disease. Using TNFR1(_/_) and TNFR2(_/_) mice, Yang et al studied the effect of TNFa on induced Treg (iTreg), and found that TNFR1, although critical for the differentiation of inflammatory T cells such as Th1 and Th17 cells, was not required for the maintenance of iTreg functions. However, TNFR2 was found to be crucial for iTreg differentiation, proliferation, and function. As TNFa may enhance the differentiation and function of iTreg via TNFR2 signaling, TNFR2-agonists or TNFR1-specific antagonists may be effective therapeutics for treating patients with immune diseases.”

“LOX5,” also known as “5LOX,” “5-LOX,” “LOX-5,” and “5-lipoxygenase,” has a complex regulatory mechanism. In general, lipoxygenases are comprised of two domains: N-terminal and C-terminal domains. The N-terminal domain is a regulatory domain and consists mostly of beta-barrels, while the C-terminal domain is a catalytic domain and consists mostly of alpha-helices. The non-heme iron atom is located in the catalytic C-terminal domain, whereas the function of the N-terminal domain is not unambiguously characterized. For 5-LOX, it is clear that the N-terminal domain is essential for translocation to the nuclear membrane whereas for the other LOXs, this is still under debate. Human 5-LOX activity is influenced by the presence of Ca²⁺, which reversibly binds to the enzyme with maximum binding of two Ca²⁺ ions per 5-LOX. Ca²⁺ binding causes an increase in hydrophobicity, which promotes membrane association of 5-LOX. Furthermore, the presence of adenosine triphosphate (ATP) appears to be important for optimal 5-LOX activity.

“15-LOX,”, also known as “LOX15,” “15LOX,” “LOX-15” or “15-lipoxygenase,” may refer to the subtype 15-LOX-1 and/or 15-LOX-2. 15-LOX-1 is highly expressed in leukocytes and airway endothelial cells while, in contrast, 15-LOX-2 is expressed in prostate, lung, cornea, and many tissues such as liver, colon, kidney, spleen, ovary, and brain, but not in leukocytes. Over-expression of lipoxygenases and their pro-inflammatory products, leukotrienes, has been implicated in many human acute and chronic inflammatory diseases such as asthma, atherosclerosis, rheumatoid arthritis, inflammatory bowel diseases, dermatitis, and cancer. In some cases a connection between lipoxygenase activity and activation of the NF-kB pathway has been described.

The following description of IFN-g is an excerpt taken from Lopez de Padilla and Niewold (2016) “The Type I Interferons: Basic Concepts and Clinical Relevance in Immune-mediated Inflammatory Diseases” Gene 576(101), 13-21: “The interferons (IFN)s are a family of cytokines with antiviral, antiproliferative, and antitumor activities, as well as immunomodulatory effects on the innate and adaptive immune responses. Historically, IFNs have been classified into two major types, type I and type II, based on their interactions with the IFN receptor subunits, peptide mapping, and sequencing homology. Recently, a novel class of cytokines with IFN-like activities has been described and designated as type III IFNs. In humans, the type I IFN system consists of a family of IFN proteins encoded by at least 13 LFN alpha (IFNA) subtype genes (IFN-α1, -α2, -α4, -α5, -α6, -α7, -α8, -α10, -α13, -α14, -α16, -α17 and -α21), and one IFN beta gene (IFNB), one IFN-Epsilon gene, one IFN-Kappa gene, and IFN-Omega gene, all of which bind to the type I interferon receptor composed of the IFNAR1 and IFNAR2 chains. Sequence data indicate the human IFNA gene family shares 70-80% sequence homology within the IFNA subtypes, and about 35% identity with IFNB. In humans, the genes encoding IFNA are found as a family of 13 intronless genes clustered together within a region spanning ˜400 kb on the short arm of chromosome 9 (cytogenetic bands 9p22-9p21). There are 12 functional human IFNA gene products. All of these IFN-α proteins exhibit high homology in their primary, secondary, and tertiary structures. They also bind to the same receptor (IFNAR1/IFNAR2) and signal through similar mechanisms eliciting similar biological activity. Currently, there is a small body of experimental data demonstrating differences in the biological activities of human IFN-α subtypes, although some studies suggest that even minor variations in the primary sequences of individual subtypes of human IFNA genes may lead to distinct antiviral and immunoregulatory functions in T cells, B cells, and dendritic cells (DCs). Data from the murine IFNA gene family suggests that diverse IFN-α proteins may vary in their affinity for the IFN receptor subunits, resulting in differences in IFN signaling. Interestingly, mouse fibroblasts transfected with different type I IFNA/B transgenes (i.e., IFNA1, IFNA4, IFNA5, IFNA6, Ifna9 and IFNB) showed different degrees of protection against herpes simplex virus type 1 (HSV-1) and HSV-2; suggesting differences in the downstream activation of genes responsible for the antiviral activities of IFN-α subtypes. Some studies also suggest differences in cell- and ligand-specific expression and different kinetics between IFN-α subtypes, suggesting other mechanisms for diversity in downstream response beyond conformational changes at the type I IFN receptor. The high degree of amino acid sequence similarity within the IFN-α proteins suggests a common ancestral gene. Gene clusters such as the IFNA cluster are genomic regions that comprise multiple similar copies in close proximity and are thought to be generated by local duplication of a common ancestral segment. A study published by Woelk and colleagues using gene conversion analysis of 156 IFNA genes from mammalian species (chimpanzee, dog, mouse, rat, and rhesus macaque) in which gene-specific clustering is also evident, identified specific sequences and fragments involved in gene conversion and gene duplication events. This study suggested that both of these evolutionary mechanisms contributed to the evolution of IFNA gene clusters. Other studies have been unable to clarify whether gene conversion or recent duplication play a role for gene-specific clustering of IFNA genes. An evolutionary analysis of human and mouse IFNA genes failed to find evidence of gene conversion in humans but some interlocus recombination was identified among mouse IFNA genes. Type I IFNs elicit antiviral, antiproliferative and immunomodulatory responses by binding to the type I interferon receptor. The receptor consists of the IFNAR1 and IFNAR2 transmembrane proteins, and two associated cytoplasmic tyrosine kinases, the Janus kinase 1 (JAKI) and tyrosine kinase 2 (TYK2). The IFNAR2 subunit is considered as the primary binding chain as it binds type I IFNs with relatively high affinity, whereas the IFNAR1 subunit does not bind type I IFNs with detectable affinity but is absolutely required for signal transduction from the heterodimeric IFNAR complex and for type I IFN biological activity. Thus, both the IFNAR1 and IFNAR2 subunits are required to mediate the biological effects of all type I IFNs. The biological effects of IFNs are mediated through the Janus kinase/signal transducer and activator of transcription (JAK/STAT) pathway. STAT1 and STAT2 mediate the antiviral and inflammatory effects of IFN-α/IFN-β. Upon IFNAR engagement, IFNs induce tyrosine phosphorylation of STAT1 and STAT2 proteins and, together with IFN-regulatory factor 9 (IRF9), form the IFN-stimulated gene factor 3 (ISGF3) transcription factor complex, which then translocates to the nucleus and binds to IFN-stimulated response elements (ISREs) in the promoters of IFN-regulated genes (IRGs). In addition, canonical type I IFN signaling may activate STAT1 homodimers that bind to interferon-gamma-activating factor (GAF), which translocates to the nucleus and activates transcription of IFN-stimulated genes. In contrast, IFN-α-activated STAT3 is thought to inhibit STAT1-dependent gene activation, thereby down-regulating IFN-α-mediated induction of inflammatory mediators, attenuating the inflammatory properties of type I IFNs. The canonical IFN-JAK/STAT signal transduction pathway is not isolated, but communicates extensively with other signal transduction pathways such as the innate pattern recognition receptors (PRRs), which include Toll-like receptors (TLRs), RIG-I-like receptors (RLGs), NOD-like receptors, and C-type lectin receptors. The virus-induced expression of IFNA/IFNB genes is primarily controlled at the gene transcription level by the IRFs and IFN-stimulated genes. Immune complexes (ICs) containing nucleic acids can access intracellular TLRs (TLR3, TLR7/8 and TLR9) after binding to Fc receptors and induce IFN-α production by IRF3, IRF7, and IRF5. Signaling through TLRs can broadly be categorized into two pathways; the MyD88 and the TRIF-dependent pathway. All TLRs, except TLR3, activate through the MyD88-dependent pathway. Only TLR3 and TLR4 activate through the TRIF-dependent pathway. The MyD88-dependent pathway recruits several effector molecules such as IRAK1/4 and tumor necrosis factor receptor-associated factor 6 (TRAF6). These molecules are linked to at least three major downstream pathways: the NF-κB pathway, the pathway involving mitogen-activated protein kinases (MAPKs), and IRF pathways. Depending on the stimulus and the responding cell types, activation of these pathways results in transcription of various cytokines including IFN-α/P. In human plasmacytoid DCs (pDCs), IRFs such as IRF3, 5, and 7 are activated by TLR7 and TLR9 signaling pathways, enabling type I IFN production. In a model of virus-mediated IFNA/IFNB gene induction in fibroblasts, IRF3 and IRF7 were both required for efficient induction of IFNA and IFNB genes, as they cooperate with each other as DNA-binding transcription factors at the promoter. Studies have suggested that IRF3 is mainly responsible for the initial induction of IFNB, whereas IRF7 is involved in the late phase of both IFNA and IFNB gene induction. Honda et al. showed a robust induction of IFNA/IFNB mRNA expression upon CpGstimulation (a TLR9 agonist) of splenic-derived pDCs, which was abolished in splenic-derived pDCs from Irf7−/−mice, despite the normal expression of TLR9 mRNA. In contrast, induction of IFNA/IFNB mRNA expression occurred normally in Irf3−/−pDCs. Similar results were obtained upon stimulation with synthetic single-stranded RNA (TLR7 agonist). Their results suggest that IRF7 is essential and IRF3 is dispensable for MyD88-dependent induction of IFNA/IFNB genes via the TLR9 and TLR7 in pDCs in these animal models. The induction of IFNA gene expression may represent a finely tuned mechanism by which different cell types within the innate and adaptive immunity systems produce specific IFN-α subtypes in response to different stimuli, and in different physiological and pathological conditions. Although IFN-α and IFN-β are produced by a wide range of cells such as macrophages, fibroblasts, and endothelial cells, plasmacytoid dendritic cells (pDCs) are thought to be the major cell type responsible for producing high levels of IFN-α in response to RNA or DNA viruses. PDCs are thought to produce type I IFN in response to nucleic acid-containing immune complexes through activation of TLRs 7 and 9, which is relevant in autoimmune conditions such as SLE in which these types of immune complexes are prevalent. IFN-α has been reported to modulate the number and function of several key immune effector cells such as B cells, T effector cells, and regulatory T cells in autoimmune disease. For instance, type I IFN induced by pDCs significantly stimulated full differentiation of autoreactive B cells into Ig-secreting plasma cells and promote B cell survival in B cells purified from anti-snRNP Ig Tg mice upon TLR7/9 stimulation. These responses were partially abrogated by neutralization of IFN-α/β and IL-6. IFN-α also plays a major role in T cells by inducing immunogenic T cell responses. Ag-specific naïve CD8+ T cells primed in the presence of IFN-α undergo marked proliferation and acquisition of effector functions. In addition, T cells isolated from the skin of psoriasis patients show an increased and prolonged IFN-α signaling pathway activation when compared with infiltrating T cells from skin of non-psoriatic donors. With regard to Foxp3+ Treg cells, it has been shown that IFN-α mediates the inactivation of human Treg cells by downregulating intracellular cAMP levels and negative regulation of T-cell receptor signaling, and might be responsible for autoimmune dysfunctions associated with IFN-α treatment of hematologic malignancies. Similarly, the blockade of Treg cell development by IFN-α-producing antigen-presenting cells has been suggested as a pathogenic factor in untreated active SLE patients.”

Inflammatory abnormalities are a large group of disorders that underlie a vast variety of human diseases. The immune system is often involved with inflammatory disorders, demonstrated in both allergic reactions and some myopathies, with many immune system disorders resulting in abnormal inflammation. Non-immune diseases with causal origins in inflammatory processes include cancer, atherosclerosis, and ischemic heart disease. Non-limiting examples of disorders associated with inflammation include: acne vulgaris, allergies, asthma, atherosclerosis, autoimmune diseases, autoinflammatory diseases, cancer, celiac disease, chronic prostatitis, colitis, depression, diverticulitis, familial Mediterranean fever, glomerulonephritis, hidradenitis suppurativa, HIV infection/AIDS, hypersensitivities, inflammatory bowel disease, interstitial cystitis, leukocyte defects, lichen planus, mast cell activation syndrome, mastocytosis, myopathies, otitis, pelvic inflammatory diseases, peripheral ulcerative keratitis, pneumonia, reperfusion injury, rheumatic fever, rheumatic arthritis, rhinitis, sarcoidosis, transplant rejection, vasculitis, and vitamin A deficiency.

With the discovery of interleukins (IL), the concept of systemic inflammation gained acceptance. Although the processes involved are identical to tissue inflammation, systemic inflammation is not confined to a particular tissue but involves the endothelium and other organ systems. Chronic inflammation is widely observed in obesity. Obese people commonly have many elevated markers of inflammation, including: IL-6, IL-8, IL-18, TNF-a, CRP, insulin, blood glucose, and leptin. Low-grade chronic inflammation is characterized by a two- to threefold increase in the systemic concentrations of cytokines such as TNF-α, IL-6, and CRP. Waist circumference correlates significantly with systemic inflammatory response. Loss of white adipose tissue reduces levels of inflammation markers. The association of systemic inflammation with insulin resistance and type II diabetes, and with atherosclerosis is under preliminary research, although rigorous clinical trials have not been conducted to confirm such relationships. C-reactive protein (CRP) is generated at a higher level in obese people and may increase the risk for cardiovascular diseases.

There is evidence linking inflammation and depression. Inflammatory processes can be triggered by negative cognitions or their consequences, such as stress, violence, or deprivation. In addition, there is increasing evidence that inflammation can cause depression because of the increase of cytokines, setting the brain into a “sickness mode”. Classical symptoms of being physically sick like lethargy show a large overlap in behaviors that characterize depression. Levels of cytokines tend to increase sharply during the depressive episodes of people with bipolar disorder and drop off during remission. Furthermore, it has been shown in clinical trials that anti-inflammatory medicines taken in addition to antidepressants not only significantly improves symptoms but also increases the proportion of subjects positively responding to treatment. Inflammations that lead to serious depression could be caused by common infections such as those caused by a virus, bacteria or even parasites.

Specific patterns of inflammation are seen during particular situations that arise in the body, such as when inflammation occurs on an epithelial surface, or pyogenic bacteria are involved. Non-limiting forms of inflammation include granulomatous inflammation, fibrinous inflammation, purulent inflammation, serous inflammation, and ulcerative inflammation.

Inflammation has also been classified as Type 1 and Type 2 based on the type of cytokines and helper T cells (Th1 and Th2) involved. It will be understood to one skilled in the art that the compounds disclosed herein may have therapeutic properties against both Type 1 and Type 2 inflammation.

In some embodiments, the compounds disclosed herein inhibit TNF activity with an EC50 that is, is about, is at most, or is at most about 50 μM, 30 μM, 20 μM, 19 μM, 18.5 μM, 15 μM, 10 μM, 9 μM, 8.7 μM, 8.5 μM, 6 μM, 5 μM, 3 μM, 2 μM, and/or 1 μM, or any range of values therebetween, for example such as between 6 and 19 μM.

In some embodiments, the compounds disclosed herein inhibit IL-6 activity with an EC50 that is, is about, is at most, or is at most about 50 μM, 30 μM, 20 μM, 18 μM, 10 μM, 5 μM, and/or at most about 1 μM, or any range of values therebetween, for example such as between 10 and 20 μM.

In some embodiments, the compounds disclosed herein inhibit IFN-gamma activity with an EC50 that is, is about, is at most, or is at most about 10 PM, 5 μM, 4 μM, 3 μM, 2.5 μM, 2 μM, 1.9 μM, 1.8 μM, 1.1 μM, 1 μM, 0.9 μM, 0.5 μM, and/or 0.1 μM, or any range of values therebetween, for example such as between 1 and 5 μM.

In some embodiments, the compounds disclosed herein inhibit IL-1β activity with an EC50 that is, is about, is at most, or is at most about 50 μM, 30 PM, 20 μM, 10 μM, 5 μM, 1 μM, 0.9 μM, 0.1 μM, and/or 0.01 μM, or any rage of values therebetween, for example such as between 0.1 and 20 μM.

In some embodiments, the compounds disclosed herein inhibit Lox-5 activity with an EC50 that is, is about, is at most, or is at most about 10 μM, 1 μM, 0.6 μM, 0.5 μM, 0.4 μM, 0.3 μM, 0.2 μM, 0.1 μM, 0.05 μM, and/or 0.01 μM, or any range of values therebetween, for example such as between 0.1 and 0.6 μM.

In some embodiments, the compounds disclosed herein inhibit Lox-15 activity with an EC50 that is, is about, is at most, or is at most 50 μM, 30 μM, 20 μM, 18 μM, 10 μM, 8 μM, 5 μM, 3 μM, 2.8 μM, 2 μM, 1 μM, 0.9 μM, 0.5 μM, and/or 0.1 μM, or any range of values therebetween, for example such as between 0.9 and 30 μM.

In some embodiments, the compounds disclosed herein inhibit IL-10 activity with an EC50 that is, is about, is at most, or is at most about 30 μM, 20 μM, 10 μM, 5 μM, 2 μM, 1.7 μM, 1.5 μM, 1.2 μM, 1.1 μM, 1 μM, 0.5 μM, and/or 0.1 μM, or any range of values therebetween, for example such as between 1 and 20 μM.

In some embodiments, the compounds disclosed herein inhibit the CB2 receptor. In some embodiments the compound inhibits CB2 receptor activity with an IC50 that is, is about, is at most, or is at most about 0.06 μM, 0.07 μM, 0.08 μM, 0.09 μM, 0.1 μM, 0.15 μM, 0.2 μM, 0.4 μM, 0.6 μM, 1 μM, and/or 2 μM, or any range of values therebetween, for example such as between 0.06 and 2 μM.

In some embodiments, the compounds disclosed herein selectively inhibit the CB2 receptor over the CB1 receptor. In some embodiments, the selectivity for CB2 receptor inhibition over the CB1 receptor inhibition (calculated as the fold-difference in IC50 values), is, is about, is at least, or is at least about 2-fold, 5-fold, 10-fold, 25-fold, 50-fold, 75-fold, and/or 100-fold, or any range of values therebetween, for example such as between 2-fold and 25-fold.

In some embodiments, the compounds disclosed herein are well tolerated by the mammalian and/or human cells. In some embodiments, the cells are normal, healthy cells. In some embodiments, the cells are cancer or tumor cells. In some embodiments, the cells are immune cells. In some embodiments, the cells are at least one of epithelial cells, stromal cells, white blood cells, and/or melanocyte cells. In some embodiments, the cells are at least one of epithelioid carcinoma cells, pancreatic carcinoma cells, prostatic adenocarcinoma cells, myeloid leukemia cells, and/or melanoma cells. In some embodiments, the cells are at least one of PANC-1, PC3, HL-60, and/or SK-MEL-28 cells. In some embodiments, cells maintain an, an about, an at least, or an at least about 50%, 70%, 80%, 85%, 90%, 95%, 99%, and/or 100%/o viability, or any range of values therebetween, for example such as 80% and 100% viability, when contacted with 30 μM of compound. In some embodiments, the compounds inhibit cell viability with an EC50 that is at least about 30 μM. In some embodiments, the compounds have, have about, have at least, or have at least about 100-fold, 200-fold, 250-fold, 400-fold, 600-fold, 800-fold, 1,000-fold, 10,000-fold, and/or 15,0000-fold, or any range of values therebetween, for example such as between 250 and 15,000-fold, decreased toxicity to a cell compared to an equivalent dosage of staurosporine

Some embodiments provide a method of treating, ameliorating, or preventing the recurrence of an inflammatory condition selected from the group consisting of acute inflammation, chronic inflammation, developmental inflammation, meta-inflammation and inflammaging in a subject having an inflammatory condition. Some embodiments provide a use of a compound or composition in the preparation of a medicament for treating, ameliorating, or preventing the recurrence of an inflammatory condition selected from the group consisting of acute inflammation, chronic inflammation, developmental inflammation, meta-inflammation and inflammaging in a subject having an inflammatory condition. In some embodiments, the method and/or use comprises administering an effective dose of a composition comprising (i) at least one of a pharmaceutically effective carrier, a pharmaceutically effective diluent, and a pharmaceutically effective excipient, and (ii) a compound of formula (I). In some embodiments, R^{A} and R^{B} of formula (I) are each selected from the group consisting of: —H, —F, —Cl, —Br, —I, —CH₃, —CH₂CH₃, —CF₃, —OH, —OCH₃, —OCH₂CH₃, —OCONH₂, —O(CO)CH₃, —O(CO)CH₂CH₃, —CN, —NH₂, —NHCH₂CH₂CH₃, —NHCH₂CH₃, —N(CH₃)₂ and —NHCH(CH₃)₂. In some embodiments, each of X and Y of formula (I) are independently selected from the group consisting of: —OH, ═O, —OCH₃, —OCOCH₃ and —OCOCH₂CH₃. In some embodiments, R¹ of formula (I) is a moiety of formula (II). In some embodiments, each dashed line of R¹ of formula (I) is independently selected from representing a carbon-carbon single bond and representing a carbon-carbon double bond. In some embodiments, R² of formula (I) is either a moiety of the formula (III-A), —(CH₂)ₙCH₃, wherein n in an integer greater than one and less than seven, or a moiety of the formula (III-B), —(CH₂)ₘ(CH═CH)(CH₂)ₚCH₃, wherein m is an integer greater than zero and less than four, and wherein p is an integer greater than or equal to zero and less than four, and wherein the sum of m and p is not greater than four. In some embodiments, the compound is not any of the compounds of formulas (IV, VI and VIII). In some embodiments, the chronic inflammation results from at least one of anemia, arthritis, asthma, autoimmune disease, bone disease, bowel disease, cancer, cardiovascular disease, celiac disease, cerebrovascular disease, Crone's disease, diabetes, dysglycemia, eczema, fibromyalgia, gastrointestinal disorder, gingivitis, granulomatosis, Grave's disease, Hashimoto's disease, hemolytic anemia, inflammatory bowel disease, joint disease, leukemia, lupus, metabolic disease, muscular dystrophy, neuropathy, obesity, ocular disease, periodontitis, psoriasis, pulmonary disease, renal disease, rheumatoid arthritis, scleroderma, sclerosis, skin disease, thyroid disease, thyroiditis, ulceratic colitis, vitiligo, Wegener's disease, Castleman's disease, pulmonary arterial hypentension, atopic dermititus, and sciatica. In some embodiments, the chronic inflammation is associated with at least one of anemia, arthritis, asthma, autoimmune disease, bone disease, bowel disease, cancer, cardiovascular disease, celiac disease, cerebrovascular disease, Crone's disease, diabetes, dysglycemia, eczema, fibromyalgia, gastrointestinal disorder, gingivitis, granulomatosis, Grave's disease, Hashimoto's disease, hemolytic anemia, inflammatory bowel disease, joint disease, leukemia, lupus, metabolic disease, muscular dystrophy, neuropathy, obesity, ocular disease, periodontitis, psoriasis, pulmonary disease, renal disease, rheumatoid arthritis, scleroderma, sclerosis, skin disease, thyroid disease, thyroiditis, ulceratic colitis, vitiligo, Wegener's disease, Castleman's disease, pulmonary arterial hypentension, atopic dermititus, and sciatica. In some embodiments, the inflammation is not neuroinflammation. In some embodiments, the therapeutic effect of the compound is not attributed to its interaction with a one or more PPAR-γ receptor in the subject.

Some embodiments disclosed herein provide a method of treating, ameliorating, reducing, or preventing the recurrence of a one or more symptom of a disease or disorder in a subject in need thereof. Some embodiments disclosed herein provide a use of a compound or composition in the preparation of a medicament for treating, ameliorating, reducing, or preventing the recurrence of a one or more symptom of a disease or disorder in a subject in need thereof. In some embodiments, the method and/or use comprises administering a compound of formula (I). In some embodiments, each of R^{A} and R^{B} of formula (I) is independently selected from the group consisting of: —H, —F, —Cl, —Br, —I, —CH₃, —CH₂CH₃, —CF₃, —OH, —OCH₃, —OCH₂CH₃, —O(CO)NH₂, —O(CO)CH₃, —O(CO)CH₂CH₃, —CN, —NH₂, —NHCH₂CH₂CH₃, —NHCH₂CH₃, —N(CH₃)₂ and —NHCH(CH₃)₂. In some embodiments, each of X and Y of formula (I) is independently selected from the group consisting of: —OH, ═O, —OCH₃, —O(CO)CH₃ and —O(CO)CH₂CH₃. In some embodiments, R¹ of formula (I) is a moiety of formula (II). In some embodiments, each dashed bond of R¹ of formula (I) is individually selected from representing a carbon-carbon single bond and representing a carbon-carbon double bond. In some embodiments, R² of formula (I) is a moiety of formula (I). In some embodiments, the dashed bond of R² of formula (I) is selected from representing a carbon-carbon single bond and representing a carbon-carbon double bond. In some embodiments, the disease or disorder is associated with the activity of or the expression of one or more of TNF, IL-6, IL-1p, IL-10, IFNg, Lox5, CB2, and/or Lox15. In some embodiments, R^{B} is —OH. In some embodiments, R^{B} is selected from the group consisting of —OCH₃, —OCH₂CH₃ and —NH₂. In some embodiments, R^{A} is —H. In some embodiments, R^{A} is selected from the group consisting of —F, —Cl, and —Br. In some embodiments, each of X and Y is ═O. In some embodiments, each of X and Y is independently selected from the group consisting of —OCOCH₃ and —O(CO)CH₂CH₃. In some embodiments, the compound is selected from compounds IV-VIII. In some embodiments, the compound is compound IV. In some embodiments, the compound is compound V. In some embodiments, the compound is compound VI. In some embodiments, the compound is compound VII. In some embodiments, the compound is compound VIII. In some embodiments, the subject is mammalian or human. In some embodiments, the one or more symptom is selected from the group consisting of systemic inflammation, acute inflammation, chronic inflammation, developmental inflammation, meta-inflammation, and inflammaging. In some embodiments, the method of treatment and/or use results in the amelioration, reduction, or prevention of inflammation in the subject. In some embodiments, the compound is formulated with at least one of a pharmaceutically effective carrier, a pharmaceutically effective diluent, and a pharmaceutically effective excipient. In some embodiments, the compound is formulated for intradermal, topical, transdermal, oral, buccal, sublingual, nasal, or intravenous application.

In some embodiments, the disease or disorder is associated with the activity and/or expression of one or more of TNF, IL-6, IL-10, IL-1β, IFNg, Lox-5 (also called 5Lox), Lox-15 (also called 15Lox), and CB2. In some embodiments, the disease or disorder is associated with the activity and/or expression of TNF. In some embodiments, the compound inhibits TNF activity with an EC50 that is at most about 20 μM. In some embodiments, the disease or disorder is selected from the group consisting of acute myeloid leukemia, acute respiratory distress, amyloidosis, ankylosing spondylitis, arthritis, autoimmune disease, axial spondyloarthritis, back pain, Behcet's syndrome, burn-associated inflammation, cancer, cardiovascular disease, cerebral malaria, chronic lymphocytic leukemia. Crohn's disease, diabetes, Dupuytren's disease, fibrosis, fingernail psoriasis, fracture, Grave's disease, hidradenitis suppurativa, HIV infection, immune-mediated inflammatory disease, infectious disease, inflammatory bowel disease, influenza, injury, joint pain, lupus, multiple sclerosis, neck pain, non-Infectious Intermediate, Posterior, and Panuveitis, obesity, onchocerciasis, organ injury, osteoarthritis, pediatric Crohn's disease, pediatric plaque psoriasis, pediatric ulcerative colitis, plaque psoriasis, polyarticular juvenile idiopathic arthritis, post-operative cognitive dysfunction, psoriatic arthritis, rheumatoid arthritis, sepsis, spondyloarthritis, systemic lupus erythematosus nephritis, tissue damage, trypanosomiasis, type I diabetes, ulcerative colitis, uveitis, and ventilator-induced lung injury. In some embodiments, the disease or disorder is associated with the activity and/or expression of IL-6. In some embodiments, the compound inhibits IL-6 activity with an EC50 that is at most about 20 μM. In some embodiments, the disease or disorder is selected from the group consisting of acquired hemophilia A, adult onset Still's disease, amyloid A amyloidosis, ankylosing spondylitis, autoimmune disease, autoimmune hemolytic anemia, atopic dermatitis, Behcet's disease, burn-associated inflammation, cancer, cardiovascular disease, cardiovascular disease in rheumatoid arthritis, Castleman's disease, chronic glomerulonephritis, colorectal cancer, Crohn's disease, cryoglobulinemia, diabetes, giant cell arteritis, acute graft-versus-host disease, graft-versus-host disease, Grave's disease, Grave's ophthalmopathy, hepatitis B infection, HIV infection, HTLV-1 infection, infectious inflammation, injury-associated inflammation, KSHV infection, lupus, lupus erythematosus, myeloperoxidase-antineutrophil cytoplasmic antibody-associated crescentic glomerulonephritis, neuromyelitis optica, non-infectious inflammation, obesity, non-ST-elevation myocardial infarction, organ rejection, organ transplant rejection, polychondritis, polymyalgia rheumatica, polymyositis, pulmonary arterial hypertension, relapsing polychondritis, remitting seronegative symmetrical synovitis with pitting edema, rheumatoid arthritis, rheumatoid vasculitis, sciatica, sclerosis, spondyloarthritis, systemic juvenile idiopathic arthritis, systemic lupus erythematosus, systemic sclerosis, Takayasu arteritis, tumor necrosis factor receptor-associated periodic syndrome, type II diabetes, uveitis, and vasculitis syndrome. In some embodiments, the disease or disorder is associated with the activity and/or expression of IL-1β. In some embodiments, the compound inhibits IL-1β activity with an EC50 that is at most about 10 μM. In some embodiments, the disease or disorder is selected from the group consisting of acne, acute respiratory distress syndrome, amyloidosis, antisynthetase syndrome, arthritis, atherosclerosis, autoinflammation, autoimmune disease, Behcet's disease, Blau syndrome, bone disease, cancer, cardiovascular disease, chondrocalcinosis, chronic inflammatory neurological cutaneous articular syndrome, chronic obstructive pulmonary disease, Crohn's disease, cryopyrin-associated periodic syndrome, deficiency in IL-1 receptor antagonist, diabetes, disseminated intravascular coagulation, Erdheim-Chester syndrome, familial cold-induced autoinflammatory syndrome, familial Mediterranean fever, gout, graft-versus-host disease, headache, heart failure, hyper IgD syndrome, hyperostosis, hypoglycemia, inanition, infectious disease, inflammasome-associated disease, injury, interstitial lung disease, intestine inflammation, inflammasome-associated disease, irritable bowel syndrome, ischemic disease, joint disease, macrophage activation syndrome, macular degeneration, Majeed syndrome, malignancy, metabolic syndrome disorder, mevalonate kinase deficiency syndrome, microbial infection, Muckle-Wells syndrome, multiple sclerosis, myeloma, myocardial infarction, non-cancer inflammatory disease, obesity, ocular disease, osteitis, osteoarthritis, pericarditis, PFAPA syndrome, post myocardial infarction heart failure, psoriasis, pulmonary disease, pustulosis, pyoderma gangrenosum, pyogenic arthritis, pyogenic arthritis-pyoderma gangrenosum-acne syndrome, recurrent idiopathic pericarditis, recurrent pericarditis, redness at injection site, relapsing chondritis, renal dysfunction, retinal degeneration, rheumatoid arthritis, Schnitzler syndrome, sepsis, septic shock syndrome, sclerosis, sinus inflammation, Sjögren syndrome, smoldering multiple myeloma, Still's disease, Sweet syndrome, synovitis, systemic lupus erythematosus, systemic-onset juvenile idiopathic arthritis, TNF receptor-associated periodic syndrome, type I diabetes, type II diabetes, upper respiratory tract inflammation, urticarial vasculitis, and uveitis. In some embodiments, the disease or disorder is associated with the activity and/or expression of IFNg. In some embodiments, the compound inhibits IFNg activity with an EC50 that is at most about 1 μM. In some embodiments, the disease or disorder is selected from the group consisting of anti-TNF-induced lupus, arthritis, autoimmune diabetes, autoimmune disease, autoimmune encephalomyelitis, autoimmune myositis, Behcet's disease, cutaneous autoimmune disorder, dermatomyositis, diabetes, infectious disease, inflammatory bowel disease, injury, juvenile idiopathic arthritis, leukocytoclastic vasculitis, lupus, metabolic immune disorder, multiple sclerosis, obesity, psoriatic arthritis, psoriasis, psoriasiform eruptions, rheumatoid arthritis, sclerosis, Sjogren's syndrome, synovial inflammation, systemic lupus erythematosus, systemic sclerosis, thrombosis, type I diabetes, and vasculitis. In some embodiments, the disease or disorder is associated with the activity and/or expression of Lox5. In some embodiments, the compound inhibits Lox5 activity with an EC50 that is at most about 0.3 μM. In some embodiments, the disease or disorder is selected from the group consisting of an allergic reaction, allergy-associated inflammation, angioedema, arthritis, asthma, atherosclerosis, autoimmune disorder, burn-associated inflammation, cardiovascular disease, cancer, chronic obstructive pulmonary disease, conjunctivitis, diabetes, eczema, H. pylori infection, infectious disease, inflammatory bowel disease, injury, NSAID hypersensitivity reaction, NSAID-induced non-allergic reaction, obesity, psoriasis, rheumatoid arthritis, rhinitis, and urticaria. In some embodiments, the disease or disorder is associated with the activity and/or expression of Lox15. In some embodiments, the compound inhibits Lox15 activity with an EC50 that is at most about 1 μM. In some embodiments, the disease or disorder is selected from the group consisting of an allergic reaction, allergy-associated inflammation, angioedema, arthritis, asthma, atherosclerosis, autoimmune disorder, burn-associated inflammation, cardiovascular disease, cancer, chronic obstructive pulmonary disease, conjunctivitis, diabetes, eczema, H. pylori infection, infectious disease, inflammatory bowel disease, injury, NSAID hypersensitivity reaction, NSAID-induced non-allergic reaction, obesity, psoriasis, rheumatoid arthritis, rhinitis, and urticaria.

In some embodiments, the disease or disorder is associated with the activity and/or expression of IL-10. In some embodiments, the compound inhibits IL-10 activity with an EC50 that is at most about 1 μM, or alternatively at most about 1.1 μM. In some embodiments, the disease or disorder is selected from the group consisting of allergic reaction, allergy-associated immune response, arthritis, psoriasis, diffuse large B-cell lymphoma, cervical cancer, oral cancer, tuberculosis, Crohn's disease, asthma, rheumatoid arthritis, systemic lupus erythematosus, general inflammation, inflammatory bowel disease, cancer, tuberculosis, infectious disease, allergies, non-small cell lung cancer, diffuse large B-cell lymphoma, colon cancer, and prostate cancer. In some embodiments, the disease or disorder is associated with the activity and/or expression of CB2. In some embodiments, the compound inhibits CB2 activity with an EC50 that is at most about 0.12 μM. In some embodiments, the disease or disorder is selected from the group consisting of allergic reaction, allergy-associated inflammation, Alzheimer's Disease, angioedema, arthritis, asthma, atherosclerosis, autoimmune disorder, burn-associated inflammation, cardiovascular disease, cancer, chronic obstructive pulmonary disease, conjunctivitis, diabetes, eczema, erythematosus, general inflammation, H. pylori infection, infectious disease, inflammatory disease, inflammatory bowel disease, injury, neoplasms, neurodegenerative disease, NSAID hypersensitivity reaction, NSAID-induced non-allergic reaction, obesity, Parkinson's Disease, psoriasis, systemic lupus rheumatoid arthritis, rhinitis, tuberculosis, and urticaria.

### Examples

### General Procedures

Additional embodiments are disclosed in further detail in the following examples, which are not in any way intended to limit the scope of the claims.

Materials used in preparing compounds of Formula I-VIII, described herein may be made by known methods or are commercially available. In these reactions, it is also possible to make use of variants which are themselves known to those of ordinary skill in this art, but are not mentioned in greater detail. The skilled artisan given the literature and this disclosure is well equipped to prepare any of the compounds.

It is recognized that the skilled artisan in the art of organic chemistry can readily carry out manipulations without further direction, that is, it is well within the scope and practice of the skilled artisan to carry out these manipulations. These include reduction of carbonyl compounds to their corresponding alcohols, oxidations, acylations, aromatic substitutions, both electrophilic and nucleophilic, etherifications, esterification and saponification and the like. These manipulations are discussed in standard texts such as March's Advanced Organic Chemistry (Wiley), Carey and Sundberg, Advanced Organic Chemistry (incorporated herein by reference in their entirety) and the like.

The skilled artisan will readily appreciate that certain reactions are best carried out when other functionality is masked or protected in the molecule, thus avoiding any undesirable side reactions and/or increasing the yield of the reaction. Often the skilled artisan utilizes protecting groups to accomplish such increased yields or to avoid the undesired reactions. These reactions are found in the literature and are also well within the scope of the skilled artisan.

The following example schemes are provided for the guidance of the reader, and represent preferred methods for making the compounds exemplified herein. These methods are not limiting, and it will be apparent that other routes may be employed to prepare these compounds. Such methods specifically include solid phase based chemistries, including combinatorial chemistry. The skilled artisan is thoroughly equipped to prepare these compounds by those methods given the literature and this disclosure. The compound numberings used in the synthetic schemes depicted below are meant for those specific schemes only, and should not be construed as or confused with same numberings in other sections of the application.

Trademarks used herein are examples only and reflect illustrative materials used at the time of the invention. The skilled artisan will recognize that variations in lot, manufacturing processes, and the like, are expected. Hence the examples, and the trademarks used in them are non-limiting, and they are not intended to be limiting, but are merely an illustration of how a skilled artisan may choose to perform one or more of the embodiments of the invention.

The following example schemes are provided for the guidance of the reader, and collectively represent an example method for making the compounds provided herein. Furthermore, other methods for preparing compounds described herein will be readily apparent to the person of ordinary skill in the art in light of the following reaction schemes and examples. Unless otherwise indicated, all variables are as defined above.

Although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the invention.

### Example 1: Preparation of Cannabigerol Quinone (Formula (VI))

Olivetol (500 mg, 2.77 mmol) and geraniol (285 mg, 1.85 mmol) was dissolved in dichloroethane (10 mL) whereupon acidic alumina (3.7 g) was added. The mixture was heated to reflux and stirred for 5 hours. When the reaction had gone to completion as monitored by LCMS, it was cooled and filtered through a Celite plug. The solvent was then concentrated in vacuo to afford a yellow oil. The residue was purified via flash chromatography eluted with hexanes and ethyl acetate. The fractions containing CBG were evaporated to afford a white crystalline solid (330 mg, 56% yield). As confirmed by NMR.

CBG (100 mg, 0.316 mmol) was dissolved in toluene and left open tot air, whereupon tBuOK (100 mg, 0.884 mmol, 2.8 eq) was added to give a deep purple solution. The reaction was monitored via LCMS and had gone to completion in 5 minutes. The reaction mixture was washed with HCl and the aqueous layer was washed with ethyl acetate. The organic layers were concentrated in vacuo and purified via flash chromatography eluted with hexanes and ethyl acetate to give 55 mg of CBGQ. Scheme 1 herein depicts such a process.

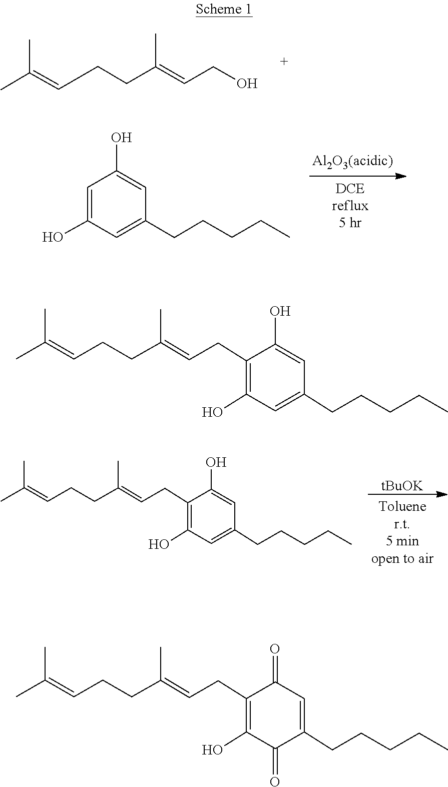

### Example 2: Alternative Synthesis Scheme A

A 4 mL vial with septa was charged with quinone SM (15 mg, 0.045 mmol), DCM (1 mL, 0.05M), followed by 2,6-lutidine (30 μL, 0.261 mmol, 5.5 eq) under argon. The reaction was cooled to 0 C added TBSOTf in (14 uL in 100 ul of DCM dropwise—prepared in a separate vial under argon) was added to the reaction dropwise at 0 C. The reaction stirred at 0° C. for 30 min. The reaction was washed with HNaCO3 (5 mL). The aqueous layer was extracted with EtOAc (3 mL), and the combined organic layers were dried over Na2SO4 and concentrated in vacuo to give a crude dark orange. Scheme 2, shown below, depicts such a process.

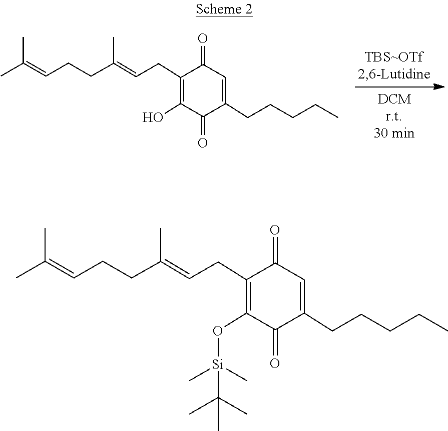

The TBS protected hydroxyquinone (16 mg) was dissolved in pyridine (1 ml) and acetic anhydride (0.5 ml) and left at room temp overnight. The solution was then concentrated in vacuo and dissolved in acetic anhydride (1 mL) and acetic acid (1 mL). Zinc (0.5 g) was added and the mixture was boiled under reflux for 30 min. The residue was filtered through a Celite plug, pyridine (2 ml) was used to wash the solid and the solution was left at room temp overnight after which it was poured into ice water and extracted with ethyl acetate (2×10 ml). Scheme 3, shown below, depicts such a process.

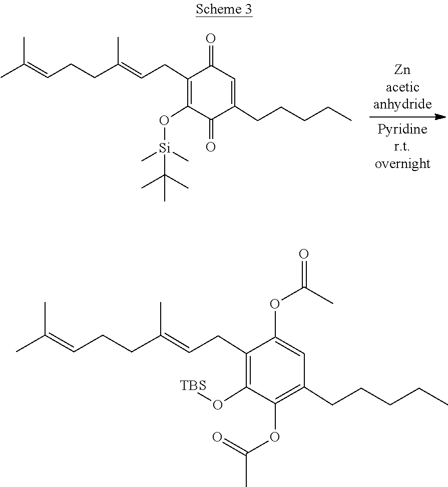

To a solution of the acetylated product (10 mg) in THF (0.5 mL) was added TBAF (20 microL, 1M solution in THF). The solution was stirred at r.t for 1 h whereupon it was concentrated in vacuo and submitted for HPLC purification. Scheme 4, shown below, depicts such a process.

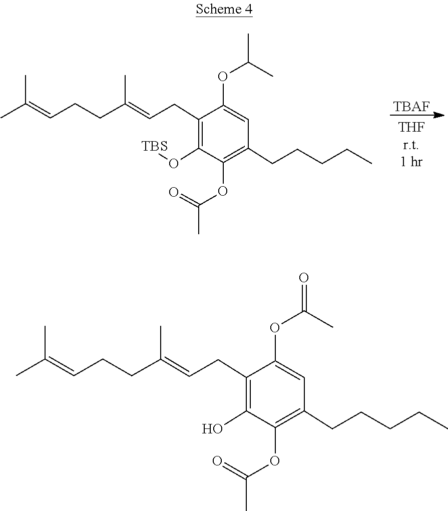

### Example 3: Alternative Synthesis Scheme B

To a solution of CBG (20 mg) in DCM (5 mL) was added 1-fluoropyridinium triflate (13 mg) and was let stir at room temperature overnight. The reaction was monitored by LCMS and when starting material was no longer present, the reaction was concentrated in vacuo and submitted for HPLC purification. Scheme 5 herein depicts such a process.

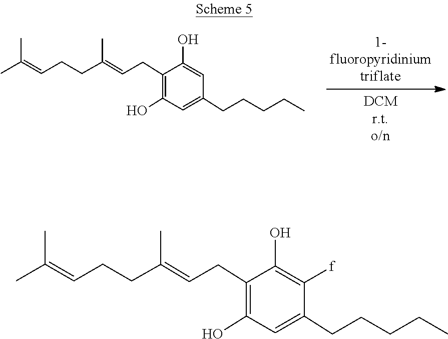

Fluoro-CBG (10 mg, 0.0335 mmol) was dissolved in toluene and left open tot air, whereupon tBuOK (10 mg, 0.093 mmol, 2.8 eq) was added to give a deep purple solution. The reaction was monitored via LCMS and had gone to completion in 5 minutes. The reaction mixture was washed with HCl and the aqueous layer was washed with ethyl acetate. The organic layers were concentrated in vacuo and purified via flash chromatography eluted with hexanes and ethyl acetate to give 5 mg of Fluoro-CBGQ. Scheme 6, shown below, depicts such a process.

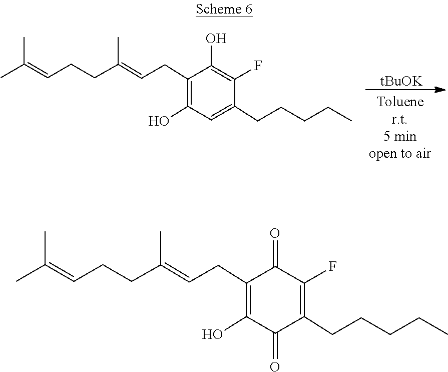

### Example 4: Alternative Synthesis Scheme C

To a solution of CBGQ (20 mg, 0.060 mmol) in ethyl acetate (5 mL) was added sodium dithionite (42 mg, 0.24 mmol) and was let stir at room for 30 min under inert gas. The reaction was monitored by LCMS and when starting material was no longer present, the reaction was concentrated in vacuo and submitted for HPLC purification. Scheme 7, shown below, depicts such a process.

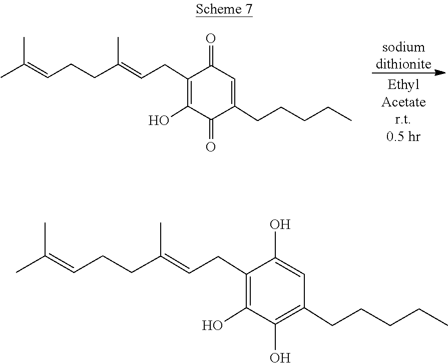

### Example 5: Synthesis of Compounds of Interest

Additional compounds within the scope of formula (I) are prepared according to similar syntheses described herein. Non-limiting examples of additional compounds, designated Compounds IX-XL, are shown in Table 1.

TABLE 1
Non-Limiting Examples of Compounds

| Formula | Structure |
|---|---|
| IX | 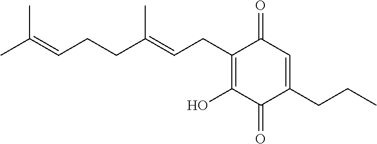 [SMILES: CCCC1=CC(=O)C(C/C=C(\C)CCC=C(C)C)=C(O)C1=O] |
| X | 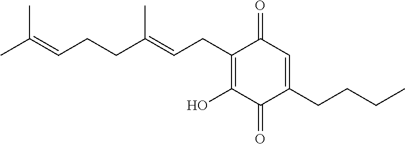 [SMILES: CCCCC1=CC(=O)C(C/C=C(\C)CCC=C(C)C)=C(O)C1=O] |
| XI | 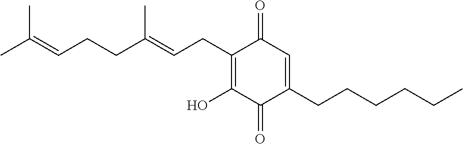 [SMILES: CCCCCCC1=CC(=O)C(C/C=C(\C)CCC=C(C)C)=C(O)C1=O] |
| XII | 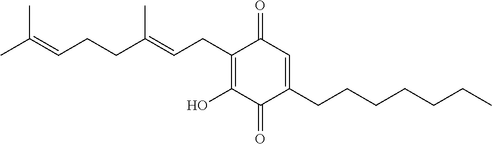 [SMILES: CCCCCCCC1=CC(=O)C(C/C=C(\C)CCC=C(C)C)=C(O)C1=O] |
| XIII | 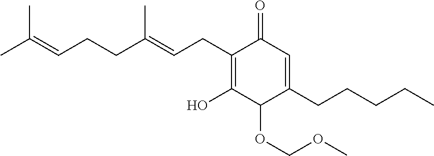 [SMILES: CCCCCC1=CC(=O)C(C/C=C(\C)CCC=C(C)C)=C(O)C1OCOC] |
| XIV | 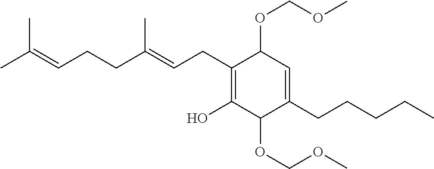 [SMILES: CCCCCC1=CC(OCOC)C(C/C=C(\C)CCC=C(C)C)=C(O)C1OCOC] |
| XV | 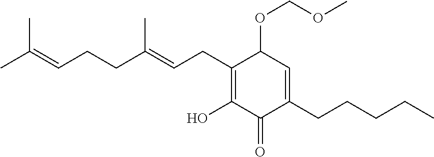 [SMILES: CCCCCC1=CC(OCOC)C(C/C=C(\C)CCC=C(C)C)=C(O)C1=O] |
| XVI | 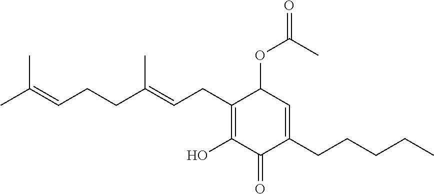 [SMILES: CCCCCC1=CC(OC(C)=O)C(C/C=C(\C)CCC=C(C)C)=C(O)C1=O] |
| XVII | 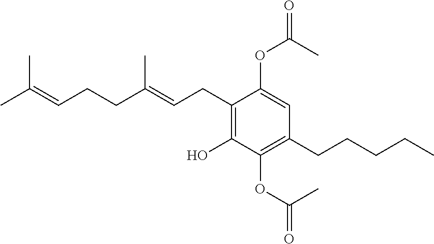 [SMILES: CCCCCc1cc(OC(C)=O)c(C/C=C(\C)CCC=C(C)C)c(O)c1OC(C)=O] |
| XVIII | 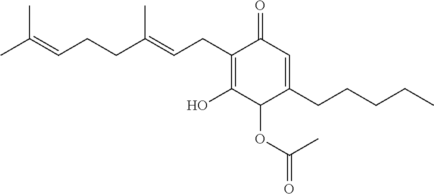 [SMILES: CCCCCC1=CC(=O)C(C/C=C(\C)CCC=C(C)C)=C(O)C1OC(C)=O] |
| XIX | 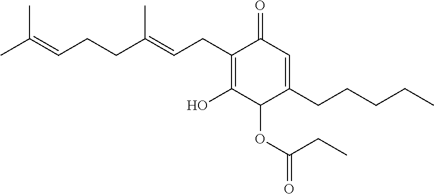 [SMILES: CCCCCC1=CC(=O)C(C/C=C(\C)CCC=C(C)C)=C(O)C1OC(=O)CC] |
| XX | 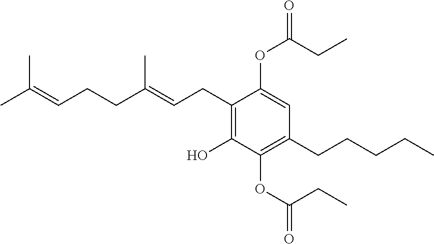 [SMILES: CCCCCc1cc(OC(=O)CC)c(C/C=C(\C)CCC=C(C)C)c(O)c1OC(=O)CC] |
| XXI | 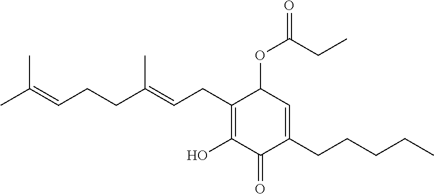 [SMILES: CCCCCC1=CC(OC(=O)CC)C(C/C=C(\C)CCC=C(C)C)=C(O)C1=O] |
| XXII | 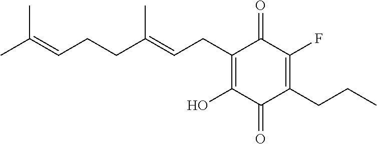 [SMILES: CCCC1=C(F)C(=O)C(C/C=C(\C)CCC=C(C)C)=C(O)C1=O] |
| XXIII | 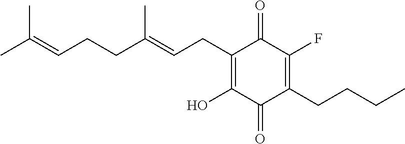 [SMILES: CCCCC1=C(F)C(=O)C(C/C=C(\C)CCC=C(C)C)=C(O)C1=O] |
| XXIV | 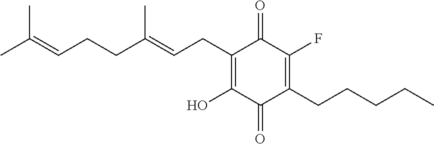 [SMILES: CCCCCC1=C(F)C(=O)C(C/C=C(\C)CCC=C(C)C)=C(O)C1=O] |
| XXV | 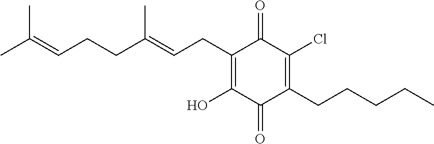 [SMILES: CCCCCC1=C(Cl)C(=O)C(C/C=C(\C)CCC=C(C)C)=C(O)C1=O] |
| XXVI | 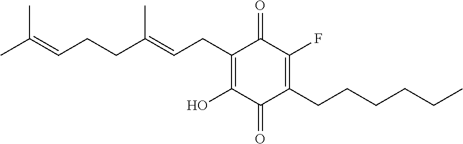 [SMILES: CCCCCCC1=C(F)C(=O)C(C/C=C(\C)CCC=C(C)C)=C(O)C1=O] |
| XXVII | 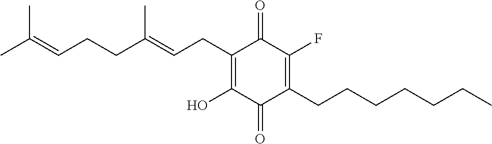 [SMILES: CCCCCCCC1=C(F)C(=O)C(C/C=C(\C)CCC=C(C)C)=C(O)C1=O] |
| XXVIII | 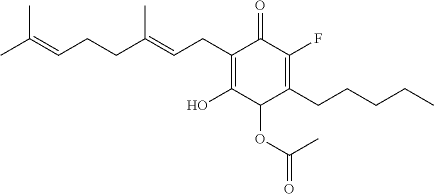 [SMILES: CCCCCC1=C(F)C(=O)C(C/C=C(\C)CCC=C(C)C)=C(O)C1OC(C)=O] |
| XXIX | 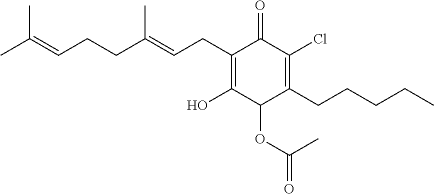 [SMILES: CCCCCC1=C(Cl)C(=O)C(C/C=C(\C)CCC=C(C)C)=C(O)C1OC(C)=O] |
| XXX | 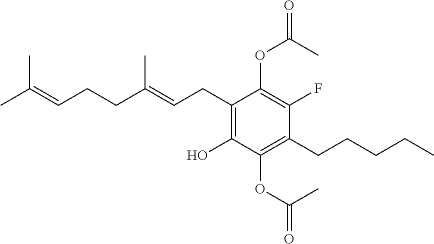 [SMILES: CCCCCc1c(F)c(OC(C)=O)c(C/C=C(\C)CCC=C(C)C)c(O)c1OC(C)=O] |
| XXXI | 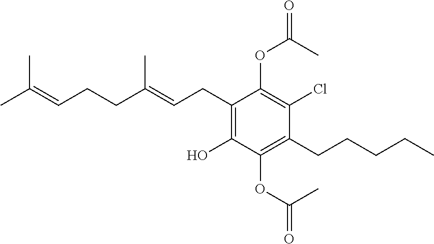 [SMILES: CCCCCc1c(Cl)c(OC(C)=O)c(C/C=C(\C)CCC=C(C)C)c(O)c1OC(C)=O] |
| XXXII | 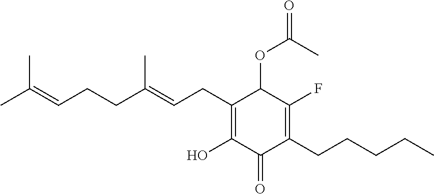 [SMILES: CCCCCC1=C(F)C(OC(C)=O)C(C/C=C(\C)CCC=C(C)C)=C(O)C1=O] |
| XXXIII | 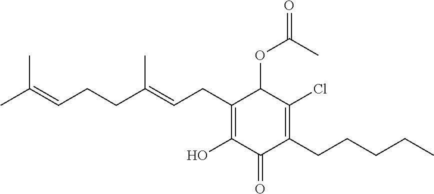 [SMILES: CCCCCC1=C(Cl)C(OC(C)=O)C(C/C=C(\C)CCC=C(C)C)=C(O)C1=O] |
| XXXIV | 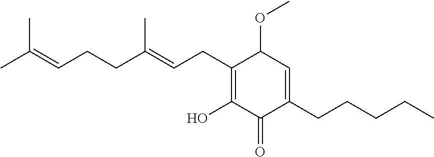 [SMILES: CCCCCC1=CC(OC)C(C/C=C(\C)CCC=C(C)C)=C(O)C1=O] |
| XXXV | 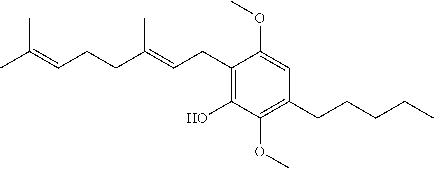 [SMILES: CCCCCc1cc(OC)c(C/C=C(\C)CCC=C(C)C)c(O)c1OC] |
| XXXVI | 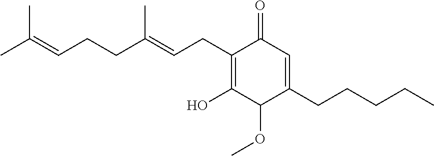 [SMILES: CCCCCC1=CC(=O)C(C/C=C(\C)CCC=C(C)C)=C(O)C1OC] |
| XXXVII | 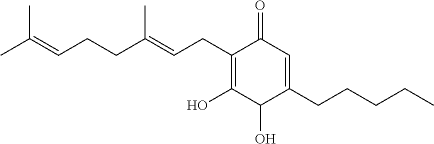 [SMILES: CCCCCC1=CC(=O)C(C/C=C(\C)CCC=C(C)C)=C(O)C1O] |
| XXXVIII | 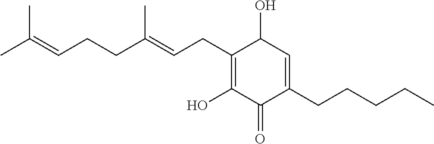 [SMILES: CCCCCC1=CC(O)C(C/C=C(\C)CCC=C(C)C)=C(O)C1=O] |
| XXXIX | 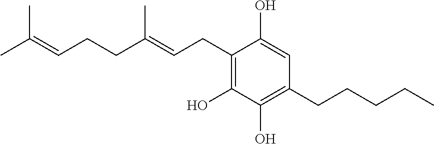 [SMILES: CCCCCc1cc(O)c(C/C=C(\C)CCC=C(C)C)c(O)c1O] |
| XL | 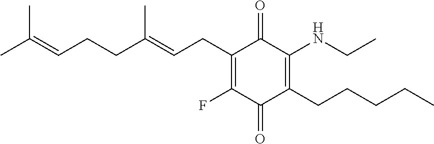 [SMILES: CCCCCC1=C(NCC)C(=O)C(C/C=C(\C)CCC=C(C)C)=C(F)C1=O] |
| XLI | 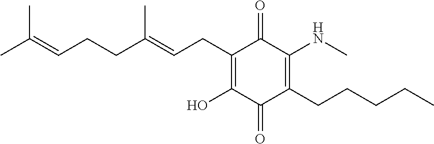 [SMILES: CCCCCC1=C(NC)C(=O)C(C/C=C(\C)CCC=C(C)C)=C(O)C1=O] |
| XLII | 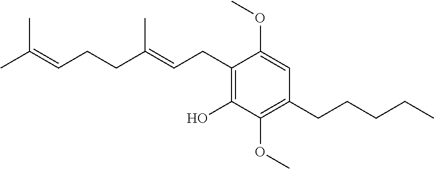 [SMILES: CCCCCc1cc(OC)c(C/C=C(\C)CCC=C(C)C)c(O)c1OC] |
| XLIII | 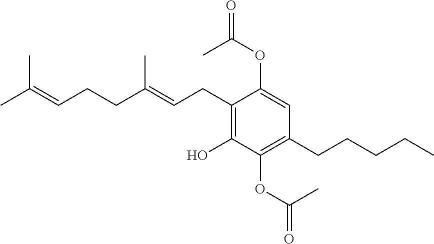 [SMILES: CCCCCc1cc(OC(C)=O)c(C/C=C(\C)CCC=C(C)C)c(O)c1OC(C)=O] |

### Example 6: Compound Activity on Inflammatory/Stress Signaling Molecules

The compounds of Formulae VI, XXXIX, XLII, XLI and XlIII were assessed for inhibitory activity on IFN-gamma, TNF-alpha, IL-6, IL-11B, Lox5, Lox15, and IL10. The compound of Formula VI was also assessed for inhibitory activity on Cox2, as shown in Table 2 herein.

TABLE 2
Inhibitory Activity of Compounds (EC50 μM)

| | | | IL- | | | 5- | 15- | |
|---|---|---|---|---|---|---|---|---|
| | TNF | IL6 | 1β | IFNγ | IL10 | Lox | Lox | Cox2 |
| Formula | 18.5 | 17.9 | 5.3 | 0.91 | 1.1 | 0.27 | 0.98 | 60.6 |
| VI | | | | | | | | |
| Formula | 6.2 | >10 | 0.89 | 1.85 | 1.69 | 0.2 | 2.82 | — |
| XXXIX | | | | | | | | |
| Formula | >10 | >10 | >10 | 4.1 | >10 | 0.21 | 18 | — |
| XLII | | | | | | | | |
| Formula | >10 | >10 | 0.12 | 2.51 | >10 | 0.48 | 28.8 | — |
| XLI | | | | | | | | |
| Formula | 8.68 | >10 | 0.89 | 1.13 | 1.2 | 0.34 | 8.4 | — |
| XLIII | | | | | | | | |

In particular, the compound of Formula VI (Molecular Weight 314.47), showed particularly strong selective activity for IFN-gamma, TNF-alpha, IL-6, IL-IB, Lox5, Lox15, and IL10 over Cox2. For example, the compound of Formula VI had over 200-fold stronger inhibition for 5-Lox compared to Cox-2, and over 60-fold stronger inhibition of 15-Lox and IFNγ compared to Cox-2. All five compounds tested demonstrated strong inhibitory activity on TNF, IL-6, IL-1β, IL-10, 5-Lox, and 15-Lox. Compounds of Formulae XXXIX, XLI, and XLIII had particular strong inhibitory activity on IL-10 compared with the other compounds.

Methods and Materials for each assay were conducted as described below.

### Nanosyn Protocol for Biochemical COX-2 Assay

### Materials:

Human COX2, GenBank Accession No. NM_000963, a.a. 1-604 (end), with C-terminal FLAG-His-tag, MW=71 kDa, expressed in a baculovirus infected Sf9 cell expression system was obtained from BPS Biosciences (Catalog #71111)
Cyclooxygenase (COX) Activity Assay Kit (Fluorometric) was obtained from Abcam (Catalog #ab204699)
Other reagents: black 384 well assay plates were obtained from Corning (Cat #3820).
Equipment: Synergy Neo2 Multi-mode plate reader (Biotek), Labcyte 550 acoustic dispenser (Labcyte), Biomek FX robotic dispenser (Beckman Coulter), Sorvall Legend RT centrifuge (Kendro).

Serially diluted compounds were prepared in DMSO in Echo qualified 384 well plates. An assay buffer was then prepared, and supplemented with Cox cofactor and fluorogenic probe as described in the Cox activity kit manual. 10 μL of the supplemented buffer was dispensed into control (no enzyme) wells in the black 384 well assay plate. Next, 2× enzyme solution was prepared, comprising 10 nM COX-2 enzyme in the supplemented assay buffer. 10 μL of the 2× enzyme solution was dispensed into test wells in the black 384 well assay plate.

Serially diluted compounds were added to the assay plate by acoustic dispensing (Labcyte 550). Control wells received DMSO only. 2× substrate was then prepared, comprising arachidonic acid prepared in the assay buffer according to the Cox activity manual. 10 μL of the 2× substrate was added to all wells in the assay plate. The plate was then incubated for 20 min. Afterwards, the plate was read on a Synergy Neo2 in fluorescence mode: excitation 535 nm, emission 590 nm. % inhibition of the compound was calculated using following equation:

%-Inh=(RFU100%−RFUcompound)/(RFU100%−RFU0%)*100,

where RFU100% is fluorescence in control samples without compound, RFU0% is fluorescence in control samples without COX-2, and RFU compound fluorescence in a sample with compound.

The calculated %-Inh values were then plotted against compound concentration. To determine EC50 values, dose-response curves were fitted by a 4-parameter sigmoid inhibition model using XLFit® software (IDBS).

### Nanosyn Protocol for TNFa Secretion Assay in Human PBMCs

### Materials:

Cells: Cryopreserved normal human peripheral blood mononuclear cells (PBMC) were obtained from AllCells (cat #PB006F).
Cell culture media: RPMI medium was obtained from ThermoFisher (Cat #22400071). Fetal bovine serum (FBS) was obtained from ThermoFisher (Cat #16000044).
Other reagents: 96 well TC plates were obtained from ThermoFisher (Cat #07-2000-90). LPS was obtained from Sigma (Cat #L3024). TNFa ELISA plates were obtained from BD Biosciences (Cat #550610).
Equipment: NuAire CO2 Incubator, Nikon Inverted Microscope, Biomek FX robotic dispenser (Beckman Coulter), Spectramax reader (Molecular Devices), Sorvall Legend RT centrifuge (Kendro), hemocytometer.

TNF-alpha was assessed using a standard ELISA protocol. Cryopreserved PBMCs were thawed in a water bath and transferred to 50 mL polypropylene tubes. The cells were washed two times with 25 mL of RPMI medium supplemented with 5% of heat-inactivated FBS and re-suspended at a density of 4.5\hat{ }⁶ cells/mL. The cells were plated into 96 well TC late (100 uL/well). Serially diluted compound was added to the cells in duplicate wells for each concentration. Control wells received DMSO only (0.3% final). The cells were pre-incubated with compound for 1 hr. After pre-incubation, the cells were stimulated by LPS (100 ng/mL) for 6 hr. Control samples with and without LPS in the absence of compound were assembled in each test plate. TNFa was detected in media supernatants by using a sandwich anti-TNFa ELISA kit (BD Biosciences) according to protocol provided by the manufacturer. ELISA readout (optical density at 450 nm) is commensurable with the amount of TNFa produced by the cells. Assay window was determined in each experiment by comparing average OD450 values in stimulated and un-stimulated samples in the absence of compound. ELISA calibration was performed in each detection plate using serially diluted recombinant human TNFa, to ensure that OD450 values for all test samples were within linear range of detection.%-Inhibition by compound was calculated using following equation:

$$\% - Inh = ( OD {450}_{100 \%} - OD {450}_{compound} ) / ( OD {450}_{100 \%} - OD {450}_{0 \%} ) * 100 ,$$

where OD450_{100%} is optical density in control samples stimulated with LPS without compound, OD450_{0%} is optical density in unstimulated control samples, and OD450_{compound} is optical density in a sample stimulated with LPS in the presence of compound.

The calculated %-Inh values were plotted against compound concentration. To determine IC50 values, dose-response curves were fitted by a 4-parameter sigmoid inhibition model using XLFit® software (IDBS).

### Nanosvyn Protocol for IFNgamma Secretion Assay in Human PBMCs

### Materials:

Cells: Cryopreserved normal human peripheral blood mononuclear cells (PBMC) were obtained from AllCells (cat #PB006F).
Cell culture media: RPMI medium was obtained from ThermoFisher (Cat #22400071). Fetal bovine serum (FBS) was obtained from ThermoFisher (Cat #16000044).
Other reagents: 96 well TC plates were obtained from ThermoFisher (Cat #07-2000-90). Phytohemagglutinin-M (PHA-M) was obtained from Millipore/Sigma (Cat #11082132001). BD human IFN-g ELISA kit II was obtained from BD Biosciences (Cat #550612).
Equipment: NuAire CO2 Incubator, Nikon Inverted Microscope, Biomek FX robotic dispenser (Beckman Coulter), Spectramax reader (Molecular Devices), Sorvall Legend RT centrifuge (Kendro), hemocytometer.

IFN-gamma was assessed using a standard ELISA protocol. Cryopreserved PBMCs were thawed in a water bath and transferred to 50 mL polypropylene tubes. The cells were washed two times with 25 mL of RPMI medium supplemented with 5% of regular FBS and re-suspended at a density of 1.5\hat{ }⁶ cells/mL. The cells were plated into 96 well TC late (100 uL/well). Serially diluted compound was added to the cells in duplicate wells for each concentration. Control wells received DMSO only (0.3% final). The cells were pre-incubated with compound for 2 hr. After pre-incubation, the cells were stimulated by PHA-M (10 ug/mL) for 40 hr. Control samples with and without PHA-M in the absence of compound were assembled in each test plate. IFNg was detected in media supernatants by using a sandwich anti-IFNg ELISA kit (BD Biosciences) according to protocol provided by the manufacturer. ELISA readout (optical density at 450 nm) is commensurable with the amount of IFNg produced by the cells. Assay window was determined in each experiment by comparing average OD450 values in stimulated and un-stimulated samples in the absence of compound. ELISA calibration was performed in each detection plate using serially diluted recombinant human IFN gamma, to ensure that OD450 values for all test samples were within linear range of detection. %-Inhibition by compound was calculated using following equation:

$$\% - Inh = ( OD {450}_{100 \%} - OD {450}_{compound} ) / ( OD {450}_{100 \%} - OD {450}_{0 \%} ) * 100 ,$$

where OD450_{100%} is optical density in control samples stimulated with PHA-M without compound, OD450_{0%} is optical density in unstimulated control samples, and OD450_{compound} is optical density in a sample stimulated with PHA-M in the presence of compound.

The calculated %-Inh values were plotted against compound concentration. To determine IC50 values, dose-response curves were fitted by a 4-parameter sigmoid inhibition model using XLFit® software (IDBS).

### Nanosyn Protocol for IL-6 Secretion Assay in Human PBMKs

### Materials:

Cells: Cryopreserved normal human peripheral blood mononuclear cells (PBMC) were obtained from AllCells (cat #PB006F).
Cell culture media: RPMI medium was obtained from ThermoFisher (Cat #22400071). Fetal bovine serum (FBS) was obtained from ThermoFisher (Cat #16000044).
Other reagents: 96 well TC plates were obtained from ThermoFisher (Cat #07-2000-90). LPS was obtained from Sigma (Cat #L3024). BD OptEIA human IL-6 ELISA plates were obtained from BD Biosciences (Cat #550799).
Equipment: NuAire CO2 Incubator, Nikon Inverted Microscope, Biomek FX robotic dispenser (Beckman Coulter), Spectramax reader (Molecular Devices), Sorvall Legend RT centrifuge (Kendro), hemocytometer.

IL-6 was assessed using a standard ELISA protocol. Cryopreserved PBMCs were thawed in a water bath and transferred to 50 mL polypropylene tubes. The cells were washed two times with 25 mL of RPMI medium supplemented with 5% of heat-inactivated FBS and re-suspended at a density of 4.5\hat{ }⁶ cells/mL. The cells were plated into 96 well TC late (100 uL/well). Serially diluted compound was added to the cells in duplicate wells for each concentration. Control wells received DMSO only (0.3% final). The cells were pre-incubated with compound for 1 hr. After pre-incubation, the cells were stimulated by LPS (100 ng/mL) for 6 hr. Control samples with and without LPS in the absence of compound were assembled in each test plate. IL-6 was detected in media supernatants by using a sandwich anti-IL-6 ELISA kit (BD Biosciences) according to protocol provided by the manufacturer. ELISA readout (optical density at 450 nm) is commensurable with the amount of IL-6 produced by the cells. Assay window was determined in each experiment by comparing average OD450 values in stimulated and un-stimulated samples in the absence of compound. ELISA calibration was performed in each detection plate using serially diluted recombinant human IL-6, to ensure that OD450 values for all test samples were within linear range of detection.%-Inhibition by compound was calculated using following equation.

$$\% - Inh = ( OD {450}_{100 \%} - OD {450}_{compound} ) / ( OD {450}_{100 \%} - OD {450}_{0 \%} ) * 100 ,$$

where OD450₁₀₀% is optical density in control samples stimulated with LPS without compound, OD450_{0%} is optical density in unstimulated control samples, and OD450_{compound} is optical density in a sample stimulated with LPS in the presence of compound.

The calculated %-Inh values were plotted against compound concentration. To determine IC50 values, dose-response curves were fitted by a 4-parameter sigmoid inhibition model using XLFit® software (IDBS).

### Nanosyn Protocol for Biochemical LOX-5 Assay

### Materials:

Active recombinant human LOX-5 protein expressed in insect cells (Amino Acids 1-674) was obtained from Cayman (Cat #60402)
2′,7′-dichlorodihydrofluorescein diacetate, H2DCFDA, was obtained from Sigma (Cat #96883)
Arachidonic acid, AA, was obtained from Sigma (Cat #A3611)
Other reagents: black 384 well assay plates were obtained from Corning (Cat #3820).
Equipment: Synergy Neo2 Multi-mode plate reader (Biotek), Labcyte 550 acoustic dispenser (Labcyte), Biomek FX robotic dispenser (Beckman Coulter), Sorvall Legend RT centrifuge (Kendro).

The LOX-5 assay was performed in assay buffer comprising: 50 mM Tris (pH 7.5), 2 mM EDTA, 2 mM CaCl2), 3 uM AA, 10 uM ATP, 10 uM H2DCFDA, and recombinant enzyme (50 mU per 20 ul reaction, as defined by manufacturer). Serially diluted compounds were prepared in DMSO in Echo qualified 384 well plates. An assay buffer was then prepared without AA and ATP supplemented with 20 uM H2DCFDA. 10 uL of the supplemented buffer was dispensed into control (no enzyme) wells in the black 384 well assay plate. Next, a 2× enzyme solution was prepared, comprising 100 mU LOX-5 enzyme in the supplemented assay buffer. 10 uL of the 2× enzyme solution was then dispensed into test wells in the black 384 well assay plate. Serially diluted compounds were added to the assay plate by acoustic dispensing (Labcyte 550). Control wells received DMSO only.

Next, the 2× substrate was prepared to comprise 6 uM arachidonic acid and 20 uM ATP in the assay buffer. 10 uL of the 2× substrate was then added to all wells in the assay plate. The plate was incubated for 20 min. The plate was read on Synergy Neo2 in fluorescence mode: excitation 485 nm, emission 530 nm. % inhibition and the resulting EC50 was calculated for the compound using the following equation:

$$\% - Inh = ( RFU 100 \% - RFUcompound ) / ( RFU 100 \% - RFU 0 \% ) * 100 ,$$

where RFU100% is fluorescence in control samples without compound, RFU0% is fluorescence in control samples without LOX-5, and RFUcompound fluorescence in a sample with compound.

The calculated %-Inh values were plotted against compound concentration. To determine EC50 values, dose-response curves were fitted by a 4-parameter sigmoid inhibition model using XLFit® software (IDBS).

### Nanosyn Protocol for Biochemical LOX-15 Assay

### Materials:

Recombinant human ALOX15 (Arachidonate 15-Lipoxygenase), full length encompassing amino acids 2-662(end). In this construct the N-terminal end of ALOX15 is fused with a FLAG tag was obtained from BPS Biosciences (Cat #50220).
Dihydrorhodamine 123, DHR, was obtained from Sigma (Cat #1054)
Arachidonic acid, AA, was obtained from Sigma (Cat #A3611)
Other reagents: black 384 well assay plates were obtained from Corning (Cat #3820).
Equipment: Synergy Neo2 Multi-mode plate reader (Biotek), Labcyte 550 acoustic dispenser (Labcyte), Biomek FX robotic dispenser (Beckman Coulter), Sorvall Legend RT centrifuge (Kendro).

The LOX-15 assay was performed in assay buffer comprising: 50 mM Tris (pH 7.5), 5 uM DHR, 50 uM AA, and 50 nM recombinant enzyme. Serially diluted compounds were prepared in DMSO in Echo qualified 384 well plates. An assay buffer was then prepared without supplemented 10 uM DHRA. 10 uL of the supplemented buffer was dispensed into control (no enzyme) wells in the black 384 well assay plate. Next, a 2× enzyme solution was prepared, comprising 100 nM LOX-15 enzyme in the supplemented assay buffer. 10 μL of the 2× enzyme solution was then dispensed into test wells in the black 384 well assay plate. Serially diluted compounds were added to the assay plate by acoustic dispensing (Labcyte 550). Control wells received DMSO only.

Next, the 2× substrate was prepared to comprise 100 uM arachidonic acid in the assay buffer. 10 uL of the 2× substrate was then added to all wells in the assay plate. The plate was incubated for 3 hours. The plate was read on Synergy Neo2 in fluorescence mode: excitation 485 nm, emission 530 nm. % inhibition and the resulting EC50 was calculated for the compound using the following equation:

$$\% - Inh = ( RFU 100 \% - RFUcompound ) / ( RFU 100 \% - RFU 0 \% ) * 100 ,$$

where RFU100% is fluorescence in control samples without compound, RFU0% is fluorescence in control samples without LOX-15, and RFUcompound fluorescence in a sample with compound.

The calculated %-Inh values were plotted against compound concentration. To determine EC50 values, dose-response curves were fitted by a 4-parameter sigmoid inhibition model using XLFit® software (IDBS).

Although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the invention.

### Nanosyn Protocol for IL10 Secretion Assay in Human PBMCs

### Materials:

Cells: Cryopreserved normal human peripheral blood mononuclear cells (PBMC) were obtained from AllCells (cat #PB006F).
Cell culture media: RPMI medium was obtained from ThermoFisher (Cat #22400071). Fetal bovine serum (FBS) was obtained from ThermoFisher (Cat #16000044).
Other reagents: 96 well TC plates were obtained from ThermoFisher (Cat #07-2000-90). Phytohemagglutinin-M (PHA-M) was obtained from Millipore/Sigma (Cat #11082132001). BD human IFN-g ELISA kit II was obtained from BD Biosciences (Cat #550612).
Equipment: NuAire CO2 Incubator, Nikon Inverted Microscope, Biomek FX robotic dispenser (Beckman Coulter), Spectramax reader (Molecular Devices), Sorvall Legend RT centrifuge (Kendro), hemocytometer.

Cryopreserved PBMCs were thawed in a water bath and transferred to 50 mL polypropylene tubes. The cells were washed two times with 25 mL of RPMI medium supplemented with 5% of regular FBS and re-suspended at a density of 1.5\hat{ }⁶ cells/mL. The cells were plated into 96 well TC late (100 uL/well). Serially diluted compound was added to the cells in duplicate wells for each concentration. Control wells received DMSO only (0.3% final). The cells were pre-incubated with compound for 2 hr. After pre-incubation, the cells were stimulated by PHA-M (10 ug/mL) for 40 hr. Control samples with and without PHA-M in the absence of compound were assembled in each test plate. 1110 was detected in media supernatants by using a sandwich anti-IL10 ELISA kit (BD Biosciences) according to protocol provided by the manufacturer. ELISA readout (optical density at 450 nm) is commensurable with the amount of IL10 produced by the cells. Assay window was determined in each experiment by comparing average OD450 values in stimulated and un-stimulated samples in the absence of compound. ELISA calibration was performed in each detection plate using serially diluted recombinant human IL10 standard, to ensure that OD450 values for all test samples were within linear range of detection.%-Inhibition by compound was calculated using following equation:

$$\% - Inh = ( OD {450}_{100 \%} - OD {450}_{compound} ) / ( OD {450}_{100 \%} - OD {450}_{0 \%} ) * 100 ,$$

where OD450_{100%} is optical density in control samples stimulated with PHA-M without compound, OD450_{0%} is optical density in unstimulated control samples, and OD450_{compound} is optical density in a sample stimulated with PHA-M in the presence of compound. The calculated %-Inh values were plotted against compound concentration. To determine IC50 values, dose-response curves were fitted by a 4-parameter sigmoid inhibition model using XLFit® software (IDBS).

### Example 7: SK-MEL-28 Cell Line Toxicity

The compounds of interest were assessed cell viability in the SK-MEL-28 melanoma cell line (ATCC® HTB-72). The cells were cultured in the EMEM medium supplemented with 10% FBS, in the temperature of 37° C., 5% CO₂ and 95% humidity. Culture medium were purchased from ATCC, USA. Cells were incubated for a total of 72 hours, using staurosporine as a control compound.

### Materials and Reagents:

General cell culture reagents and plastic.
384-Well Flat white Poly-styrene TC-Treated Microplates (Corning).
384 well poly-propylene plates (acoustic grade, Labcyte) for compound dilution.
Cell titer Glo luminescent Cell Viability reagent (Cat #G7571, Promega).

### Equipment:

Synergy Neo2 Multi-mode plate reader (Biotek)
NuAir CO₂ Incubator
Nikon Inverted Microscope
Labcyte 550 acoustic dispenser (Labcyte)
Biomek FX robotic dispenser (Beckman Coulter)
Sorvall Legend RT centrifuge (Kendro)
Hemocytometer

### Experimental Methodology:

The half maximal inhibition concentration (IC50) for each compound was determined by harvesting cells during the logarithmic growth period and then counting cell number using hemocytometer. The cell concentrations were adjusted to 5×10⁴ cells/mL with respective culture medium. 30 μL of cell suspensions were added to three 384-well plates with the final cell density of 1.5×10³ cells/well. Then, using a Biomek FX, test articles were serially diluted (10 mM top, 3× dilution intervals) along with a reference inhibitor (1 mM top, 3× dilution intervals) in acoustic grade Labcyte plate. Then, using an acoustic dispenser (Labcyte550), compounds were added as serially diluted to plate with cells: 90 nL of serially diluted compounds per well. 90 nL DMSO is added to control wells. Test plates were incubated for 72 h in the humidified incubator at 37° C. with 5% CO₂. 25 μL of reconstituted Cell titer Glo reagent was added to each well. Each test plate was then incubated for 20 min at room temperature. Luminescence was then recorded using Synergy Neo2Multi-mode Reader.

In order to calculate intersect IC50, a dose-response curve was fitted using nonlinear regression model with a sigmoidal dose response.

The formula for calculating surviving rate is shown below, and the intersect IC50 was calculated according to the dose-response curve generated by the software:

$$The surviving rate ( \% - inh ) = ( {RLU}_{DMSO article} - {RLU}_{Test article} ) \text{} / ( {RLU}_{DMSO control} - {RLU}_{Medium only control} ) \times 100 \% .$$

### Results:

The compound of Formula VI was found to have an EC50 of >30 uM. In other words, SK-MEL-28 cells have more than 250-fold higher tolerance to the compound of Formula VI than staurosporine, which had an IC50 of about 0.21 uM. The results of the assay are as shown in the below Table 3.

TABLE 3
SK-MEL-28 cell viability with increased compound exposure

Cell Viability (%)

| | | Compound of | |
|---|---|---|---|
| | | Formula VI | Staurosporine |
| Concentration | 30 | 83.2 | 8.85 |
| of | 10 | 99.0 | 8.55 |
| Compound | 3.3 | 104.0 | 17.3 |
| (uM) | 1.1 | 99.9 | 33.0 |
| | 0.37 | 101.0 | 41.1 |
| | 0.12 | 100.0 | 53.9 |
| | 0.041 | 99.9 | 70.0 |
| | 0.014 | 98.5 | 92.9 |
| | 0.0046 | 101.0 | 94.1 |
| | 0.0015 | 96.2 | 96.7 |
| | 0.00051 | 97.2 | 94.9 |
| | 0.00017 | 100.0 | 96.2 |

### Example 8: PC3 Cell Line Toxicity

The compounds of interest were assessed cell viability in the PC3 prostate adenocarcenoma cell line (ATCC® CRL-1435). The cells were cultured in the F12K medium supplemented with 10% FBS, in the temperature of 37° C., 5% CO₂ and 95% humidity. Culture medium were purchased from ATCC, USA. Cells were incubated for a total of 72 hours, using staurosporine as a control compound. The materials, reagents, and experimental methodology were the same as in Example 7.

### Results:

The compound of Formula VI was found to have an EC50 of >30 uM. In other words, PC3 cells have more than 10,000-fold higher tolerance to the compound of Formula VI than staurosporine, which had an IC50 of about 0.004 uM. The results of the assay are as shown in the below Table 4.

TABLE 4
PC3 cell viability with increased compound exposure

Cell Viability (%)

| | | Compound of | |
|---|---|---|---|
| | | Formula VI | Staurosporine |
| Concentration | 30 | 84.5 | 7.1 |
| of | 10 | 91.2 | 6.8 |
| Compound | 3.3 | 94.6 | 8.1 |
| (uM) | 1.1 | 94.0 | 8.4 |
| | 0.37 | 97.7 | 9.5 |
| | 0.12 | 96.0 | 10.3 |
| | 0.041 | 95.9 | 12.7 |
| | 0.014 | 96.5 | 34.5 |
| | 0.0046 | 96.9 | 43.2 |
| | 0.0015 | 96.0 | 67.1 |
| | 0.00051 | 100.9 | 92.2 |
| | 0.00017 | 97.4 | 102.0 |

### Example 9: HL-60 Cell Line Toxicity

The compounds of interest were assessed cell viability in the HL-60 acute promyelocytic leukemia cell line (ATCC® CCL-240). The cells were cultured in the IMDM supplemented with 10% FBS, in the temperature of 37° C., 5% CO₂ and 95% humidity. Culture medium were purchased from ATCC, USA. Cells were incubated for a total of 72 hours, using staurosporine as a control compound. The materials, reagents, and experimental methodology were the same as in Example 7.

### Results:

The compound of Formula VI was found to have an EC50 of >30 uM. In other words, HL-60 cells have more than 1,500-fold higher tolerance to the compound of Formula VI than staurosporine, which had an IC50 of about 0.03 uM. The results of the assay are as shown in the below Table 5.

TABLE 5
HL-60 cell viability with increased compound exposure

Cell Viability (%)

| | | Compound of | |
|---|---|---|---|
| | | Formula VI | Staurosporine |
| Concentration | 30 | 61.5 | 9.6 |
| of | 10 | 81.9 | 10.4 |
| Compound | 3.3 | 97.8 | 11.7 |
| (uM) | 1.1 | 91.3 | 13.9 |
| | 0.37 | 91.3 | 18.7 |
| | 0.12 | 85.1 | 27.7 |
| | 0.041 | 83.6 | 36.3 |
| | 0.014 | 94.1 | 66.6 |
| | 0.0046 | 94.0 | 90.4 |
| | 0.0015 | 91.6 | 98.3 |
| | 0.00051 | 113.4 | 96.5 |
| | 0.00017 | 102.3 | 98.3 |

### Example 10; PANC-1 Cell Line Toxicity

The compounds of interest were assessed cell viability in the PANC-1 pancreatic carcinoma cell line (ATCC® CRL-1469). The cells were cultured in DMEM supplemented with 10% FBS, in the temperature of 37° C., 5% CO₂ and 95% humidity. Culture medium were purchased from ATCC, USA. Cells were incubated for a total of 72 hours, using staurosporine as a control compound. The materials, reagents, and experimental methodology were the same as in Example 7.

### Results:

The compound of Formula VI was found to have an EC50 of >30 uM. In other words, PANG-1 cells have more than 750-fold higher tolerance to the compound of Formula VI than staurosporine, which had an IC50 of about 0.08 uM. The results of the assay are as shown in the below Table 6.

TABLE 6
PANC-1 cell viability with increased compound exposure

Cell Viability (%)

| | | Compound of | |
|---|---|---|---|
| | | Formula VI | Staurosporine |
| Concentration | 30 | 99.8 | 8.9 |
| of | 10 | 99.4 | 10.3 |
| Compound | 3.3 | 95.7 | 12.2 |
| (uM) | 1.1 | 102.6 | 24.5 |
| | 0.37 | 98.6 | 31.0 |
| | 0.12 | 99.2 | 49.7 |
| | 0.041 | 99.9 | 52.8 |
| | 0.014 | 100.2 | 73.7 |
| | 0.0046 | 104.2 | 86.2 |
| | 0.0015 | 100.4 | 95.5 |
| | 0.00051 | 107.5 | 96.0 |
| | 0.00017 | 106.6 | 96.7 |

### Example 11: CB1 and CB2 Receptor Agonists Assays

The compound with Formula VI was assayed for its ability to act as an agonist against the CB1 and CB2 receptors. The assays were performed in cAMP Hunter eXpress cells engineered to overexpress naturally Gi/Go-coupled wildtype cannabinoid receptor 1 (CNR1) or cannabinoid receptor 2 (CNR2) in CHO-K1 cell type. In these cells, CB1 and/or CB32 agonists inhibit production of intracellular cAMP in response to stimulation with Forskolin. cAMP levels are determined using luminogenic detection reagents provided with the Hunter eXpress cAMP detection kit.

### Materials:

Assay ready CNR1 CHO-K1 cells were obtained from Eurofins/Discover X, cat #95-0071E2
Assay ready CNR2 CHO-K1 cells were obtained from Eurofins/Discover X, cat #95-0183E2
Hunter eXpress cAMP detection kit was also from Discover X. The kit includes all cAMP detection reagents, Forskolin, complete cell plating medium and cell assay buffer.

### Protocol:

The Assay-Ready CNR1 CHO-K1 or CNR2 CHO-K1 cells were thawed and re-suspended in pre-warmed Assay Complete plating medium and inoculated into 96 well plate at a density of 30,000 cells in 100 μL. The plates with cells were incubated for 24 h at 37° C. in 5% CO2. After incubation, plating media was aspirated and replaced with 30 μL of cell assay buffer and 15 μL of the assay buffer supplemented with 75 uM Forskolin and serially diluted compounds. The cells were stimulated with the Forskolin/compound buffer for 30 min. After stimulation, 15 μL of cAMP antibody reagent and 60 μL of cAMP working detection solution were added to the cells and, the cells were incubated for 1 h at room temperature. After 1 h incubation, 60 μL of cAMP Solution A was added to the wells and the plate was incubated for 3 h at room temperature. The plate was read on Biotek Synergy Neo2 reader in luminescent mode. CB1 and CB2 agonists should reduce cAMP production in response to Forskilin. Relative luminescence units (RLUs) were plotted against compound concentration to determine apparent EC50 values of the agonist compounds.

### Results:

The Compound with Formula VI demonstrated good selectivity as an agonist for CB2 receptor activation over CB1 receptor activation. The results of this assay with test Compound of Formula VI, along with control compounds 11-hydroxy-THC and CBD, are given in the below Table 7:

TABLE 7
Compound Agonist IC50 Values

| | CB1 Receptor | CB2 Receptor | Selectivity for CB2 |
|---|---|---|---|
| Compound | IC50 (uM) | IC50 (uM) | over CB1 (fold) |
| Formula VI | >3 | 0.12 | >25 |
| CBD | 1.85 | 3.1 | 0.6 |
| 11-hydroxy-THC | 0.001 | 0.051 | 0.02 |

### Summary

The Compound of Formula VI has shown potent anti-inflammatory properties in the phenotypic inflammation assays. It has also demonstrated high potency and selectivity for the CB2 receptor activation over the CB1 receptor as compared to standard cannabinoids like THC and CBD.

### Example 12: Synthesis of the Compound of Formula XXXIX

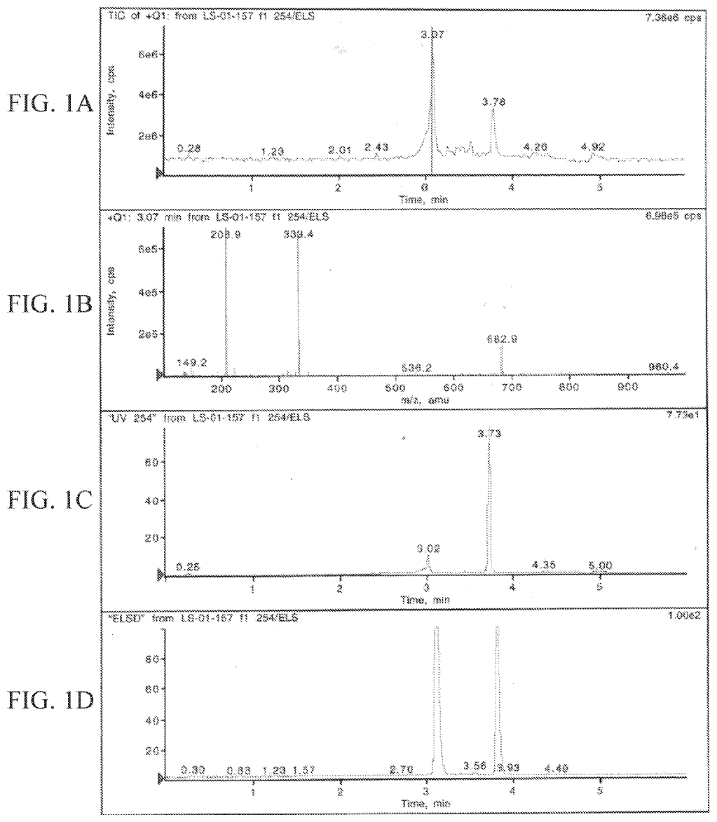

To a solution of CBGQ (20 mg, 0.060 mmol) in ethyl acetate (5 mL) was added sodium dithionite (42 mg, 0.24 mmol) and the mixture was stirred at room temperature for 30 min under inert gas. The reaction was monitored by LCMS and when starting material was no longer present, the reaction was concentrated in vacuo and submitted for HPLC purification. The resulting product was 3.3 mg, 16.5% yield. [M+H]⁺ 333.4. FIGS. 1A-1D show the gas chromatography mass spectrometry (GC-MS) total ion chromatogram (TIC) spectra, the chromatography mass spectrometry (GC-MS) mass-to-charge ratio (m/z) spectra, the high-performance liquid chromatography ultraviolet (HPLC-UV) spectra at 254 nm, and the high-performance liquid chromatography evaporative light scattering detector (HPLC-ELSD) spectra for a compound of Formula XXXIX, respectively. Scheme 8, shown below, depicts such a process.

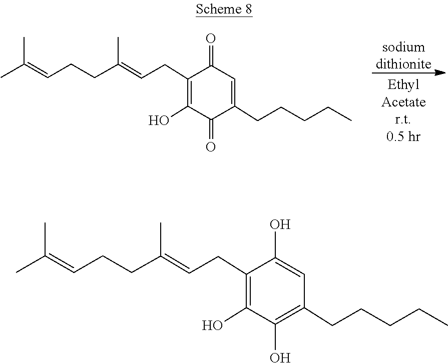

### Example 13: Synthesis of the Compound of Formula XLI

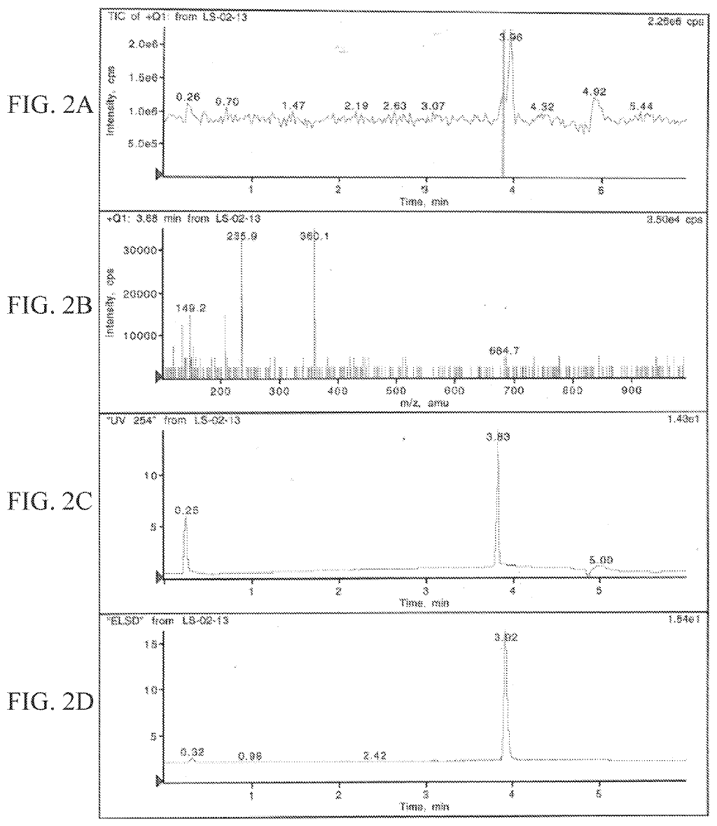

CBGQ (20 mg, 0.060 mmol) was charged to a vial whereupon 2M H₂NMe in excess in THF was added (2 mL). The reaction was allowed to stir for ten minutes, then 1M HCl solution was added. The reaction was allowed to stir ten minutes then concentrated in vacuo and submitted for HPLC purification. The resulting product was 1.2 mg, 5.5% yield. [M+H]⁺ 360.1. FIGS. 2A-2D show the gas chromatography mass spectrometry (GC-MS) total ion chromatogram (TIC) spectra, the chromatography mass spectrometry (GC-MS) mass-to-charge ratio (m/z) spectra, the high-performance liquid chromatography ultraviolet (HPLC-UV) spectra at 254 nm, and the high-performance liquid chromatography evaporative light scattering detector (HPLC-ELSD) spectra for a compound of Formula XLI, respectively. Scheme 9, shown below, depicts such a process.

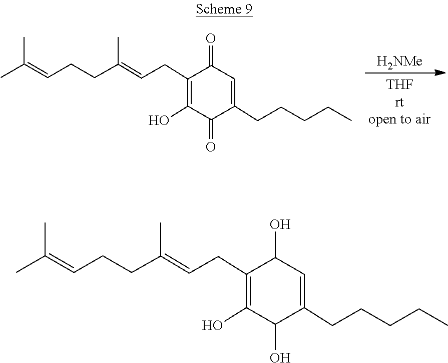

### Example 14: Synthesis of the Compound of Formula XLII

CBGQ (20 mg, 0.060 mmol) was charged to a vial in DCM (2 mL) whereupon 2,6-lutidine (0.1 mL) and TBSOTf (0.1 mL) were added. The reaction mixture was allowed to stir for one hour. Once reaction went to completion according to LCMS, it was quenched with saturated sodium bicarbonate solution and extracted with ethyl acetate. The organic layer was washed with 1M HCl solution to remove lutidine. The organic layer was dried in vacuo and moved to next step. Scheme 10, shown below, depicts such a process.

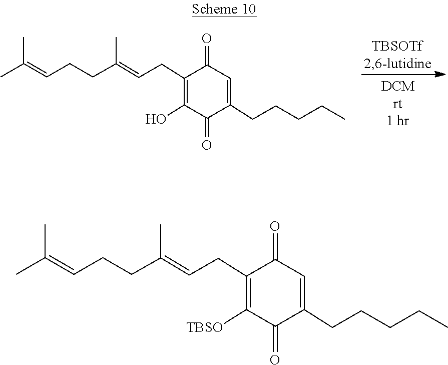

Starting material (20 mg, 0.060 mmol) was charged to a vial whereupon sodium dithionite in excess (52.2 mg, 0.3 mmol) in THF was added (2 mL). The reaction was allowed to stir for ten minutes, then 1M HCl solution was added. The reaction was allowed to stir ten minutes then added dimethylsulfate (30 mg, 0.24 mmol), and potassium carbonate (33 mg, 0.24 mmol) and allowed to stir overnight. Completion was checked via LCMS then filtered reaction through Celite. The mixture was washed with water (3×10 mL), then dried organic layer in vacuo. Reaction moved to next step without purification. Scheme 11, shown below, depicts such a process.

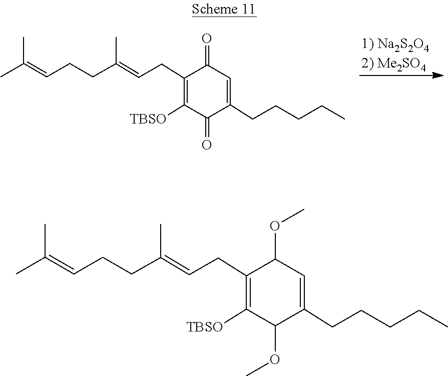

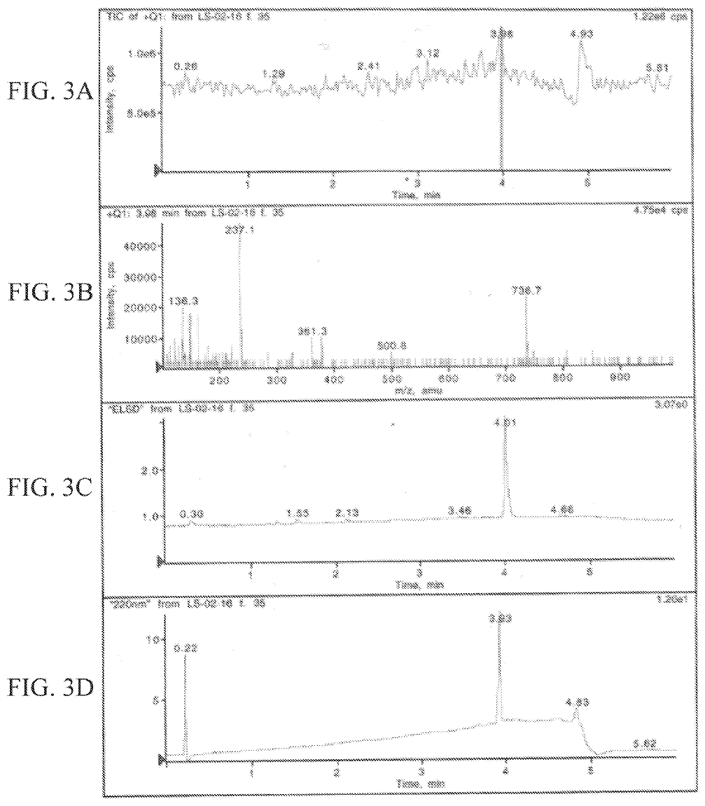

To a solution of the protected product (1.1 eq) in THF was added TBAF (1 eq). The solution was stirred at r.t for 1 h whereupon it was concentrated in vacuo and submitted for HPLC purification. The resulting product was 0.5 mg, 3% yield. [M+H]⁺ 361.3. FIGS. 3A-3D show the gas chromatography mass spectrometry (GC-MS) total ion chromatogram (TIC) spectra, the chromatography mass spectrometry (GC-MS) mass-to-charge ratio (m/z) spectra, the high-performance liquid chromatography evaporative light scattering detector (HPLC-ELSD) spectra, and the high-performance liquid chromatography ultraviolet (HPLC-UV) spectra at 220 nm for a compound of Formula XLII, respectively. Scheme 12, shown below, depicts such a process.

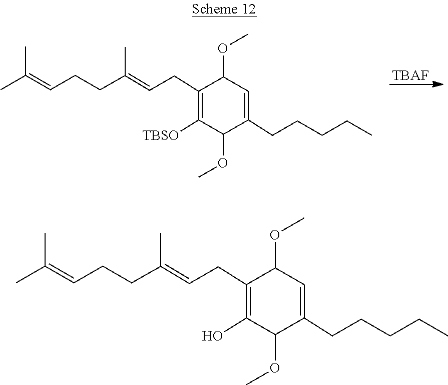

### Example 15: Synthesis of the Compound of Formula XLIII

A 4 mL vial with septa was charged with CBGQ (15 mg, 0.045 mmol), DCM (1 mL, 0.05M), followed by 2,6-lutidine (30 uL, 0.261 mmol, 5.5 eq) under argon. The reaction was cooled to 0° C. TBSOTf (14 uL in 100 ul of DCM prepared in a separate vial under argon) was added to the reaction dropwise at 0° C. The reaction was stirred at 0° C. for 30 min. The mixture was washed with NaHCO₃ (5 mL). The aqueous layer was extracted with EtOAc (3 mL), and the combined organic layers were dried over Na₂SO₄ and concentrated in vacuo to give a crude dark orange gum. Scheme 13, shown below, depicts such a process.

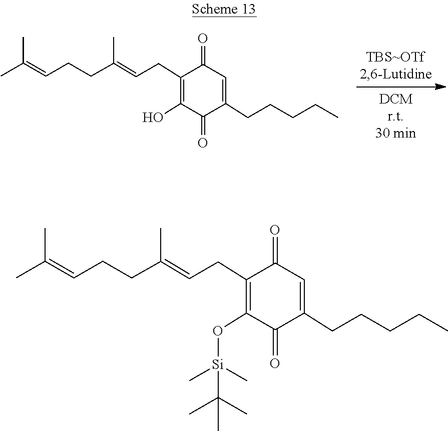

The TBS protected hydroxyquinone (16 mg) was dissolved in pyridine (1 ml) and acetic anhydride (0.5 ml) and left al room temp overnight. The solution was then concentrated in vacuo and dissolved in acetic anhydride (1 mL) and acetic acid (1 mL). Zinc (0.5 g) was added and the mixture was boiled under reflux for 30 min. The residue was filtered through a Celite plug, pyridine (2 ml) was used to wash the solid and the solution was left at room temp overnight after which it was poured into ice water and extracted with ethyl acetate (2×10 ml). The organics were concentrated and submitted for HPLC purification. Product containing fractions were concentrated together to give 6.8 mg, 0.0128 mmol and a 35% yield. Scheme 14, shown below, depicts such a process.

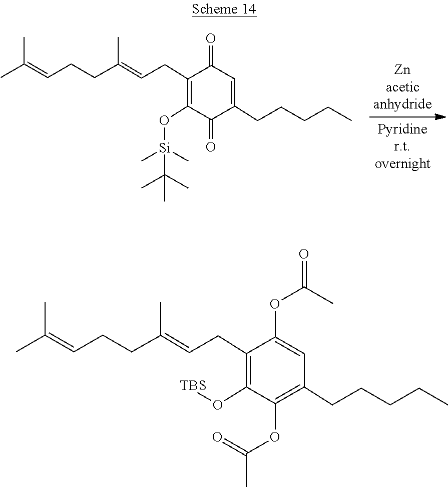

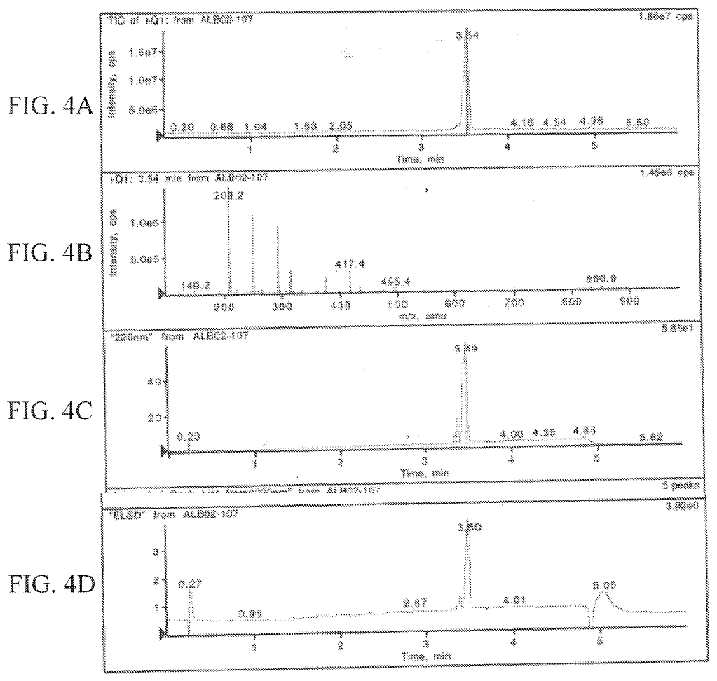

A solution of ALB02-106 TBS protected material (6.8 mg, 0.0128 mmol), in THF (1.28 mL, 0.01M) under argon was cooled to 0° C. and treated with TBAF (0.05M in THF, 0.28 mL, 0.0141 mmol, 1.1 equivalent). After 90 seconds the reaction was treated with sat. aqueous NaHCO₃ (1.5 mL). The mixture was extracted with Et₂O (3×1.5 mL). The combined organics were washed with brine (1 mL), dried over Na₂SO₄, filtered, concentrated in vacuo to give a crude residue. The crude material was further purified by flash chromatography to afford 3 mg, 56% yield. [M+H]⁺ 417.4. FIGS. 4A-4D show the gas chromatography mass spectrometry (GC-MS) total ion chromatogram (TIC) spectra, the chromatography mass spectrometry (GC-MS) mass-to-charge ratio (m/z) spectra, the high-performance liquid chromatography ultraviolet (HPLC-UV) spectra at 220 nm, and the high-performance liquid chromatography evaporative light scattering detector (HPLC-ELSD) spectra for a compound of Formula XLIII, respectively. Table 7 herein provides additional information of the peaks shown in FIG. 4C. Scheme 15, shown below, depicts such a process.

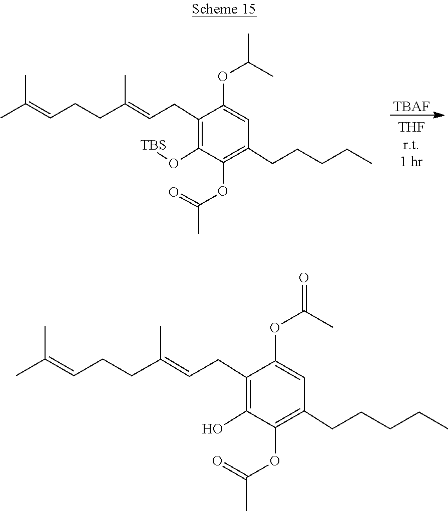

TABLE 7
Integrated Peak List of HPLC-UV Spectra

| Peak | RT | | | | | Start | End | Width | Baseline |
|---|---|---|---|---|---|---|---|---|---|
| # | (min) | Area | % Area | Height | % Height | (min) | (min) | (min) | Type |
| 3 | 3.47 | 1.145e2 | 50.776 | 5.245e1 | 41.119 | 3.41 | 3.48 | 0.07 | Valley |
| 4 | 3.49 | 8.587e1 | 38.087 | 5.584e1 | 43.780 | 3.48 | 3.53 | 0.06 | Expo. |
| | | | | | | | | | Skin |
| 2 | 3.38 | 1.853e1 | 8.218 | 1.406e1 | 11.025 | 3.36 | 3.41 | 0.05 | Valley |
| 1 | 3.36 | 5.997e0 | 2.660 | 4.955e0 | 3.885 | 3.31 | 3.36 | 0.05 | Valley |
| 5 | 3.57 | 5.838e−01 | 0.259 | 2.431e−01 | 0.191 | 3.53 | 3.59 | 0.06 | Expo. |
| | | | | | | | | | Dau |

Although the invention has been described with reference to embodiments and examples, it should be understood that numerous and various modifications can be made without departing from the spirit of the invention. Accordingly, the invention is limited only by the following claims.

1. A compound of formula (I) or formula (Ia): 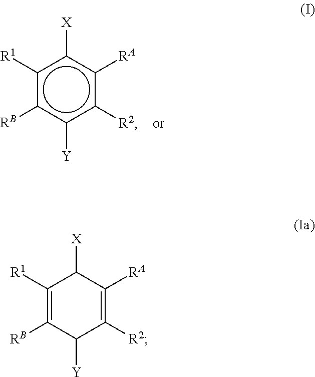
wherein each of R^{A} and R^{B} is independently selected from the group consisting of: —H, —F, —Cl, —Br, —I, —CH₃, —CH₂CH₃, —CF₃, —OH, —OCH₃, —OCH₂CH₃, —O(CO)NH₂, —O(CO)CH₃, —O(CO)CH₂CH₃, —CN, —NH₂, —NHCH₂CH₂CH₃, —NHCH₂CH₃, —N(CH₃)₂ and —NHCH(CH₃)₂;
wherein each of X and Y is independently selected from the group consisting of: —OH, ═O, —OCH₃, —O(CO)CH₃ and —O(CO)CH₂CH₃;
wherein R¹ is a moiety of formula (II): 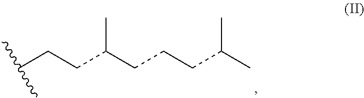
wherein each dashed bond is individually selected from representing a carbon-carbon single bond and representing a carbon-carbon double bond;
wherein R² is either a moiety of the formula (III-A), —(CH₂)ₙCH₃, wherein n in an integer greater than one and less than seven, or a moiety of the formula (III-B), —(CH₂)ₘ(CH═CH)(CH₂)ₚCH₃, wherein m is an integer greater than zero and less than four, and wherein p is an integer greater than or equal to zero and less than four, and wherein the sum of m and p is not greater than four; and
with the caveat that the compound is not any of the following compounds:

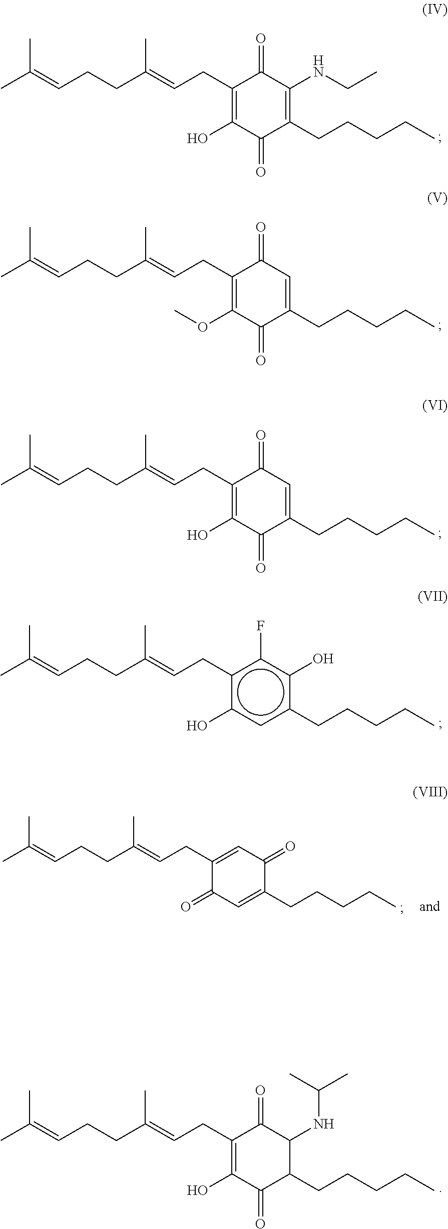

2. The compound of claim 1, wherein R^{B} is —OH.

3. The compound of claim 1, wherein R^{B} is selected from the group consisting of —OCH₃, —OCH₂CH₃, —O(CO)CH₃, —O(CO)CH₂CH₃, and —NH₂.

4. The compound of claim 1, wherein R^{A} is —H.

5. The compound of claim 1, wherein R^{A} is selected from the group consisting of —F, —Cl, and —Br.

6. The compound of claim 1, wherein each of X and Y is ═O.

7. The compound of claim 1, wherein each of X and Y is independently selected from the group consisting of —OCOCH₃ and —O(CO)CH₂CH₃.

8. The compound of claim 1, wherein Y is ═O.

9. The compound of claim 1, wherein R² is a five-carbon moiety.

10. The compound of claim 1, wherein Y is selected from the group consisting of —OCOCH₃ and —O(CO)CH₂CH₃.

11. The compound of claim 1, wherein the compound inhibits an activity selected from the group consisting of TNF, IL-6, IL-1β, IFNg, Lox5, Lox15, IL-10, CB2, and combinations thereof with an EC50 that is at most about 20 μM.

12. The compound of claim 1, wherein the compound has at least about 100-fold decreased toxicity to cells relative to staurosporine.

13. The compound of claim 1, wherein the compound has at least about 2 fold selectivity for inhibiting the CR2 receptor over the CB1 receptor.

14. A composition comprising the compound of claim 1 and at least one of a pharmaceutically effective carrier, a pharmaceutically effective diluent and a pharmaceutically effective excipient.

15. The composition claim 14, wherein the compound is formulated for intradermal, topical, transdermal, oral, buccal, sublingual, nasal or intravenous application.

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. A method of treating, ameliorating, or preventing the recurrence of an inflammatory condition selected from the group consisting of acute inflammation, chronic inflammation, developmental inflammation, meta-inflammation and inflammaging in a subject having an inflammatory condition, comprising:
administering an effective dose of a composition comprising (i) at least one of a pharmaceutically effective carrier, a pharmaceutically effective diluent, and a pharmaceutically effective excipient, and (ii) a compound of formula (I) or formula (Ia): 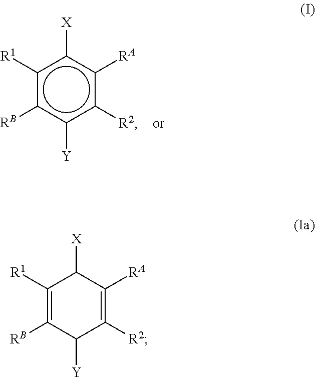
wherein R^{A} and R^{B} are each selected from the group consisting of:
—H, —F, —Cl, —Br, —I, —CH₃, —CH₂CH₃, —CF₃, —OH, —OCH₃, —OCH₂CH₃, —OCONH₂, —O(CO)CH₃, —O(CO)CH₂CH₃, —CN, —NH₂, —NHCH₂CH₂CH₃, —NHCH₂CH₃, —N(CH₃)₂ and —NHCH(CH₃)₂;
wherein each of X and Y are independently selected from the group consisting of:
—OH, ═O, —OCH₃, —OCOCH₃ and —OCOCH₂CH₃;
wherein R is a moiety of formula (II): 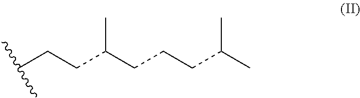
wherein each dashed line is independently selected from representing a carbon-carbon single bond and representing a carbon-carbon double bond;
wherein R² is either a moiety of the formula (III-A), —(CH₂)ₙCH₃, wherein n in an integer greater than one and less than seven, or a moiety of the formula (III-B), —(CH₂)ₘ(CH═CH)(CH₂)ₚCH₃, wherein m is an integer greater than zero and less than four, and wherein p is an integer greater than or equal to zero and less than four, and wherein the sum of m and p is not greater than four; and
with the caveat that the compound is not any of the following compounds:

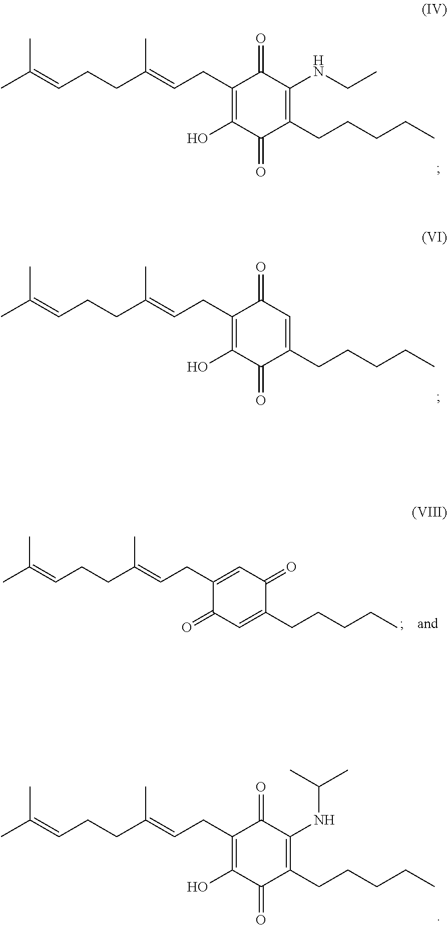

31. The method claim 30, wherein the chronic inflammation results from or is associated with at least one of anemia, arthritis, asthma, autoimmune disease, bone disease, bowel disease, cancer, cardiovascular disease, celiac disease, cerebrovascular disease, Crone's disease, diabetes, dysglycemia, eczema, fibromyalgia, gastrointestinal disorder, gingivitis, granulomatosis, Grave's disease, Hashimoto's disease, hemolytic anemia, inflammatory bowel disease, joint disease, leukemia, lupus, metabolic disease, muscular dystrophy, neuropathy, obesity, ocular disease, periodontitis, psoriasis, pulmonary disease, renal disease, rheumatoid arthritis, scleroderma, sclerosis, skin disease, thyroid disease, thyroiditis, ulceratic colitis, vitiligo, Wegener's disease, Castleman's disease, pulmonary arterial hypentension, atopic dermititus, and sciatica.

32. The method of claim 30, wherein the compound inhibits an activity selected from the group consisting of TNF, IL-6, IL-1β, IFNg, Lox5, Lox15, IL-10, CR2, and combinations thereof with an EC50 that is at most about 20 μM.

33. The method of claim 30, wherein the compound has at least about 100-fold decreased toxicity to cells relative to staurosporine.

34. The method of claim 30, wherein the compound has at least about 2 fold selectivity for inhibiting the CB2 receptor over the CB1 receptor.

35. (canceled)

36. A method of treating, ameliorating, reducing, or preventing the recurrence of a one or more symptom of a disease or disorder in a subject in need thereof, the method comprising:
administering a compound of formula (I) or formula (Ia): 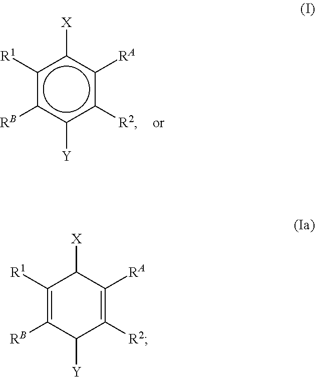
wherein each of R^{A} and R^{B} is independently selected from the group consisting of: —H, —F, —Cl, —Br, —I, —CH₃, —CH₂CH₃, —CF₃, —OH, —OCH₃, —OCH₂CH₃, —O(CO)NH₂, —O(CO)CH₃, —O(CO)CH₂CH₃, —CN, —NH₂, —NHCH₂CH₂CH₃, —NHCH₂CH₃, —N(CH₃)₂ and —NHCH(CH₃)₂;
wherein each of X and Y is independently selected from the group consisting of: —OH, ═O, —OCH₃, —O(CO)CH₃ and —O(CO)CH₂CH₃;
wherein R is a moiety of formula (II): 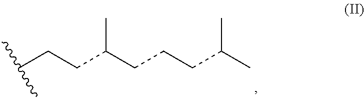
wherein each dashed bond is individually selected from representing a carbon-carbon single bond and representing a carbon-carbon double bond;
wherein R² is a moiety of formula (III): 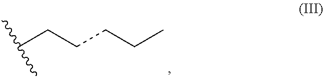
wherein the dashed bond is selected from representing a carbon-carbon single bond and representing a carbon-carbon double bond; and
wherein the disease or disorder is associated with the activity of or the expression of one or more of TNF, IL-6, IL-1β, IL10, IFNg, Lox5, CB2, and/or Lox15.

37. (canceled)

38. A compound selected from the group comprising:

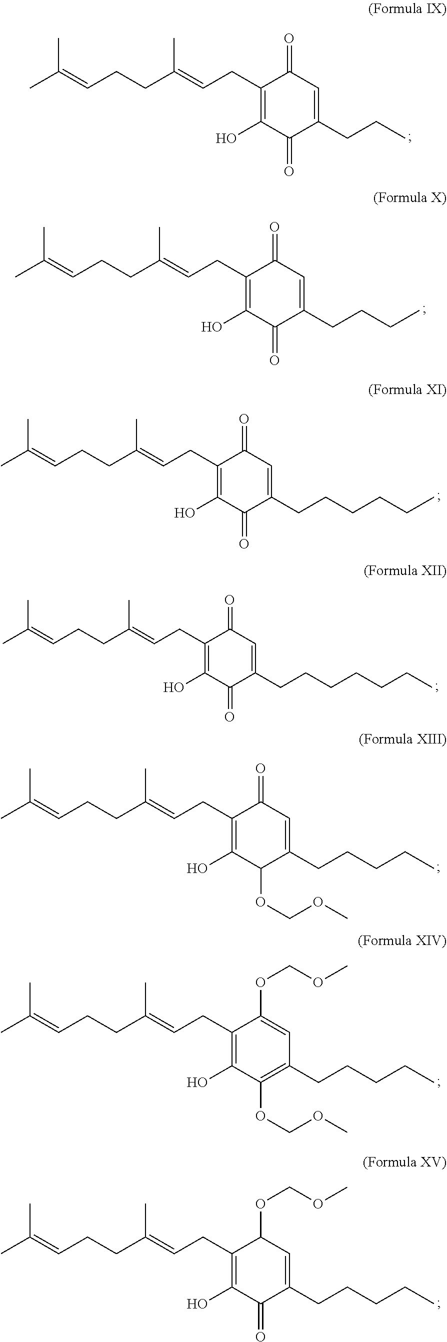

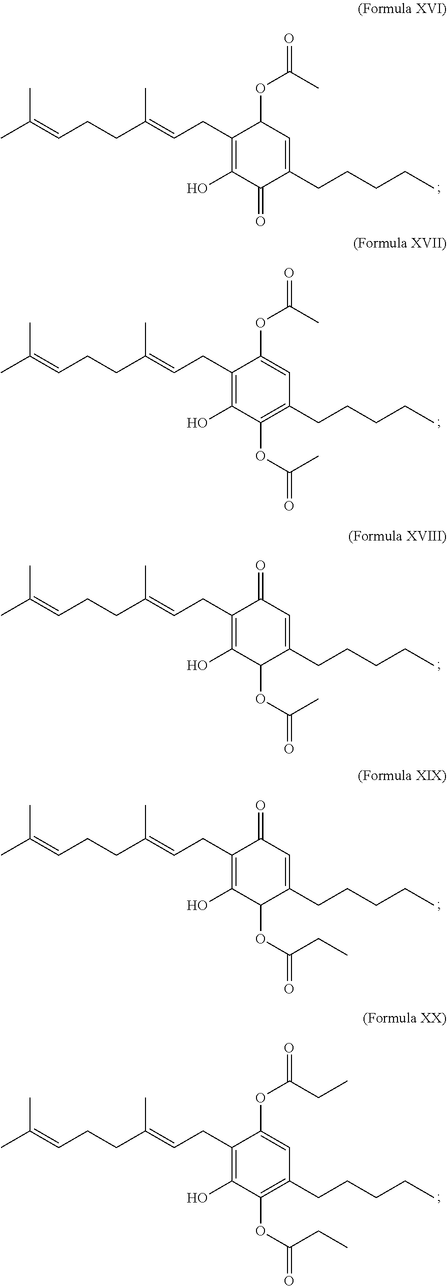

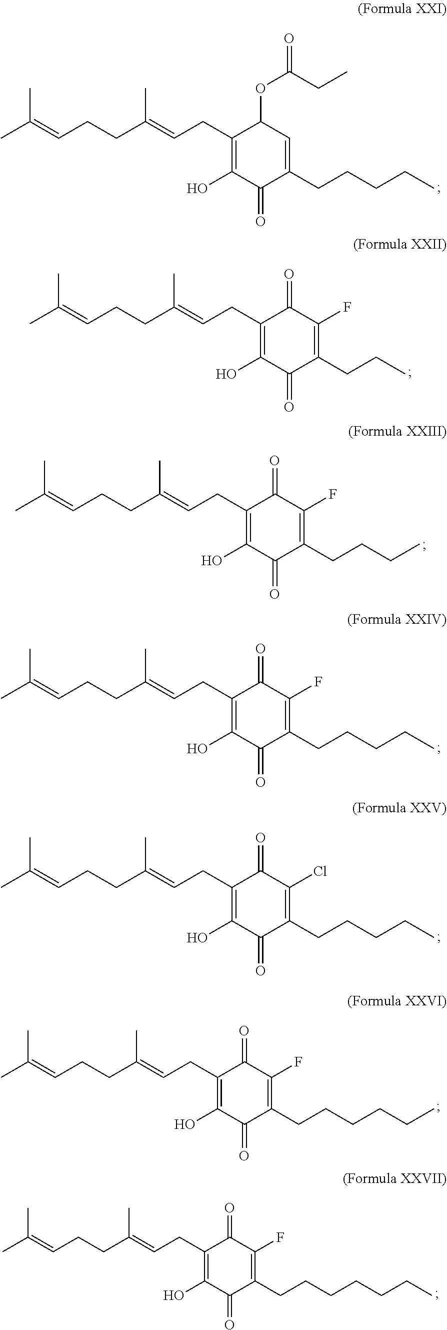

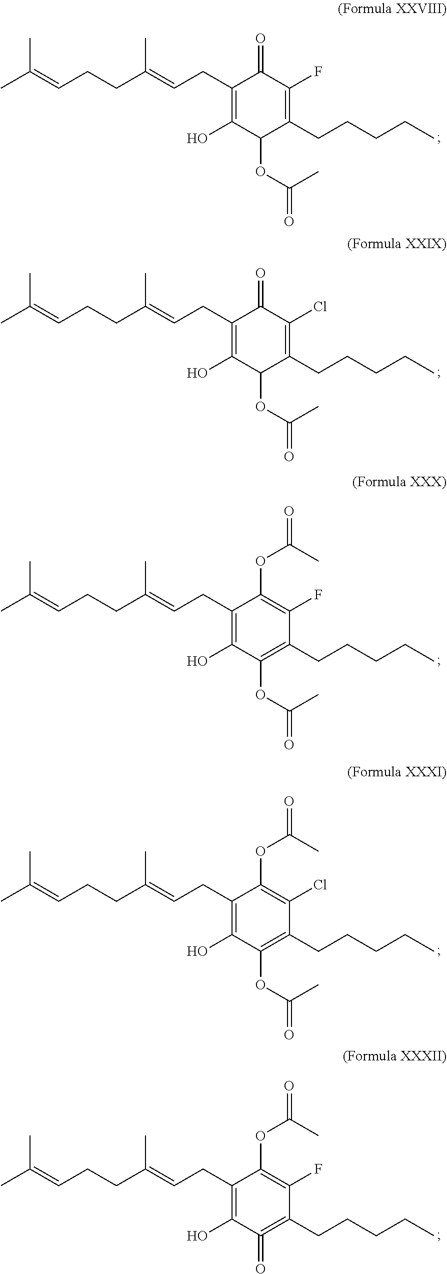

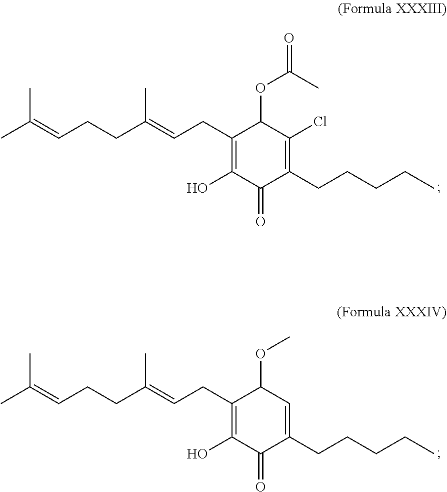

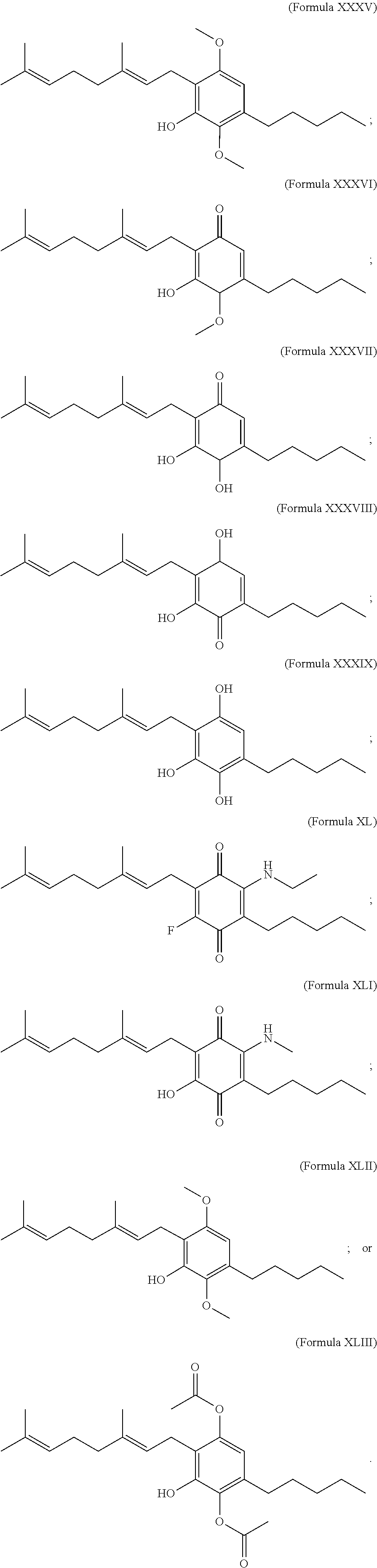

39. A composition comprising at least one compound of claim 38 and at least one of a pharmaceutically effective carrier, a pharmaceutically effective diluent and a pharmaceutically effective excipient.

40. (canceled)

41. (canceled)
